(19) United States
(12) Patent Application Publication
(10) Pub. No.: US 2018/0057462 A1
(43) Pub. Date: Mar. 1, 2018

(54) Substituted 2-difluoromethyl-nicotin(thio)carboxanilide derivatives and their use as fungicides

(71) Applicant: BAYER CROPSCIENCE AKTIENGESELLSCHAFT, MONHEIM AM RHEIN (DE)

(72) Inventors: CHRISTOPHE DUBOST, LA TOUR DE SALVAGNY (FR); SIMON MAECHLING, II, LYON (FR); PHILIPP WINTER, DUESSELDORF (DE); ULRIKE WACHENDORFF-NEUMANN, NEUWIED (DE); CYRIL MONTAGNE, MONHEIM AM RHEIN (DE); JEAN-PIERRE VORS, SAINTE FOY LES LYON (FR); STEPHANE BRUNET, ANDRE DE CORCY (FR)

(21) Appl. No.: 15/551,103

(22) PCT Filed: Feb. 15, 2016
(86) PCT No.: PCT/EP2016/053098

§ 371 Date: Aug. 15, 2017

(30) Foreign Application Priority Data
Feb. 18, 2015 (EP) 15155571.1

(51) Int. Cl. C07D 213/82; A01N 43/40

(52) U.S. Cl. CPC C07D 213/82; A01N 43/40

(57) ABSTRACT

The present invention relates to novel substituted 2-difluoromethyl-nicotin(thio)carboxanilides derivatives, to processes for preparing these compounds, to compositions comprising these compounds, and to the use thereof as biologically active compounds, especially for control of harmful microorganisms in crop protection and in the protection of materials.

The present invention relates to novel substituted 2-difluoromethyl-nicotin(thio)carboxanilides derivatives, to processes for preparing these compounds, to compositions comprising these compounds, and to the use thereof as biologically active compounds, especially for control of harmful microorganisms in crop protection and in the protection of materials and as plant growth regulators.

Since the ecological and economical demands made on modern crop protection agents are increasing constantly, for example with respect to activity spectrum, toxicity, selectivity, application rate, formation of residues and favourable manufacture, and there can furthermore be problems, for example, with resistances, there is a constant need to develop novel crop protection compositions, in particular fungicides, which, at least in some areas, have advantages over the known ones.

It is already known that numerous (hetero)cyclyl carboxanilides, among which Nicotin(thio)amide derivatives, have fungicidal properties (Cf. for example WO2005/074686, WO2006/131221, WO2005/075411, WO2005/095392, WO2005/063692, WO2006/02719, WO2007/017449, WO2007/128756 and WO2006/097490). In particular, 2-Chloro Nicotin(thio)amide derivatives of meta-substituted anilines are known to be active against, amongst others, Botrytis Cinerea and Pyricularia Oryzae, (DE2611601, J. Pesticide sci. 18, 1993, 49-57). The activity of these compounds is good. However, in some cases, for example at low application rates, it is sometimes unsatisfactory. Moreover there is no specific mentioning or exemplification made of any 2-difluoromethyl-nicotin(thio)amide derivatives of meta-substituted anilines.

It has now been found that, surprisingly, the present substituted 2-difluoromethyl-nicotin(thio) carboxanilide derivatives achieve at least some aspects of the objects mentioned and are suitable for use as crop protection compositions, especially as fungicides.

The present invention relates to compounds of the general formula (I)

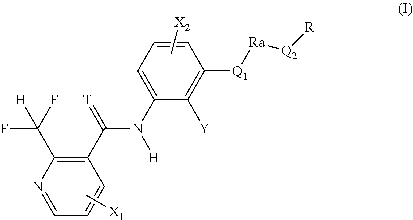

wherein
X₁ represents hydrogen, halogen, CN, NO₂, C₁-C₃-alkyl, C₁-C₃-haloalkyl, C₁-C₃-alkyloxy, C₁-C₃-haloalkyloxy, tri(C₁-C₃)alkylsilyl, C₁-C₃-alkylsulfanyl, C₁-C₃-haloalkylsulfanyl, C₁-C₃-alkylsulfonyl; C₁-C₃-haloalkylsulfonyl, C₁-C₃-alkylamino; di-C₁-C₃-alkylamino;
X₂ represents H, halogen, CN, NO₂, C₁-C₆-alkyl, C₁-C₆-haloalkyl, C₃-C₈-cycloalkyl, C₃-C₈-halocycloalkyl, C₁-C₆-alkyloxy, C₁-C₆-haloalkyloxy, tri(C₁-C₈)alkylsilyl, C₁-C₆-alkylsulfanyl, C₁-C₆-haloalkylsulfanyl, C₁-C₆-alkylsulfonyl; C₁-C₆-haloalkylsulfonyl, C₁-C₈-alkylamino; di-C₁-C₈-alkylamino;
Y represents hydrogen, halogen, C₁-C₃-alkyl, C₁-C₃-haloalkyl,
Q₁ represents O, S(O)ₘ, NR¹ or a bond;
m represents 0, 1 or 2;
R¹ represents hydrogen, C₁-C₆-alkyl, C₃-C₈-cycloalkyl, C₁-C₈-halogenoalkyl,
Ra represents a group

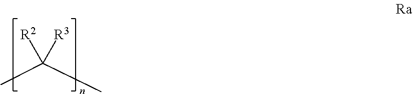

R² and R³ which can be the same or different independently of one another, represent hydrogen, halogen, CN, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy, C₁-C₈-alkylsulfanyl, C₃-C₈-cycloalkylsulfanyl, C₁-C₈-halogenoalkylsulfanyl, C₃-C₈-halogenocycloalkylsulfanyl;
R² and R³ also together with the carbon atom to which they are linked can form a C₃-C₇-cycloalkyl or a carbonyl;
n represents 0, 1, 2, 3, 4 or 5;
Q₂ represents O, S(O)ₘ, NR¹ or a bond,
R represents C₁-C₈-alkyl, C₃-C₈-cycloalkyl, C₁-C₈-halogenoalkyl, C₃-C₈-halogenocycloalkyl, C₂-C₈-alkenyl, C₃-C₈-cycloalkenyl, C₂-C₈-halogenoalkenyl, C₃-C₈-halogenocycloalkenyl, C₂-C₈-alkynyl, C₃-C₈-cycloalkynyl, C₂-C₈-halogenoalkynyl, C₃-C₈-halogenocycloalkynyl, a phenyl which can be substituted by up to 5 groups X₃, a saturated or unsaturated 5, 6 or 7 membered heterocycle which can be substituted by up to 5 groups X₃, a group ═N—O—R⁴, C₇-C₁₂-bicycloalkyl,
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl or tri(C₁-C₈)alkylsilyl-C₁-C₈-alkyl, or two vicinal substituents X₃ may be —OCH₂O—, —OCF₂O—, —O(CH₂)₂O—, —O(CF₂)₂O— or —N═CH—S—,
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃, C₁-C₈-alkyl phenyl which can be substituted by up to 5 groups X₃
T is O, S;
and salts, solvates, N-oxides, solvates of the salts and N-oxides thereof.

Preferred are 2-difluoromethyl-nicotin(thio) carboxanilide derivatives of the formula (I) where the radicals are described as follows:
X₁ represents hydrogen, halogen, CN, NO₂, C₁-C₃-alkyl, C₁-C₃-haloalkyl,
X₂ represents H, halogen, CN, NO₂, C₁-C₆-alkyl, C₁-C₆-haloalkyl, C₁-C₆-alkyloxy, C₁-C₆-haloalkyloxy,
Y represents hydrogen, halogen, methyl, ethyl, propyl, difluoromethyl, trifluoromethyl,
Q₁ represents O, S, NH or a bond,
Ra represents a group,

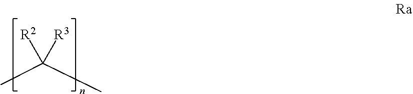

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy;
n represents 0, 1, 2, 3, 4 or 5;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃, a group ═N—O—R⁴;
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl;
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃, C₁-C₈-alkyl phenyl which can be substituted by up to 5 groups X₃;
T represents O;
Q₂ represents O, S or a bond.

More preferred are 2-difluoromethyl-nicotin(thio) carboxanilide derivatives of the formula (I) where the radicals are described as follows:
X₁ represents hydrogen, Chlorine, fluorine, bromine, methyl, trifluoromethyl;
X₂ represents hydrogen, Chlorine, fluorine, bromine, methyl, trifluoromethyl;
Y represents chlorine, methyl, trifluoromethyl,
Q₁ represents O, S, NH or a bond
Ra represents a group

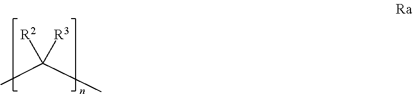

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃;
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl.
T represents O
Q₂ represents O, S or a bond.

Moreover preferred are 2-difluoromethyl-nicotin carboxanilide derivatives of the formula (II) where the radicals are described as follows:

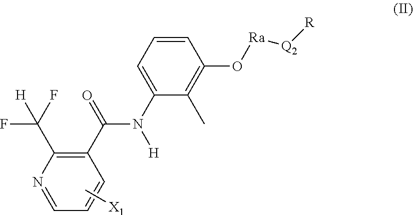

X₁ represents hydrogen, fluorine, chlorine, bromine, iodine, methyl, trifluoromethyl, difluoromethyl
Ra represents a group

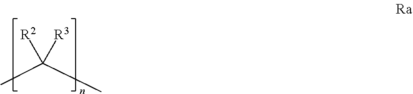

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃, a group ═N—O—R⁴,
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃;
Q₂ represents O or a bond.

Moreover preferred are 2-difluoromethyl-nicotin carboxanilide derivatives of the formula (II) where the radicals are described as follows:

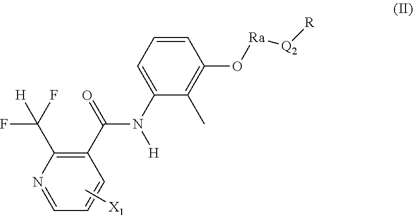

X₁ represents hydrogen, fluorine, chlorine, bromine, iodine, methyl, trifluoromethyl, difluoromethyl
Ra represents a group

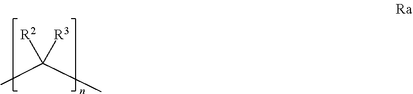

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃,
Q₂ represents O or a bond.

Moreover preferred are 2-difluoromethyl-nicotin carboxanilide derivatives of the formula (III) where the radicals are described as follows:

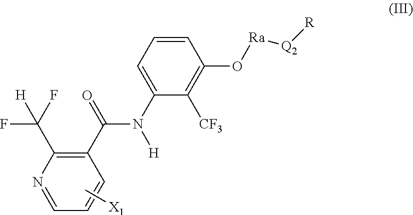

X₁ represents hydrogen, fluorine, chlorine, bromine, iodine, methyl, trifluoromethyl, difluoromethyl
Ra represents a group

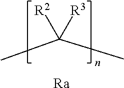

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃, a group ═N—O—R⁴;
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl;
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃;
Q₂ represents O or a bond.

Moreover preferred are 2-difluoromethyl-nicotin carboxanilide derivatives of the formula (III) where the radicals are described as follows:

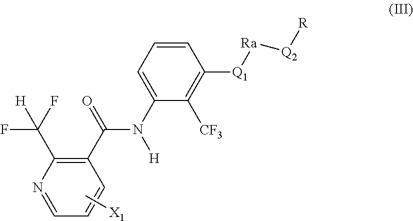

X₁ represents hydrogen, fluorine, chlorine, bromine, iodine, methyl, trifluoromethyl, difluoromethyl
Ra represents a group

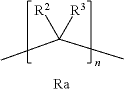

R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃;
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl;
Q₂ represents O or a bond.

In the definitions of the symbols given in the above formulae, collective terms were used, which are generally representative of the following substituents:

Halogen: fluorine, chlorine, bromine and iodine and preferably fluorine, chlorine, bromine and more preferably fluorine, chlorine.

Alkyl: saturated, straight-chain or branched hydrocarbyl radicals having 1 to 8, preferably 1 to 6 and more preferably 1 to 3 carbon atoms, for example (but not limited to) C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. This definition also applies to alkyl as part of a composite substituent, for example cycloalkylalkyl, hydroxyalkyl etc., unless defined elsewhere like, for example, alkylthio, alkylsufinyl, alkylsulphonyl, haloalkyl or haloalkylthio. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, formyl etc., are at the end.

Alkenyl: unsaturated, straight-chain or branched hydrocarbyl radicals having 2 to 8, preferably 2 to 6, carbon atoms and one double bond in any position, for example (but not limited to) C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl. This definition also applies to alkenyl as part of a composite substituent, for example haloalkenyl etc., unless defined elsewhere.

Alkynyl: straight-chain or branched hydrocarbyl groups having 2 to 8, preferably 2 to 6, carbon atoms and one triple bond in any position, for example (but not limited to) C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl. This definition also applies to alkynyl as part of a composite substituent, for example haloalkynyl etc., unless defined elsewhere.

Alkoxy: saturated, straight-chain or branched alkoxy radicals having 1 to 8, preferably 1 to 6 and more preferably 1 to 3 carbon atoms, for example (but not limited to) C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. This definition also applies to alkoxy as part of a composite substituent, for example haloalkoxy, alkynylalkoxy, etc., unless defined elsewhere.

Alkylthio: saturated, straight-chain or branched alkylthio radicals having 1 to 8, preferably 1 to 6 and more preferably 1 to 3 carbon atoms, for example (but not limited to) C₁-C₆-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio. This definition also applies to alkylthio as part of a composite substituent, for example haloalkylthio etc., unless defined elsewhere.

Alkoxycarbonyl: an alkoxy group which has 1 to 6, preferably 1 to 3, carbon atoms (as specified above) and is bonded to the skeleton via a carbonyl group (—CO—). This definition also applies to alkoxycarbonyl as part of a composite substituent, for example cycloalkylalkoxycarbonyl etc., unless defined elsewhere.

Alkylsulphinyl: saturated, straight-chain or branched alkylsulphinyl radicals having 1 to 8, preferably 1 to 6 and more preferably 1 to 3 carbon atoms, for example (but not limited to) C₁-C₆-alkylsulphinyl such as methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl. This definition also applies to alkylsulphinyl as part of a composite substituent, for example haloalkylsulphinyl etc., unless defined elsewhere.

Alkylsulphonyl: saturated, straight-chain or branched alkylsulphonyl radicals having 1 to 8, preferably 1 to 6 and more preferably 1 to 3 carbon atoms, for example (but not limited to) C₁-C₆-alkylsulphonyl such as methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl. This definition also applies to alkylsulphonyl as part of a composite substituent, for example alkylsulphonylalkyl etc., unless defined elsewhere.

Cycloalkyl: monocyclic, saturated hydrocarbyl groups having 3 to 10, preferably 3 to 8 and more preferably 3 to 6 carbon ring members, for example (but not limited to) cyclopropyl, cyclopentyl and cyclohexyl. This definition also applies to cycloalkyl as part of a composite substituent, for example cycloalkylalkyl etc., unless defined elsewhere.

Bicycloalkyl: fused rings; where the fusion occurs a) across a bond between two atoms (e.g. decalin), b) across a sequence of atoms (bridged, e.g. norbornane) or c) at a single atom (spirocyclic).

Cycloalkenyl: monocyclic, partially unsaturated hydrocarbyl groups having 3 to 10, preferably 3 to 8 and more preferably 3 to 6 carbon ring members, for example (but not limited to) cyclopropenyl, cyclopentenyl and cyclohexenyl. This definition also applies to cycloalkenyl as part of a composite substituent, for example cycloalkenylalkyl etc., unless defined elsewhere.

Cycloalkoxy: monocyclic, saturated cycloalkyloxy radicals having 3 to 10, preferably 3 to 8 and more preferably 3 to 6 carbon ring members, for example (but not limited to) cyclopropyloxy, cyclopentyloxy and cyclohexyloxy. This definition also applies to cycloalkoxy as part of a composite substituent, for example cycloalkoxyalkyl etc., unless defined elsewhere.

Haloalkyl: straight-chain or branched alkyl groups having 1 to 8, preferably 1 to 6 and more preferably 1 to 3 carbon atoms (as specified above), where some or all of the hydrogen atoms in these groups may be replaced by halogen atoms as specified above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl. This definition also applies to haloalkyl as part of a composite substituent, for example haloalkylaminoalkyl etc., unless defined elsewhere.

Haloalkenyl and haloalkynyl are defined analogously to haloalkyl except that, instead of alkyl groups, alkenyl and alkynyl groups are present as part of the substituent.

Haloalkoxy: straight-chain or branched alkoxy groups having 1 to 8, preferably 1 to 6 and more preferably 1 to 3 carbon atoms (as specified above), where some or all of the hydrogen atoms in these groups may be replaced by halogen atoms as specified above, for example (but not limited to) C₁-C₃-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as part of a composite substituent, for example haloalkoxyalkyl etc., unless defined elsewhere.

Haloalkylthio: straight-chain or branched alkylthio groups having 1 to 8, preferably 1 to 6 and more preferably 1 to 3 carbon atoms (as specified above), where some or all of the hydrogen atoms in these groups may be replaced by halogen atoms as specified above, for example (but not limited to) C₁-C₃-haloalkylthio such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as part of a composite substituent, for example haloalkylthioalkyl etc., unless defined elsewhere.

Heteroaryl: 5 or 6-membered, fully unsaturated monocyclic ring system containing one to four heteroatoms from the group of oxygen, nitrogen and sulphur; if the ring contains more than one oxygen atom, they are not directly adjacent;

5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazol-1-yl, 1-imidazolyl, 1,2,3-triazol-1-yl and 1,3,4-triazol-1-yl;

6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl;

benzofused 5-membered heteroaryl containing one to three nitrogen atoms or one nitrogen atom and one oxygen or sulphur atom: for example (but not limited to) indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl;

benzofused 6-membered heteroaryl which contains one to three nitrogen atoms: for example (but not limited to) quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

This definition also applies to heteroaryl as part of a composite substituent, for example heteroarylalkyl etc., unless defined elsewhere.

Heterocyclyl: three- to fifteen-membered, preferably three- to nine-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl. This definition also applies to heterocyclyl as part of a composite substituent, for example heterocyclylalkyl etc., unless defined elsewhere.

Leaving group: S_{N}1 or S_{N}2 leaving group, for example chlorine, bromine, iodine, alkylsulphonates (—OSO₂-alkyl, e.g. —OSO₂CH₃, —OSO₂CF₃) or arylsulphonates (—OSO₂-aryl, e.g. —OSO₂Ph, —OSO₂PhMe).

Not included are combinations which are against natural laws and which the person skilled in the art would therefore exclude based on his/her expert knowledge. Ring structures having three or more adjacent oxygen atoms, for example, are excluded.

Illustration of the Processes and Intermediates

Carboxamides of the formula (I-a), i.e. carboxamides of formula (I) wherein T represents oxygen, are obtained when carbonyl halides or acids of formula (VII) are reacted with amines of formula (VIII) if appropriate in the presence of a coupling agent, if appropriate in the presence of an acid binder and if appropriate in the presence of a diluent [Process (a)]:

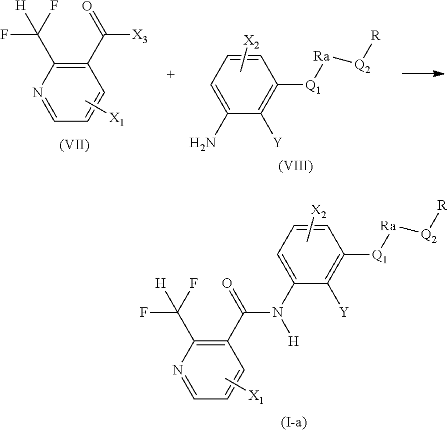

The formula (VII) provides a general definition of the carbonyl halides or acids required as starting materials for carrying out the Process (a) according to the invention.

In this formula (VII) X₁ has generally and preferably those meanings which have already been mentioned for this radical in connection with the description of the compounds of the formula (I). X³ represents halogen, hydroxyl, an activated hydroxyl group or a C₁-C₆ alkoxy group, preferably represents fluorine, chlorine, hydroxyl or a C₁-C₆ alkoxy group particularly preferably chlorine, C₁-C₆ alkoxy group or hydroxyl.

An activated hydroxyl group shall mean that the hydroxyl forms together with the adjacent carbonyl an ester which spontaneously reacts with an amino group. Common activated esters include p-nitrophenyl, pentafluorophenyl, succinimido esters or phosphorous anhydrides.

The carbonyl halides or acids of the formula (VII) can be prepared using similar procedures to the ones described in Chem. Commun., 2008, 4207-4209.

The formula (VIII) provides a general definition of the amines required as starting materials for carrying out the Process (a) according to the invention.

In this formula (VIII) Y, Q₁, Ra, Q₂, R and X₂ have generally, preferably, particularly preferably, very particularly preferably those meanings which have already been mentioned for these radicals in connection with the description of the compounds of the formula (I).

Thiocarboxamides of the formula (I-b), i.e. carboxamides of formula (I) wherein T represents sulfur, are obtained when carboxamides of the formula (I-a) are reacted with a thionating agent, if appropriate in the presence of a diluent, and if appropriate in the presence of a catalytic or stoichiometric or more quantity of a base [Process (b)]:

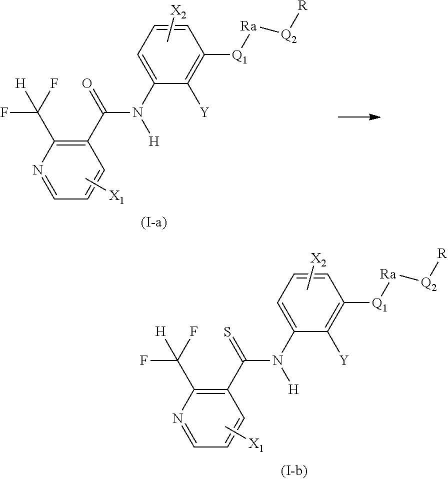

Compounds of formula (VIII) used as starting materials in process (a) are prepared by reacting phenols of formula (XI) if appropriate in the presence of a coupling agent, if appropriate in the presence of an acid binder and if appropriate in the presence of a diluent [Process (c)]:

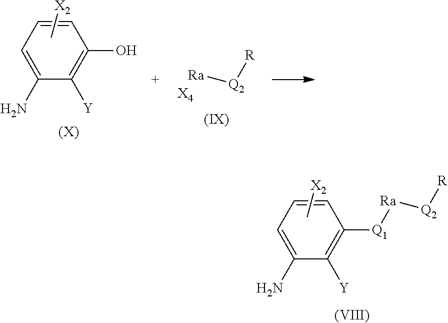

The formula (X) provides a general definition of the compounds required as starting materials for carrying out the process (c) according to the invention.

In this formula, (X), Y and X₂ have generally and preferably those meanings which have already been mentioned for these radicals in connection with the description of the compounds of the formula (I).

Compounds of formula (X) used as starting materials are commercially available

The formula (IX) provides a general definition of the compounds required as starting materials for carrying out the process (c) and process (d) according to the invention.

In this formula (IX), Ra, Q₂ and R have generally, preferably, particularly preferably, very particularly preferably those meanings which have already been mentioned for these radicals in connection with the description of the compounds of the formula (I). X₄ represents halogen or hydroxyl or an activated hydroxyl group, preferably represents bromine or hydroxyl. Common activated hydroxyl group include mesylate, p-toluene sulfonate or triflate groups.

The compounds of the formula (IX) are commercially available or can be prepared from commercially available starting material using known procedures (cf. R. C. Larock Comprehensive organic transformations, 1989, VCH publishers) Carboxamides of the formula (I-e), i.e. carboxamides of formula (I) wherein T represents oxygen, Q₁ and Q₂ represent a bond and n represents 0 are also obtained when bromides of formula (I-d) are reacted with compounds of formula (XI) if appropriate in the presence of a catalyst, if appropriate in the presence of an acid binder and if appropriate in the presence of a diluent [Process (d)]:

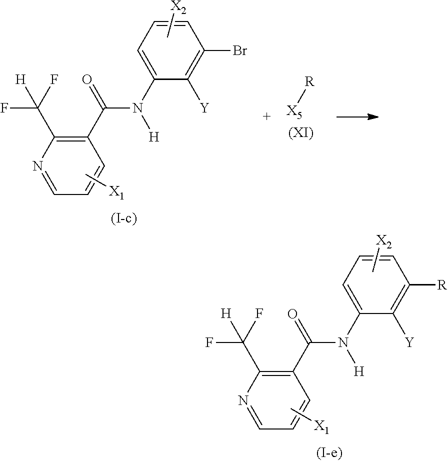

The formula (I-c) provides a general definition of the bromides required as starting materials for carrying out the Process (d) according to the invention.

In this formula (I-c), X₁, X₂, and Y have generally and preferably those meanings which have already been mentioned for these radicals in connection with the description of the compounds of the formula (I).

The formula (XI) provides a general definition of the compounds required as starting materials for carrying out the Process (e) according to the invention.

In this formula (XI), R have generally, preferably, particularly preferably, very particularly preferably those meanings which have already been mentioned for this radical in connection with the description of the compounds of the formula (I). X₅ represents a metallic residue such as, for example, a boronic acid, a trifluoroborate salk, a boronic ester, a zinc halide, a magnesium halide, a trialkylstannane.

The compounds of the formula (XI) are commercially available or can be prepared from commercially available starting material using known procedures (cf. R. C. Larock Comprehensive organic transformations, 1989, VCH publishers)

Carboxamides of the formula (I-c) can be obtained using process (a).

Suitable diluents for carrying out the processes (a), (b), (c) and (d) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decaline; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; their mixtures with water or pure water.

When carrying out the processes (a), (b) (c) and (d) according to the invention, the reaction temperatures can be varied within a relatively wide range. In general, the process is carried out at temperatures of from 0° C. to 150° C., preferably at temperatures of from 20° C. to 110° C.

The Processes (a), (b), (c), (d) are generally carried out under atmospheric pressure. However, it is also possible to operate under elevated or reduced pressure—in general between 0.1 bar and 100 bar.

The Process (a) according to the invention is, if appropriate, carried out in the presence of a suitable acid acceptor when X³ represents halogen. Suitable acid acceptors are all customary inorganic or organic bases. These preferably include alkaline earth metal or alkali metal hydrides, hydroxides, amides, alkoholates, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The Process (a) according to the invention is, if appropriate, carried out in the presence of a suitable coupling agent when X³ represents hydroxyl. Suitable coupling agents are all customary carbonyl activators. These preferably include N-[3-(dimethylamino)propyl]-N′-ethyl-carbodiimide-hydrochloride, N,N′-di-sec-butylcarbodiimide, N,N′-dicyclohexylcarbodiimide, N,N′-diisopropylcarbodiimide, 1-(3-(dimethylamino)propyl)-3-ethylcarbodiimide methiodide, 2-bromo-3-ethyl-4-methylthiazolium tetrafluoroborate, N,N-bis[2-oxo-3-oxazolidinyl]phosphorodiamidic chloride, chlorotripyrrolidinophosphonium hexafluorophosphate, bromtripyrrolidinophosphonium hexafluorophosphate, O-(1H-benzotriazol-1-yloxy)tris(dimethylamino)phosphonium hexafluorophosphate, O-(1H-benzotriazol-1-yl)-N,N,N′,N′-tetramethyluronium hexafluorophosphate, O-(1H-benzotriazol-1-yl)-N,N,N′,N′-bis(tetramethylene)uronium hexafluorophosphate, O-(1H-benzotriazol-1-yl)-N,N,N′,N′-bis(tetra-methylene)uronium tetrafluoroborate, N,N,N′,N′-bis(tetramethylene)chlorouronium tetrafluoroborate, O-(7-azabenzotriazol-1-yl)-N,N,N,N-tetramethyluronium hexafluorophosphate, 1-hydroxybenzotriazoles and Propylphosphonic anhydride. These reagents can be employed separately, but also in combination.

Suitable thionating agents for carrying out process (b) according to the invention can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in J. Chem. Soc., Perkin 1 2001, 358.

Suitable bases for carrying out the process (b) according to the invention can be inorganic and organic bases which are customary for such reactions. Preference is given to using alkaline earth metal or alkali metal hydroxides, such as sodium hydroxide, calcium hydroxide, potassium hydroxide or other ammonium hydroxide derivatives; alkali metal carbonates, such as sodium carbonate, potassium carbonate, potassium bicarbonate, sodium bicarbonate; alkali metal or alkaline earth metal acetates, such as sodium acetate, potassium acetate, calcium acetate; and also tertiary amines, such as trimethylamine, triethylamine, tributylamine, N,N-dimethylaniline, pyridine, N methylpiperidine, N,N-dimethyl [symbol] aminopyridine, diazabicyclooctane (DABCO), diazabicyclononene (DBN) or diaza-bicycloundecene (DBU).

Suitable acid binders for carrying out the process (c) according to the invention can be inorganic and organic bases which are customary for such reactions. Preference is given to using alkaline earth metal or alkali metal hydroxides, such as sodium hydroxide, calcium hydroxide, potassium hydroxide or other ammonium hydroxide derivatives; alkali metal carbonates, such as sodium carbonate, potassium carbonate, potassium bicarbonate, sodium bicarbonate; alkali metal or alkaline earth metal acetates, such as sodium acetate, potassium acetate, calcium acetate; and also tertiary amines, such as trimethylamine, triethylamine, tributylamine, N,N-dimethylaniline, pyridine, N methylpiperidine, N,N-dimethyl [symbol] aminopyridine, diazabicyclooctane (DABCO), diazabicyclononene (DBN) or diaza-bicycloundecene (DBU).

Suitable activators for carrying out the process (c) according to the invention can be a combination of a dialkylazodicarboxylate ester derivative and a trialkyl or triarylphosphine. Dialkylazodicarboxylate ester derivatives can be, for example, diethylazodicarboxylate, diisopropylazodicarboxylate, Di-2-methoxyethyl azodicarboxylate, or Di-p-chlorobenzyl Azodicarboxylate. Triaklyl or triarylphosphine can be, for example, triphenyl phosphine or tributyl phosphine.

Suitable catalyst for carrying out process (d) according to the invention may be chosen from metal salt or complex. Suitable metal derivatives for this purpose are based on palladium or copper. Suitable metal salts or complexes for this purpose are palladium chloride, palladium acetate, tetrakis(triphenylphosphine)palladium, bis(triphenylphosphine)palladium dichloride or 1,1′-bis(diphenylphosphino) ferrocenepalladium(II) chloride, copper iodide, copper bromide, copper thiophene carboxylate, copper trifluoromethane sulfonate, copper (I) oxide.

It is also possible to generate a palladium complex in the reaction mixture by separate addition to the reaction of a palladium salt and a ligand or salt, such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine or 1,3-bis(2,4,6-trimethyl-phenyl)imidazolium chloride.

It is also advantageous to choose the appropriate catalyst and/or ligand from commercial catalogues such as “Metal Catalysts for Organic Synthesis” by Strem Chemicals or “Phosphorous Ligands and Compounds” by Strem Chemicals.

Suitable acid binders for carrying out the process (d) according to the invention can be inorganic and organic bases which are customary for such reactions. Preference is given to using alkaline earth metal or alkali metal hydroxides, such as sodium hydroxide, calcium hydroxide, potassium hydroxide or other ammonium hydroxide derivatives; alkali metal carbonates, such as sodium carbonate, potassium carbonate, potassium bicarbonate, sodium bicarbonate; alkali metal or alkaline earth metal acetates, such as sodium acetate, potassium acetate, calcium acetate; and also tertiary amines, such as trimethylamine, triethylamine, tributylamine, N,N-dimethylaniline, pyridine, N methylpiperidine, N,N-dimethyl [symbol] aminopyridine, diazabicyclooctane (DABCO), diazabicyclononene (DBN) or diaza-bicycloundecene (DBU).

Isomers

Depending on the nature of the substituents, the compounds of the formula (I) to (V) may be in the form of geometric and/or optically active isomers or corresponding isomer mixtures in different compositions. These stereoisomers are, for example, enantiomers, diastereomers, atropisomers or geometric isomers. Accordingly, the invention encompasses both pure stereoisomers and any mixture of these isomers.

Methods and Uses

The invention also relates to a method for controlling unwanted microorganisms, characterized in that the compounds of the formula (I) to (V) are applied to the microorganisms and/or in their habitat.

The invention further relates to seed which has been treated with at least one compound of the formula (I) to (V).

The invention finally provides a method for protecting seed against unwanted microorganisms by using seed treated with at least one compound of the formula (I) to (V).

The compounds of the formula (I) to (V) have potent microbicidal activity and can be used for control of unwanted microorganisms, such as fungi and bacteria, in crop protection and in the protection of materials.

The compounds of the formula (I) to (V) have very good fungicidal properties and can be used in crop protection, for example for control of Plasmodiophoromycetes, Oomycetes, Chytridiomycetes, Zygomycetes, Ascomycetes, Basidiomycetes and Deuteromycetes.

Bactericides can be used in crop protection, for example, for control of Pseudomonadaceae, Rhizobiaceae, Enterobacteriaceae, Corynebacteriaceae and Streptomycetaceae.

The compounds of the formula (I) to (V) can be used for curative or protective control of phytopathogenic fungi. The invention therefore also relates to curative and protective methods for controlling phytopathogenic fungi by the use of the inventive active ingredients or compositions, which are applied to the seed, the plant or plant parts, the fruit or the soil in which the plants grow.

Plants

All plants and plant parts can be treated in accordance with the invention. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips and seeds.

Plants which can be treated in accordance with the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

Pathogens

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;

diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

fruit rot caused, for example, by Aspergillus species, for example Aspergillus flavus; Botrytis species, for example Botrytis cinerea; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum;

seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;

cancers, galls and witches' broom caused, for example, by Nectria species, for example Nectria galligena;

wilt diseases caused, for example, by Monilinia species, for example Monilinia laxa;

deformations of leaves, flowers and fruits caused, for example, by Exobasidium species, for example Exobasidium vexans; Taphrina species, for example Taphrina deformans;

degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

Preference is given to controlling the following diseases of soya beans:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

and improved photosynthetic efficiency.

Mycotoxins

In addition, compounds of the formula (I) to (V) can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

Material Protection

The compounds of the formula (I) to (V) can also be used in the protection of materials, for protection of industrial materials against attack and destruction by phytopathogenic fungi.

In addition, the compounds of the formula (I) to (V) can be used as antifouling compositions, alone or in combinations with other active ingredients.

Industrial materials in the present context are understood to mean inanimate materials which have been prepared for use in industry. For example, industrial materials which are to be protected by inventive compositions from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compounds of the formula (I) to (V) may prevent adverse effects, such as rotting, decay, discoloration, decoloration or formation of mould.

In the case of treatment of wood the compounds of the formula (I) to (V) may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting a composition according to the invention; this includes for example direct application, spraying, dipping, injection or any other suitable means.

In addition, the compounds of the formula (I) to (V) can be used to protect objects which come into contact with saltwater or brackish water, especially hulls, screens, nets, buildings, moorings and signalling systems, from fouling.

The compounds of the formula (I) to (V) can also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The inventive compositions may prevent adverse effects, such as rotting, decay, discoloration, decoloration or formation of mould.

Microorganisms capable of degrading or altering the industrial materials include, for example, bacteria, fungi, yeasts, algae and slime organisms. The compounds of the formula (I) to (V) preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Formulations

The present invention further relates to a composition for controlling unwanted microorganisms, comprising at least one of compounds of the formula (I) to (V). These are preferably fungicidal compositions which comprise agriculturally suitable auxiliaries, solvents, carriers, surfactants or extenders.

According to the invention, a carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid carriers include: for example ammonium salts and natural rock flours, such as kaolins, clays, talc, chalk, quartz, attapulgite, montmorillonite or diatomaceous earth, and synthetic rock flours, such as finely divided silica, alumina and silicates; useful solid carriers for granules include: for example, crushed and fractionated natural rocks such as calcite, marble, pumice, sepiolite and dolomite, and also synthetic granules of inorganic and organic flours, and granules of organic material such as paper, sawdust, coconut shells, maize cobs and tobacco stalks; useful emulsifiers and/or foam-formers include: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Additionally suitable are oligo- or polymers, for example those derived from vinylic monomers, from acrylic acid, from EO and/or PO alone or in combination with, for example, (poly)alcohols or (poly)amines. It is also possible to use lignin and its sulphonic acid derivatives, unmodified and modified celluloses, aromatic and/or aliphatic sulphonic acids and also their adducts with formaldehyde.

The active ingredients can be converted to the customary formulations, such as solutions, emulsions, wettable powders, water- and oil-based suspensions, powders, dusts, pastes, soluble powders, soluble granules, granules for broadcasting, suspoemulsion concentrates, natural products impregnated with active ingredient, synthetic substances impregnated with active ingredient, fertilizers and also microencapsulations in polymeric substances.

The active ingredients can be applied as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural products impregnated with active ingredient, synthetic substances impregnated with active ingredient, fertilizers and also microencapsulations in polymeric substances. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

The formulations mentioned can be prepared in a manner known per se, for example by mixing the active ingredients with at least one customary extender, solvent or diluent, emulsifier, dispersant and/or binder or fixing agent, wetting agent, a water repellent, if appropriate siccatives and UV stabilizers and if appropriate dyes and pigments, antifoams, preservatives, secondary thickeners, stickers, gibberellins and also other processing auxiliaries.

The present invention includes not only formulations which are already ready for use and can be deployed with a suitable apparatus to the plant or the seed, but also commercial concentrates which have to be diluted with water prior to use.

The compounds of the formula (I) to (V) may be present as such or in their (commercial) formulations and in the use forms prepared from these formulations as a mixture with other (known) active ingredients, such as insecticides, attractants, sterilants, bactericides, acaricides, nematicides, fungicides, growth regulators, herbicides, fertilizers, safeners and/or semiochemicals.

The auxiliaries used may be those substances which are suitable for imparting particular properties to the composition itself or and/or to preparations derived therefrom (for example spray liquors, seed dressings), such as certain technical properties and/or also particular biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are understood to mean liquids which are gaseous at standard temperature and under standard pressure, for example aerosol propellants such as halohydrocarbons, or else butane, propane, nitrogen and carbon dioxide.

In the formulations it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids. Further additives may be mineral and vegetable oils.

If the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Compositions comprising compounds of the formula (I) to (V) may additionally comprise further components, for example surfactants. Suitable surfactants are emulsifiers and/or foam formers, dispersants or wetting agents having ionic or nonionic properties, or mixtures of these surfactants. Examples thereof are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Further additives may be perfumes, mineral or vegetable, optionally modified oils, waxes and nutrients (including trace nutrients), such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability.

If appropriate, other additional components may also be present, for example protective colloids, binders, adhesives, thickeners, thixotropic substances, penetrants, stabilizers, sequestering agents, complex formers. In general, the active ingredients can be combined with any solid or liquid additive commonly used for formulation purposes.

The formulations contain generally between 0.05 and 99% by weight, 0.01 and 98% by weight, preferably between 0.1 and 95% by weight, more preferably between 0.5 and 90% of active ingredient, most preferably between 10 and 70 percent by weight.

The formulations described above can be used for controlling unwanted microorganisms, in which the compositions comprising compounds of the formula (I) to (V) are applied to the microorganisms and/or in their habitat.

Mixtures

Compounds of the formula (I) to (V) can be used as such or in formulations thereof and can be mixed with known fungicides, bactericides, acaricides, nematicides or insecticides, in order thus to broaden, for example, the activity spectrum or to prevent development of resistance.

Useful mixing partners include, for example, known fungicides, insecticides, acaricides, nematicides or else bactericides (see also Pesticide Manual, 14th ed.).

A mixture with other known active ingredients, such as herbicides, or with fertilizers and growth regulators, safeners and/or semiochemicals, is also possible.

Seed Treatment

The invention furthermore includes a method for treating seed.

A further aspect of the present invention relates in particular to seeds (dormant, primed, pregerminated or even with emerged roots and leaves) treated with at least one of the compounds of the formula (I) to (V). The inventive seeds are used in methods for protection of seeds and emerged plants from the seeds from phytopathogenic harmful fungi. In these methods, seed treated with at least one inventive active ingredient is used.

The compounds of the formula (I) to (V) are also suitable for the treatment of seeds and young seedlings. A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

It is also desirable to optimize the amount of the active ingredient used so as to provide the best possible protection for the seeds, the germinating plants and emerged seedlings from attack by phytopathogenic fungi, but without damaging the plants themselves by the active ingredient used. In particular, methods for the treatment of seed should also take into consideration the intrinsic phenotypes of transgenic plants in order to achieve optimum protection of the seed and the germinating plant with a minimum of crop protection compositions being employed.

The present invention therefore also relates to a method for protecting seeds, germinating plants and emerged seedlings against attack by animal pests and/or phytopathogenic harmful microorganisms by treating the seeds with an inventive composition. The invention also relates to the use of the compositions according to the invention for treating seeds for protecting the seeds, the germinating plants and emerged seedlings against animal pests and/or phytopathogenic microorganisms. The invention further relates to seeds which has been treated with an inventive composition for protection from animal pests and/or phytopathogenic microorganisms.

One of the advantages of the present invention is that the treatment of the seeds with these compositions not only protects the seed itself, but also the resulting plants after emergence, from animal pests and/or phytopathogenic harmful microorganisms. In this way, the immediate treatment of the crop at the time of sowing or shortly thereafter protect plants as well as seed treatment in prior to sowing. It is likewise considered to be advantageous that the inventive active ingredients or compositions can be used especially also for transgenic seed, in which case the plant which grows from this seed is capable of expressing a protein which acts against pests, herbicidal damage or abiotic stress. The treatment of such seeds with the inventive active ingredients or compositions, for example an insecticidal protein, can result in control of certain pests. Surprisingly, a further synergistic effect can be observed in this case, which additionally increases the effectiveness for protection against attack by pests, microorganisms, weeds or abiotic stress.

The compounds of the formula (I) to (V) are suitable for protection of seed of any plant variety which is used in agriculture, in the greenhouse, in forests or in horticulture. More particularly, the seed is that of cereals (such as wheat, barley, rye, millet and oats), oilseed rape, maize, cotton, soybeen, rice, potatoes, sunflower, beans, coffee, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

As also described below, the treatment of transgenic seed with the inventive active ingredients or compositions is of particular significance. This refers to the seed of plants containing at least one heterologous gene which allows the expression of a polypeptide or protein, e.g. having insecticidal properties. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originates from Bacillus sp., in which case the gene product is effective against the European corn borer and/or the Western corn rootworm. Particularly preferably, the heterologous genes originate from Bacillus thuringiensis.

In the context of the present invention, the inventive composition is applied to seeds either alone or in a suitable formulation. Preferably, the seed is treated in a state in which it is sufficiently stable for no damage to occur in the course of treatment. In general, seeds can be treated at any time between harvest and some time after sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again, or seeds just after priming, or seeds stored in primed conditions or pregerminated seeds, or seeds sown on nursery trays, tapes or paper.

When treating the seeds, it generally has to be ensured that the amount of the inventive composition applied to the seed and/or the amount of further additives is selected such that the germination of the seed is not impaired, or that the resulting plant is not damaged. This must be ensured particularly in the case of active ingredients which can exhibit phytotoxic effects at certain application rates.

The compounds of the formula (I) to (V) can be applied directly, i.e. without containing any other components and without having been diluted. In general, it is preferable to apply the compositions to the seed in the form of a suitable formulation. Suitable formulations and methods for seed treatment are known to those skilled in the art. The compounds of the formula (I) to (V) can be converted to the customary formulations relevant to on-seed applications, such as solutions, emulsions, suspensions, powders, foams, slurries or combined with other coating compositions for seed, such as film forming materials, pelleting materials, fine iron or other metal powders, granules, coating material for inactivated seeds, and also ULV formulations.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Usable with preference are alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The formulations for on-seed applications usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also seeds of maize, soybean, rice, oilseed rape, peas, beans, cotton, sunflowers, and beets, or else a wide variety of different vegetable seeds. The formulations usable in accordance with the invention, or the dilute preparations thereof, can also be used for seeds of transgenic plants. In this case, additional synergistic effects may also occur in interaction with the substances formed by expression.

For treatment of seeds with the formulations usable in accordance with the invention, or the preparations prepared therefrom by adding water, all mixing units usable customarily for on-seed applications are useful. Specifically, the procedure in on-seed applications is to place the seeds into a mixer, to add the particular desired amount of the formulations, either as such or after prior dilution with water, and to mix everything until all applied formulations are distributed homogeneously on the seeds. If appropriate, this is followed by a drying operation.

The application rate of the formulations usable in accordance with the invention can be varied within a relatively wide range. It is guided by the particular content of the active ingredients in the formulations and by the seeds. The application rates of each single active ingredient is generally between 0.001 and 15 g per kilogram of seed, preferably between 0.01 and 5 g per kilogram of seed.

GMO

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance.

Plants or plant cultivars (obtained by plant biotechnology methods such as genetic engineering) which may also be treated according to the invention show altered quantity, quality and/or storage-stability of the harvested product and/or altered properties of specific ingredients of the harvested product.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants or plant cultivars (that can be obtained by plant biotechnology methods such as genetic engineering) which may also be treated according to the invention are plants, such as Tobacco plants, with altered post-translational protein modification patterns.

Application Rates

When using the compounds of the formula (I) to (V) as fungicides, the application rates can be varied within a relatively wide range, depending on the kind of application. The application rate of the inventive active ingredients is

in the case of treatment of plant parts, for example leaves: from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used);
in the case of seed treatment: from 0.1 to 200 g per 100 kg of seed, preferably from 1 to 150 g per 100 kg of seed, more preferably from 2.5 to 25 g per 100 kg of seed, even more preferably from 2.5 to 12.5 g per 100 kg of seed;
in the case of soil treatment: from 0.1 to 10 000 g/ha, preferably from 1 to 5000 g/ha.

These application rates are merely by way of example and are not limiting for the purposes of the invention.

### PREPARATION EXAMPLES

In analogy to the examples above and according to the general description of the processes of preparing the compounds according to the invention the compounds in the following Table 1 may be obtained.

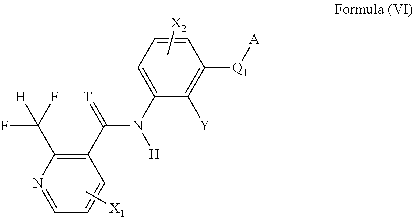

Compounds of formula (VI) represent carboxamides of formula (I) wherein A represents —Ra-Q₂-R

| Ex | | | | | | | |
|---|---|---|---|---|---|---|---|
| N^{o} | X₁ | T | Y | X₂ | Q₁ | A | LogP^{[a]} |
| 1 | 4-CHF₂ | O | Me | H | bond | 2-(2,2-dichlorocyclopropyl)ethyl | 3.92 |
| 2 | H | O | Cl | H | bond | 2,5-difluorophenyl | 3.11 |
| 3 | H | O | Cl | H | bond | phenyl | 3.05 |
| 4 | H | O | Cl | H | bond | 2-fluorophenyl | 2.99 |
| 5 | H | O | Cl | H | bond | 3-fluorophenyl | 3.12 |
| 6 | H | O | Cl | H | bond | 4-fluorophenyl | 3.12 |
| 7 | H | O | Cl | H | bond | 2-chlorophenyl | 3.22 |
| 8 | H | O | Cl | H | bond | 3-chlorophenyl | 3.49 |
| 9 | H | O | Cl | H | bond | 4-chlorophenyl | 3.54 |
| 10 | H | O | Cl | H | bond | 2,4-difluorophenyl | 3.12 |
| 11 | H | O | Me | H | O | sec-butyl | 3.02 |
| 12 | H | O | Me | H | O | heptan-4-yl | 4.26 |
| 13 | H | O | Me | H | O | isobutyl | 3.17 |
| 14 | H | O | Me | H | O | 3-methylbutan-2-yl | 3.41 |
| 15 | H | O | Me | H | O | 4-methylpentan-2-yl | 3.79 |
| 16 | H | O | Me | H | O | cyclohexyl | 3.52 |
| 17 | H | O | Me | H | O | [3-chloro-5-(trifluoromethyl)pyridin-2-yl]methyl | 3.23 |
| 18 | H | O | Me | H | O | 3-chloro-5-(trifluoromethyl)pyridin-2-yl | 3.48 |
| 19 | H | O | Me | H | O | 4-bromobenzyl | 3.55 |
| 20 | H | O | Me | H | O | 1-ethoxypropan-2-yl | 2.57 |
| 21 | H | O | Me | H | O | 1-(prop-2-yn-1-yloxy)propan-2-yl | 2.47 |
| 22 | H | O | Me | H | O | 1-propoxypropan-2-yl | 3.04 |
| 23 | H | O | Me | H | O | 1-isobutoxypropan-2-yl | 3.55 |
| 24 | H | O | Me | H | O | 1-tert-butoxypropan-2-yl | 3.31 |
| 25 | H | O | Me | H | O | 1-(1-chlorocyclopropyl)ethyl | 3.06 |
| 26 | H | O | Me | H | O | 2-cyclopropylethyl | 3.17 |
| 27 | H | O | Me | H | O | 1-(tetrahydrofuran-3-yl)ethyl | 2.26 |
| 28 | H | O | Me | H | O | (1Z)-1-(methoxyimino)propan-2-yl | 2.49 |
| 29 | H | O | Me | H | O | (1Z)-1-(ethoxyimino)propan-2-yl | 2.82 |
| 30 | H | O | Me | H | O | 1-phenoxypropan-2-yl | 3.33 |
| 31 | H | O | Me | H | O | 1-(4-methylphenoxy)propan-2-yl | 3.68 |
| 32 | H | O | Me | H | O | 1-(2-methylphenoxy)propan-2-yl | 3.74 |
| 33 | H | O | Me | H | O | 1-(3-methylphenoxy)propan-2-yl | 3.68 |
| 34 | H | O | Me | H | O | 1-(3-chlorophenoxy)propan-2-yl | 3.76 |
| 35 | H | O | Me | H | O | 1-(2-chlorophenoxy)propan-2-yl | 3.59 |
| 36 | H | O | Me | H | O | 4-fluorobenzyl | 3.06 |
| 37 | H | O | Me | H | O | 2-chlorobenzyl | 3.41 |
| 38 | H | O | Me | H | O | 3-(trifluoromethyl)benzyl | 3.52 |
| 39 | H | O | Me | H | O | 2-(trifluoromethyl)benzyl | 3.55 |
| 40 | H | O | Me | H | O | 3-fluorobenzyl | 3.09 |
| 41 | H | O | Me | H | O | 2-fluorobenzyl | 3.06 |
| 42 | H | O | Me | H | O | 3,5-dichlorobenzyl | 3.96 |
| 43 | H | O | Me | H | O | 2,5-dichlorobenzyl | 3.85 |
| 44 | H | O | Me | H | O | 3,4-dichlorobenzyl | 3.81 |
| 45 | H | O | Me | H | O | 2,3-dichlorobenzyl | 3.83 |
| 46 | H | O | Me | H | O | 2,4-dichlorobenzyl | 3.99 |
| 47 | H | O | Me | H | O | 1-(4-chlorophenyl)ethyl | 3.65 |
| 48 | H | O | Me | H | O | 1-(3,4-dichlorophenyl)ethyl | 4.01 |
| 49 | H | O | Me | H | O | 1-(4-fluorophenyl)ethyl | 3.29 |
| 50 | H | O | Me | H | O | 1-(2-chlorophenyl)ethyl | 3.79 |
| 51 | H | O | Me | H | O | 1-(3-chlorophenyl)ethyl | 3.63 |
| 52 | H | O | Me | H | O | 1-(2,6-difluorophenyl)ethyl | 3.33 |
| 53 | H | O | Me | H | O | 1-[3-(trifluoromethyl)phenyl]ethyl | 3.70 |
| 54 | H | O | Me | H | O | 3-bromobenzyl | 3.52 |
| 55 | H | O | Me | H | O | 1-[2-(trifluoromethyl)phenyl]ethyl | 3.87 |
| 56 | H | O | Me | H | O | 1-(2,5-dichlorophenyl)ethyl | 4.15 |
| 57 | H | O | Me | H | O | 1-(2-fluorophenyl)ethyl | 3.37 |
| 58 | H | O | Me | H | O | 2-[4-(trifluoromethyl)phenyl]ethyl | 3.72 |
| 59 | H | O | Me | H | O | 2-[2-(trifluoromethyl)phenyl]ethyl | 3.74 |
| 60 | H | O | Me | H | O | 2-[3-(trifluoromethyl)phenyl]ethyl | 3.68 |
| 61 | H | O | Me | H | O | 2-(3-fluorophenyl)ethyl | 3.29 |
| 62 | H | O | Me | H | O | 2-(2-fluorophenyl)ethyl | 3.31 |
| 63 | H | O | Me | H | O | 2-(4-fluorophenyl)ethyl | 3.29 |
| 64 | H | O | Me | H | O | 2-(2-chlorophenyl)ethyl | 3.65 |
| 65 | H | O | Me | H | O | 2-(3-chlorophenyl)ethyl | 3.61 |
| 66 | H | O | Me | H | O | 2-(4-chlorophenyl)ethyl | 3.68 |
| 67 | H | O | Me | H | O | 1-(4-chlorophenyl)propan-2-yl | 3.90 |
| 68 | H | O | Me | H | O | 1-(3-chlorophenyl)propan-2-yl | 3.85 |
| 69 | H | O | Me | H | O | 1-(2-chlorophenyl)propan-2-yl | 3.90 |
| 70 | H | O | Me | H | O | 1-(phenylsulfanyl)propan-2-yl | 3.65 |
| 71 | H | O | Me | H | O | 2-thienylmethyl | 2.82 |
| 72 | H | O | Me | H | O | 1-(2-thienyl)propan-2-yl | 3.37 |
| 73 | H | O | Me | H | O | 4-(trifluoromethyl)benzyl | 3.57 |
| 74 | H | O | Me | H | bond | 3,3-dimethylbutyl | 3.92 |
| 75 | H | O | Me | H | O | cyclopentyl | 3.21 |
| 76 | H | O | Me | H | O | 3,3-dichloroprop-2-en-1-yl | 3.21 |
| 77 | H | O | Me | H | O | cyclohexylmethyl | 4.14 |
| 78 | H | O | Me | H | O | 3,3-dimethylbutyl | 3.85 |
| 79 | H | O | Me | H | O | pentan-2-yl | 3.48 |
| 80 | H | O | Me | H | bond | tetrahydro-2H-pyran-2-yl | 2.53 |
| 81 | H | O | Me | H | bond | rel-(1R,4S)-bicyclo[2.2.1]hept-2-yl | 3.80 |
| 82 | H | O | Me | H | bond | tetrahydrofuran-2-yl | 2.02 |
| 83 | H | O | Me | H | O | hexan-3-yl | 3.94 |
| 84 | H | O | Me | H | O | 4,4-dimethylpentan-2-yl | 4.21 |
| 85 | H | O | Me | H | O | 1-cyclopentylethyl | 4.06 |
| 86 | H | O | Me | H | O | hexan-2-yl | 3.94 |
| 87 | H | O | Me | H | O | 5-methylhexan-2-yl | 4.26 |
| 88 | H | O | Me | H | O | 1-phenylpropan-2-yl | 3.62 |
| 89 | H | O | Me | H | O | 1-phenylethyl | 3.33 |
| 90 | H | O | Me | H | bond | isopropoxymethyl | 2.43 |
| 91 | H | O | CF₃ | H | O | cyclopentyl | 3.33 |
| 92 | H | O | CF₃ | H | O | pentan-3-yl | 3.55 |
| 93 | H | O | CF₃ | H | O | hexan-3-yl | 3.92 |
| 94 | H | O | CF₃ | H | O | sec-butyl | 3.17 |
| 95 | 5-Me | O | CF₃ | H | O | sec-butyl | 3.44 |
| 96 | 6-Cl | O | CF₃ | H | O | sec-butyl | 3.83 |
| 97 | 6-Cl | O | CF₃ | H | O | isopropyl | 3.46 |
| 98 | 5-Me | O | CF₃ | H | O | isopropyl | 3.04 |
| 99 | H | O | CF₃ | H | O | isopropyl | 2.80 |
| 100 | H | O | CF₃ | H | O | benzyl | 3.13 |
| 101 | H | O | CF₃ | H | O | 1-phenylethyl | 3.42 |
| 102 | 5-Me | O | CF₃ | H | O | 1-phenylethyl | 3.69 |
| 103 | 6-Cl | O | CF₃ | H | O | 1-phenylethyl | 4.03 |
| 104 | H | O | Me | H | bond | pyrrolidin-1-yl | 0.79 |
| 105 | H | O | Me | H | SO₂ | cyclopentyl | 2.21 |
| 106 | H | O | Me | H | NH | pentan-3-yl | 2.86 |
| 107 | H | O | Me | H | NH | cyclopentyl | 2.17 |
| 108 | H | O | Me | H | bond | N-methylmethanamino | 0.45 |
| 109 | H | O | Me | H | bond | N-methylcyclopentanamino | 1.39 |
| 110 | H | O | Me | H | bond | 5-(trifluoromethyl)pyridin-3-yl | 2.60 |
| 111 | H | O | Me | H | bond | 5-methyl-2-thienyl | 3.19 |
| 112 | H | O | Me | H | bond | 2,4-dimethyl-1,3-thiazol-5-yl | 1.98 |
| 113 | H | O | Me | H | bond | 1-isopropyl-1H-pyrazol-4-yl | 2.17 |
| 114 | H | O | Me | H | bond | 2-isopropyl-1,3-thiazol-4-yl | 2.94 |
| 115 | H | O | Me | H | O | 1-(2,4-dichlorophenyl)ethyl | 4.41 |
| 116 | H | O | Me | H | bond | 2,2-dimethylcyclohexyl | 4.31 |
| 117 | H | O | Me | H | O | 2,3-dimethylbutan-2-yl | 3.61 |
| 118 | H | O | Me | H | O | Et | 2.34 |
| 119 | H | O | Me | H | O | 2,2,2-trifluoroethyl | 2.55 |
| 120 | H | O | Me | H | O | 3,3,3-trifluoropropyl | 2.68 |
| 121 | H | O | Me | H | bond | cyclopentyl | 3.25 |
| 122 | H | O | Me | H | bond | cyclopropyl | 2.43 |
| 123 | 4-CHF₂ | O | Me | H | O | cyclopentyl | 3.70 |

Measurement of LogP values was performed according to EEC directive 79/831 Annex V.A8 by HPLC (High Performance Liquid Chromatography) on reversed phase columns with the following methods:
^{[a]}LogP value is determined by measurement of LC-UV, in an acidic range, with 0.1% formic acid in water and acetonitrile as eluent (linear gradient from 10% acetonitrile to 95% acetonitrile).

Calibration was done with straight-chain alkan2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals.

NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁(intensity₁);δ₂(intensity₂); . . . ;δᵢ(intensityᵢ); . . . ;δₙ(intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

### Example 1

¹H-NMR (300.2 MHz, CDCl₃): δ 8.898 (0.8); 8.882 (0.8); 7.810 (0.6); 7.792 (0.6); 7.648 (0.5); 7.625 (0.6); 7.462 (0.4); 7.274 (0.4); 7.261 (17.8); 7.253 (0.7); 7.249 (1.0); 7.223 (0.5); 7.170 (0.7); 7.148 (0.4); 7.080 (1.1); 6.896 (1.5); 6.714 (0.6); 5.301 (1.5); 2.911 (0.5); 2.894 (0.3); 2.885 (0.3); 2.867 (0.5); 2.293 (4.9); 1.836 (0.5); 1.813 (0.6); 1.618 (0.7); 1.615 (0.6); 1.604 (1.2); 1.597 (0.5); 1.548 (16.0); 1.103 (0.8); 1.101 (0.8); 0.011 (0.3); 0.000 (10.8); −0.011 (0.4)

### Example 2

¹H-NMR (300.2 MHz, CDCl₃): δ 8.847 (5.7); 8.842 (6.4); 8.831 (6.3); 8.826 (6.5); 8.592 (3.4); 8.564 (3.7); 8.523 (0.4); 8.285 (4.4); 8.107 (5.2); 8.082 (5.7); 7.605 (4.8); 7.589 (4.7); 7.579 (4.5); 7.562 (4.2); 7.469 (4.9); 7.442 (9.5); 7.415 (6.2); 7.387 (0.5); 7.320 (0.4); 7.307 (0.4); 7.297 (0.4); 7.280 (0.6); 7.261 (112.3); 7.235 (1.2); 7.222 (7.1); 7.209 (0.8); 7.183 (7.4); 7.178 (8.0); 7.157 (7.3); 7.153 (7.2); 7.141 (4.9); 7.132 (4.0); 7.124 (6.5); 7.115 (7.4); 7.107 (7.1); 7.098 (7.7); 7.093 (5.5); 7.083 (4.0); 7.068 (1.5); 7.049 (3.5); 7.040 (16.0); 7.033 (3.9); 7.029 (3.6); 7.022 (5.3); 7.012 (3.1); 7.002 (3.1); 6.995 (2.5); 6.910 (0.7); 6.858 (6.8); 2.046 (0.5); 1.598 (0.3); 1.586 (0.4); 1.571 (0.6); 1.551 (87.8); 1.260 (0.9); 0.882 (0.6); 0.195 (0.3); 0.019 (0.5); 0.011 (2.6); 0.000 (87.9); −0.011 (4.1); −0.039 (0.4); −0.199 (0.4)

### Example 3

¹H-NMR (400.1 MHz, d₆-DMSO): δ 10.519 (8.0); 8.858 (4.0); 8.855 (4.3); 8.846 (4.2); 8.843 (4.1); 8.227 (2.6); 8.208 (2.8); 7.769 (2.7); 7.756 (3.0); 7.749 (3.3); 7.738 (4.6); 7.720 (3.1); 7.519 (3.5); 7.512 (4.3); 7.498 (9.6); 7.493 (8.0); 7.483 (11.7); 7.473 (4.0); 7.452 (16.0); 7.443 (4.6); 7.436 (8.4); 7.434 (7.4); 7.423 (1.8); 7.419 (1.8); 7.380 (3.4); 7.347 (5.2); 7.344 (5.4); 7.328 (4.3); 7.324 (4.1); 7.245 (7.6); 7.110 (3.7); 3.421 (0.9); 3.369 (1.8); 3.321 (423.9); 3.270 (3.0); 2.682 (0.9); 2.678 (1.2); 2.673 (0.8); 2.608 (0.4); 2.557 (0.9); 2.552 (1.2); 2.548 (1.3); 2.531 (4.0); 2.517 (72.6); 2.513 (144.4); 2.509 (193.4); 2.504 (139.8); 2.500 (67.8); 2.462 (1.2); 2.458 (1.7); 2.453 (1.5); 2.340 (0.9); 2.335 (1.2); 2.331 (0.9)

### Example 4

¹H-NMR (400.1 MHz, d₆-DMSO): δ 10.537 (12.9); 8.855 (6.5); 8.852 (6.9); 8.844 (6.7); 8.840 (6.4); 8.227 (4.2); 8.208 (4.5); 7.803 (4.4); 7.783 (5.1); 7.765 (4.3); 7.753 (4.5); 7.745 (4.3); 7.733 (3.6); 7.549 (2.0); 7.545 (2.4); 7.536 (6.9); 7.531 (5.4); 7.516 (12.7); 7.506 (3.8); 7.497 (7.4); 7.424 (2.5); 7.419 (2.9); 7.405 (6.2); 7.401 (6.3); 7.386 (5.5); 7.379 (7.2); 7.370 (6.3); 7.357 (15.0); 7.355 (15.9); 7.348 (8.8); 7.339 (16.0); 7.323 (4.8); 7.321 (4.7); 7.243 (11.1); 7.108 (5.6); 3.421 (0.5); 3.370 (1.6); 3.321 (506.8); 3.282 (0.8); 3.271 (1.7); 2.682 (1.3); 2.678 (1.7); 2.673 (1.3); 2.517 (107.0); 2.513 (206.0); 2.508 (271.6); 2.504 (196.3); 2.459 (1.5); 2.340 (1.3); 2.335 (1.7); 2.331 (1.2); 1.245 (0.6)

### Example 5

¹H-NMR (400.1 MHz, d₆-DMSO): δ 10.542 (14.4); 8.859 (7.6); 8.855 (8.1); 8.847 (7.9); 8.844 (7.7); 8.225 (4.9); 8.205 (5.3); 7.770 (9.5); 7.750 (10.3); 7.739 (5.2); 7.574 (2.6); 7.570 (1.5); 7.556 (4.8); 7.552 (4.4); 7.539 (5.7); 7.533 (5.6); 7.526 (6.4); 7.518 (4.0); 7.507 (11.3); 7.487 (6.3); 7.377 (16.0); 7.373 (11.8); 7.358 (8.2); 7.354 (7.7); 7.307 (13.9); 7.300 (10.4); 7.297 (11.2); 7.294 (10.2); 7.290 (8.5); 7.284 (14.0); 7.278 (15.5); 7.270 (5.2); 7.242 (13.9); 7.107 (6.8); 3.421 (0.5); 3.369 (2.1); 3.357 (1.2); 3.320 (767.8); 3.270 (2.9); 3.241 (0.3); 3.221 (0.7); 2.682 (1.6); 2.677 (2.3); 2.673 (1.8); 2.609 (0.4); 2.531 (7.9); 2.517 (145.1); 2.513 (288.2); 2.508 (385.4); 2.504 (277.8); 2.500 (134.3); 2.458 (2.3); 2.414 (0.6); 2.357 (0.4); 2.340 (1.8); 2.335 (2.5); 2.331 (1.9); 1.244 (0.8)

### Example 6

¹H-NMR (400.1 MHz, d₆-DMSO): δ 10.528 (7.9); 8.854 (4.4); 8.846 (4.3); 8.842 (4.2); 8.223 (2.7); 8.204 (2.9); 7.768 (2.7); 7.756 (3.3); 7.747 (4.8); 7.737 (3.6); 7.725 (3.3); 7.510 (7.5); 7.491 (10.4); 7.488 (9.5); 7.479 (3.8); 7.474 (8.9); 7.377 (3.3); 7.349 (11.6); 7.332 (7.3); 7.327 (16.0); 7.310 (2.4); 7.305 (5.9); 7.241 (7.3); 7.106 (3.6); 3.421 (0.7); 3.374 (0.4); 3.320 (494.6); 3.272 (2.7); 3.222 (0.3); 2.682 (1.1); 2.678 (1.5); 2.673 (1.1); 2.613 (0.3); 2.609 (0.3); 2.517 (95.5); 2.513 (188.6); 2.508 (252.1); 2.504 (184.4); 2.464 (2.4); 2.460 (2.1); 2.441 (0.4); 2.420 (0.4); 2.340 (1.2); 2.335 (1.7); 2.331 (1.3); 1.244 (0.4)

### Example 7

¹H-NMR (400.1 MHz, d₆-DMSO): δ 10.522 (12.3); 8.847 (6.7); 8.838 (6.7); 8.222 (4.0); 8.204 (4.4); 7.812 (4.2); 7.793 (4.9); 7.756 (3.8); 7.744 (4.4); 7.737 (4.3); 7.726 (3.6); 7.689 (0.5); 7.629 (0.8); 7.620 (6.1); 7.614 (4.8); 7.611 (4.8); 7.606 (4.8); 7.601 (5.7); 7.596 (9.0); 7.588 (2.0); 7.580 (0.9); 7.567 (0.7); 7.526 (4.8); 7.507 (10.0); 7.490 (11.8); 7.485 (16.0); 7.476 (14.8); 7.466 (13.3); 7.463 (11.3); 7.449 (3.8); 7.444 (2.3); 7.405 (0.9); 7.388 (8.2); 7.382 (6.1); 7.377 (9.8); 7.365 (5.8); 7.330 (0.6); 7.311 (0.4); 7.281 (8.3); 7.277 (8.8); 7.262 (7.8); 7.258 (7.6); 7.242 (11.8); 7.107 (5.7); 4.047 (0.6); 4.029 (0.7); 3.420 (0.6); 3.371 (1.1); 3.320 (621.0); 3.271 (3.1); 3.221 (0.5); 3.198 (0.4); 2.687 (0.7); 2.682 (1.4); 2.678 (1.9); 2.673 (1.5); 2.604 (0.4); 2.531 (9.6); 2.518 (112.9); 2.513 (226.1); 2.509 (306.5); 2.504 (226.8); 2.500 (117.4); 2.340 (1.6); 2.336 (2.1); 2.331 (1.6); 1.996 (2.8); 1.244 (1.4); 1.201 (0.8); 1.183 (1.6); 1.165 (0.8); 0.867 (0.4)

### Example 8

¹H-NMR (400.1 MHz, d₆-DMSO): δ 10.545 (8.2); 8.859 (4.9); 8.856 (5.2); 8.847 (5.3); 8.844 (5.0); 8.225 (3.2); 8.206 (3.5); 7.816 (0.7); 7.770 (6.2); 7.751 (7.0); 7.739 (4.3); 7.719 (1.0); 7.628 (0.4); 7.609 (0.6); 7.589 (0.5); 7.558 (1.5); 7.538 (6.5); 7.524 (16.0); 7.508 (9.1); 7.498 (9.6); 7.495 (9.3); 7.489 (5.6); 7.470 (1.4); 7.451 (1.3); 7.445 (1.2); 7.431 (3.8); 7.424 (4.4); 7.421 (3.7); 7.416 (3.3); 7.410 (3.7); 7.405 (2.8); 7.378 (9.5); 7.374 (7.4); 7.359 (5.1); 7.355 (4.9); 7.243 (7.9); 7.107 (3.9); 3.422 (0.5); 3.370 (2.3); 3.321 (838.3); 3.271 (6.7); 3.222 (1.8); 3.211 (0.5); 3.193 (0.4); 3.189 (0.4); 2.682 (1.7); 2.678 (2.3); 2.673 (1.7); 2.518 (142.5); 2.513 (287.8); 2.509 (392.1); 2.504 (295.0); 2.500 (156.0); 2.414 (2.0); 2.340 (2.1); 2.336 (2.8); 2.331 (2.3); 1.996 (0.6); 1.245 (1.0); 0.867 (0.5)

### Example 9

¹H-NMR (400.1 MHz, d₆-DMSO): δ 10.538 (6.9); 8.854 (4.4); 8.845 (4.4); 8.224 (2.8); 8.205 (2.9); 7.816 (0.6); 7.802 (0.8); 7.796 (0.8); 7.780 (0.9); 7.768 (3.3); 7.756 (5.6); 7.737 (5.4); 7.573 (10.0); 7.552 (16.0); 7.521 (3.0); 7.502 (6.0); 7.484 (15.9); 7.463 (8.7); 7.438 (0.5); 7.396 (0.4); 7.377 (3.2); 7.355 (4.8); 7.352 (4.9); 7.336 (4.0); 7.333 (3.8); 7.242 (6.5); 7.107 (3.2); 3.421 (0.8); 3.398 (0.3); 3.372 (3.3); 3.320 (704.1); 3.271 (2.2); 3.219 (0.5); 2.682 (1.6); 2.678 (2.2); 2.609 (0.6); 2.605 (0.5); 2.564 (1.4); 2.560 (1.4); 2.513 (269.4); 2.508 (356.7); 2.504 (261.0); 2.402 (0.4); 2.391 (0.4); 2.374 (0.4); 2.335 (2.3); 1.243 (0.6)

### Example 10

¹H-NMR (400.1 MHz, d₆-DMSO): δ 10.544 (13.1); 8.855 (7.2); 8.852 (7.6); 8.844 (7.7); 8.840 (7.5); 8.225 (4.8); 8.206 (5.2); 7.834 (0.5); 7.810 (4.9); 7.790 (5.7); 7.764 (4.8); 7.753 (5.2); 7.745 (5.1); 7.733 (4.4); 7.538 (5.5); 7.519 (11.0); 7.499 (7.4); 7.482 (7.2); 7.465 (7.0); 7.461 (5.0); 7.444 (4.5); 7.440 (4.8); 7.434 (4.4); 7.415 (6.1); 7.409 (6.1); 7.391 (4.1); 7.384 (4.4); 7.376 (6.2); 7.359 (9.0); 7.355 (9.3); 7.340 (7.7); 7.336 (7.4); 7.258 (3.8); 7.252 (4.0); 7.240 (16.0); 7.232 (7.1); 7.216 (3.5); 7.211 (3.1); 7.105 (5.9); 3.719 (0.4); 3.438 (0.4); 3.420 (3.4); 3.374 (0.6); 3.320 (1067.3); 3.270 (12.0); 3.208 (0.9); 3.194 (0.8); 3.149 (0.5); 3.112 (0.3); 2.682 (2.5); 2.678 (3.4); 2.673 (2.7); 2.613 (1.2); 2.609 (1.4); 2.604 (1.0); 2.518 (203.2); 2.513 (408.3); 2.509 (554.7); 2.504 (415.8); 2.500 (218.7); 2.464 (15.1); 2.459 (14.0); 2.402 (2.0); 2.340 (3.1); 2.336 (4.1); 2.331 (3.3); 2.310 (0.8); 2.191 (0.4); 2.161 (0.4); 1.246 (0.7)

### Example 11

¹H-NMR (400.0 MHz, d₆-DMSO): δ 10.161 (3.1); 8.826 (1.8); 8.824 (1.9); 8.814 (1.9); 8.812 (1.8); 8.211 (1.7); 8.191 (1.8); 7.743 (1.3); 7.731 (1.3); 7.724 (1.2); 7.712 (1.1); 7.337 (1.4); 7.201 (3.0); 7.179 (1.0); 7.159 (2.4); 7.139 (1.5); 7.066 (1.5); 7.017 (2.5); 6.998 (1.9); 6.900 (2.3); 6.879 (1.9); 4.419 (0.8); 4.404 (1.5); 4.389 (1.6); 4.374 (0.8); 3.332 (241.5); 2.891 (1.8); 2.731 (1.5); 2.676 (0.5); 2.671 (0.7); 2.667 (0.5); 2.541 (0.4); 2.524 (2.1); 2.511 (44.3); 2.507 (88.8); 2.502 (116.4); 2.498 (83.9); 2.333 (0.5); 2.329 (0.7); 2.324 (0.5); 2.081 (16.0); 1.721 (0.3); 1.705 (0.6); 1.687 (1.2); 1.669 (1.4); 1.652 (1.7); 1.634 (1.3); 1.619 (1.2); 1.601 (0.6); 1.585 (0.3); 1.252 (9.5); 1.237 (9.8); 0.966 (4.7); 0.948 (9.7); 0.929 (4.3); 0.008 (0.8); 0.000 (23.5); −0.008 (0.9)

### Example 12

¹H-NMR (400.0 MHz, d₆-DMSO): δ 10.151 (2.8); 8.826 (1.5); 8.823 (1.5); 8.814 (1.5); 8.811 (1.5); 8.317 (0.4); 8.210 (1.4); 8.191 (1.5); 7.743 (1.0); 7.731 (1.1); 7.724 (1.0); 7.712 (0.9); 7.337 (1.1); 7.202 (2.5); 7.174 (0.8); 7.154 (1.9); 7.134 (1.2); 7.067 (1.2); 7.004 (2.0); 6.984 (1.6); 6.889 (1.8); 6.868 (1.5); 4.391 (0.8); 4.377 (1.2); 4.362 (0.8); 3.330 (187.1); 2.891 (1.8); 2.731 (1.5); 2.675 (0.5); 2.671 (0.7); 2.667 (0.5); 2.541 (0.4); 2.524 (2.0); 2.511 (41.2); 2.506 (82.8); 2.502 (108.6); 2.497 (77.8); 2.493 (37.3); 2.333 (0.5); 2.329 (0.7); 2.324 (0.5); 2.075 (13.0); 1.652 (0.4); 1.633 (0.9); 1.618 (2.2); 1.608 (1.7); 1.602 (2.5); 1.594 (2.9); 1.579 (2.5); 1.566 (1.3); 1.546 (0.5); 1.467 (0.4); 1.450 (0.6); 1.433 (1.1); 1.412 (1.3); 1.393 (1.7); 1.376 (1.7); 1.358 (1.4); 1.351 (1.0); 1.341 (0.8); 1.334 (0.9); 1.324 (0.5); 1.317 (0.6); 1.235 (0.4); 0.911 (7.9); 0.893 (16.0); 0.875 (6.9); 0.008 (1.1); 0.000 (31.1); −0.008 (1.2)

### Example 13

¹H-NMR (400.0 MHz, d₆-DMSO): δ 10.192 (2.8); 8.825 (1.7); 8.813 (1.7); 8.217 (1.5); 8.197 (1.6); 7.745 (1.1); 7.733 (1.2); 7.725 (1.1); 7.713 (1.0); 7.337 (1.2); 7.202 (2.6); 7.188 (0.9); 7.167 (2.1); 7.147 (1.3); 7.066 (1.3); 7.029 (2.2); 7.009 (1.7); 6.873 (2.0); 6.852 (1.8); 3.778 (4.4); 3.763 (4.5); 3.332 (190.6); 2.890 (1.4); 2.731 (1.2); 2.671 (0.7); 2.506 (77.9); 2.502 (103.5); 2.498 (79.1); 2.329 (0.7); 2.107 (13.5); 2.077 (1.0); 2.060 (1.2); 2.044 (0.9); 2.028 (0.5); 1.234 (0.4); 1.027 (16.0); 1.010 (15.6); 0.008 (0.7); 0.000 (18.9)

### Example 14

¹H-NMR (400.0 MHz, d₆-DMSO): δ 10.160 (3.2); 8.827 (1.8); 8.824 (1.9); 8.815 (1.9); 8.812 (1.8); 8.211 (1.7); 8.191 (1.8); 7.744 (1.3); 7.732 (1.3); 7.725 (1.3); 7.713 (1.1); 7.337 (1.4); 7.202 (3.0); 7.175 (1.0); 7.155 (2.4); 7.135 (1.5); 7.066 (1.5); 7.008 (2.5); 6.988 (2.0); 6.896 (2.2); 6.875 (1.9); 4.299 (1.2); 4.285 (1.6); 4.271 (1.2); 4.256 (0.3); 3.365 (0.5); 3.334 (179.5); 2.891 (1.6); 2.732 (1.4); 2.676 (0.4); 2.671 (0.5); 2.667 (0.4); 2.524 (1.4); 2.511 (30.1); 2.507 (60.0); 2.502 (77.8); 2.498 (55.3); 2.493 (26.6); 2.333 (0.4); 2.329 (0.5); 2.324 (0.4); 2.088 (16.0); 1.933 (0.7); 1.916 (1.0); 1.903 (1.1); 1.886 (0.8); 1.235 (0.3); 1.191 (9.3); 1.175 (9.2); 0.995 (9.1); 0.978 (8.9); 0.966 (9.4); 0.949 (8.8); 0.000 (9.5); −0.009 (0.4)

### Example 15

¹H-NMR (400.0 MHz, d₆-DMSO): δ 10.156 (3.3); 8.826 (1.9); 8.823 (1.9); 8.814 (2.0); 8.209 (1.7); 8.190 (1.9); 7.952 (1.2); 7.743 (1.3); 7.731 (1.4); 7.724 (1.3); 7.711 (1.2); 7.336 (1.4); 7.201 (3.1); 7.185 (1.1); 7.164 (2.4); 7.144 (1.5); 7.065 (1.5); 7.013 (2.5); 6.993 (2.0); 6.918 (2.3); 6.897 (2.0); 4.539 (0.6); 4.525 (1.0); 4.506 (1.0); 4.492 (0.6); 3.334 (312.1); 2.891 (8.7); 2.732 (7.3); 2.676 (0.5); 2.671 (0.7); 2.667 (0.5); 2.541 (0.4); 2.524 (2.0); 2.507 (90.9); 2.502 (116.6); 2.498 (83.4); 2.493 (40.2); 2.333 (0.6); 2.329 (0.7); 2.324 (0.5); 2.068 (16.0); 1.814 (0.4); 1.798 (0.8); 1.781 (1.1); 1.764 (1.0); 1.747 (0.6); 1.700 (0.7); 1.681 (0.9); 1.665 (1.3); 1.647 (1.1); 1.630 (0.7); 1.433 (0.9); 1.421 (1.0); 1.414 (0.9); 1.401 (1.3); 1.387 (0.8); 1.380 (0.7); 1.368 (0.6); 1.247 (9.0); 1.232 (9.2); 0.940 (9.9); 0.923 (9.6); 0.898 (9.6); 0.881 (9.3); 0.008 (0.5); 0.000 (14.0); −0.009 (0.6)

### Example 16

¹H-NMR (400.0 MHz, d₆-DMSO): δ 10.157 (3.4); 8.823 (1.9); 8.814 (1.9); 8.210 (1.7); 8.191 (1.9); 7.743 (1.3); 7.731 (1.4); 7.724 (1.3); 7.712 (1.2); 7.337 (1.4); 7.201 (3.0); 7.171 (1.0); 7.152 (2.4); 7.131 (1.6); 7.066 (1.5); 7.022 (2.5); 7.002 (1.9); 6.923 (2.3); 6.902 (1.9); 4.391 (0.6); 4.381 (0.8); 4.372 (1.2); 4.362 (0.9); 4.351 (0.6); 4.344 (0.3); 3.333 (288.8); 2.891 (1.9); 2.731 (1.6); 2.675 (0.6); 2.671 (0.8); 2.667 (0.6); 2.506 (95.5); 2.502 (124.2); 2.498 (90.9); 2.333 (0.6); 2.329 (0.8); 2.324 (0.6); 2.095 (16.0); 1.909 (1.1); 1.890 (1.4); 1.879 (1.4); 1.871 (1.3); 1.717 (1.3); 1.701 (1.5); 1.526 (2.1); 1.504 (2.2); 1.482 (1.0); 1.440 (0.5); 1.431 (0.7); 1.408 (1.5); 1.384 (1.5); 1.378 (1.5); 1.359 (1.7); 1.335 (0.8); 1.311 (0.5); 1.235 (0.5); 0.008 (0.5); 0.000 (14.5); −0.008 (0.6)

### Example 17

¹H-NMR (400.1 MHz, d₆-DMSO): δ 10.189 (3.6); 9.000 (3.2); 8.998 (3.1); 8.829 (1.8); 8.820 (1.8); 8.572 (3.3); 8.568 (3.2); 8.222 (1.5); 8.202 (1.7); 7.747 (1.2); 7.735 (1.3); 7.728 (1.2); 7.715 (1.0); 7.339 (1.4); 7.214 (1.0); 7.203 (3.4); 7.195 (2.2); 7.174 (1.5); 7.102 (2.4); 7.083 (1.6); 7.068 (1.6); 7.014 (2.2); 6.993 (1.8); 5.415 (8.3); 4.047 (0.4); 4.029 (0.4); 3.308 (31.2); 2.681 (0.5); 2.677 (0.7); 2.672 (0.5); 2.530 (1.8); 2.517 (46.5); 2.512 (95.1); 2.508 (129.1); 2.503 (91.0); 2.499 (42.8); 2.463 (1.1); 2.458 (1.2); 2.454 (0.8); 2.339 (0.6); 2.335 (0.8); 2.330 (0.6); 2.116 (16.0); 1.995 (1.9); 1.201 (0.5); 1.183 (1.0); 1.165 (0.5)

### Example 18

¹H-NMR (400.1 MHz, d₆-DMSO): δ 18.915 (0.4); 18.179 (0.4); 10.307 (3.5); 10.291 (0.5); 10.142 (0.4); 8.837 (2.2); 8.825 (2.0); 8.601 (3.3); 8.596 (3.6); 8.585 (0.6); 8.507 (3.0); 8.480 (0.4); 8.244 (1.6); 8.229 (1.7); 8.207 (0.5); 7.757 (1.5); 7.746 (1.5); 7.740 (1.4); 7.726 (1.3); 7.521 (0.3); 7.474 (1.7); 7.455 (2.4); 7.416 (0.4); 7.388 (0.4); 7.365 (1.6); 7.355 (2.2); 7.345 (2.3); 7.325 (1.2); 7.294 (0.4); 7.277 (0.4); 7.245 (0.6); 7.219 (3.7); 7.206 (0.6); 7.186 (0.6); 7.150 (2.1); 7.131 (1.9); 7.084 (1.7); 4.529 (0.4); 3.412 (3.2); 3.376 (0.7); 3.362 (8.8); 3.312 (1747.4); 3.262 (15.8); 3.212 (2.3); 3.189 (0.7); 2.686 (1.3); 2.682 (2.9); 2.677 (4.4); 2.672 (3.3); 2.608 (1.2); 2.563 (2.2); 2.558 (3.2); 2.553 (2.7); 2.549 (1.8); 2.530 (15.4); 2.526 (24.7); 2.517 (258.9); 2.513 (529.4); 2.508 (729.4); 2.504 (527.7); 2.499 (266.8); 2.463 (13.7); 2.458 (13.2); 2.453 (10.3); 2.413 (2.3); 2.344 (2.2); 2.340 (3.7); 2.335 (5.0); 2.330 (3.7); 2.281 (2.5); 2.196 (0.7); 2.150 (0.4); 2.122 (0.4); 2.115 (0.3); 2.073 (1.3); 2.064 (0.6); 2.038 (16.0); 2.007 (0.7); 1.995 (0.8); 1.955 (1.9); 1.889 (0.4); 1.815 (0.4); 1.289 (0.4); 1.245 (1.1); 1.183 (0.4); 0.867 (0.9); −3.471 (0.4)

### Example 19

¹H-NMR (400.0 MHz, d₆-DMSO): δ 10.215 (3.9); 8.827 (2.4); 8.815 (2.4); 8.222 (2.1); 8.203 (2.2); 7.745 (1.5); 7.733 (1.6); 7.726 (1.6); 7.714 (1.4); 7.621 (5.3); 7.600 (7.1); 7.572 (1.6); 7.454 (5.7); 7.433 (4.7); 7.406 (1.5); 7.385 (1.2); 7.342 (1.4); 7.206 (3.8); 7.184 (2.6); 7.164 (1.8); 7.075 (3.2); 7.056 (2.2); 6.966 (2.6); 6.946 (2.2); 6.818 (0.3); 6.798 (0.7); 6.778 (0.4); 6.288 (0.7); 6.268 (0.6); 6.246 (0.7); 6.226 (0.6); 5.138 (9.3); 4.996 (2.4); 4.822 (1.4); 3.342 (92.0); 2.889 (1.5); 2.730 (1.3); 2.502 (36.8); 2.143 (16.0); 1.945 (4.2); 0.000 (1.9)

### Example 20

¹H-NMR (601.6 MHz, d₆-DMSO): δ 10.168 (3.6); 8.825 (1.9); 8.823 (2.0); 8.817 (2.0); 8.815 (2.0); 8.210 (1.9); 8.197 (2.0); 7.952 (0.5); 7.739 (1.4); 7.731 (1.5); 7.726 (1.4); 7.718 (1.3); 7.292 (1.3); 7.202 (2.9); 7.176 (1.2); 7.163 (2.6); 7.150 (1.6); 7.112 (1.4); 7.040 (2.6); 7.027 (2.1); 6.954 (2.4); 6.940 (2.1); 4.574 (0.8); 4.564 (1.3); 4.556 (1.3); 4.546 (0.8); 3.574 (1.2); 3.564 (1.3); 3.557 (2.1); 3.547 (2.0); 3.519 (0.4); 3.515 (1.1); 3.512 (1.3); 3.508 (2.5); 3.501 (5.2); 3.492 (4.4); 3.490 (4.0); 3.484 (1.8); 3.480 (1.4); 3.478 (1.4); 3.474 (0.5); 3.330 (81.9); 3.014 (0.4); 2.890 (3.6); 2.761 (0.3); 2.730 (2.9); 2.616 (0.4); 2.613 (0.5); 2.610 (0.4); 2.522 (0.9); 2.519 (1.1); 2.516 (1.1); 2.504 (57.3); 2.501 (78.5); 2.498 (59.3); 2.496 (30.0); 2.389 (0.4); 2.386 (0.5); 2.383 (0.4); 2.128 (0.3); 2.083 (16.0); 1.249 (9.4); 1.239 (9.6); 1.220 (0.5); 1.215 (0.4); 1.209 (0.4); 1.179 (0.4); 1.169 (0.3); 1.124 (6.2); 1.112 (12.4); 1.101 (6.2); 1.087 (0.3); 1.076 (0.4); 0.000 (7.7); −0.006 (0.3)

### Example 21

¹H-NMR (400.0 MHz, d₆-DMSO): δ 10.168 (3.6); 8.824 (2.0); 8.815 (2.0); 8.812 (2.0); 8.213 (1.8); 8.193 (1.9); 7.743 (1.3); 7.731 (1.4); 7.723 (1.4); 7.711 (1.2); 7.338 (1.5); 7.202 (3.3); 7.187 (1.1); 7.166 (2.4); 7.146 (1.6); 7.067 (1.7); 7.051 (2.6); 7.031 (1.9); 6.956 (2.3); 6.936 (2.0); 4.622 (0.7); 4.607 (1.2); 4.596 (1.2); 4.581 (0.7); 4.215 (8.3); 4.209 (8.6); 3.668 (1.0); 3.653 (0.9); 3.642 (2.3); 3.627 (2.3); 3.610 (2.2); 3.600 (2.4); 3.585 (1.0); 3.574 (0.9); 3.459 (2.2); 3.453 (4.6); 3.448 (2.3); 3.335 (105.8); 2.890 (1.8); 2.730 (1.5); 2.524 (0.7); 2.510 (17.4); 2.506 (35.9); 2.501 (47.9); 2.497 (35.8); 2.493 (18.2); 2.085 (16.0); 1.259 (9.0); 1.244 (9.1); 0.000 (0.9)

### Example 22

¹H-NMR (601.6 MHz, d₆-DMSO): δ 10.166 (3.6); 8.824 (1.9); 8.822 (2.0); 8.817 (2.0); 8.814 (2.0); 8.210 (1.9); 8.197 (1.9); 7.952 (0.6); 7.738 (1.4); 7.730 (1.5); 7.725 (1.4); 7.717 (1.3); 7.292 (1.3); 7.201 (2.9); 7.175 (1.1); 7.162 (2.6); 7.148 (1.6); 7.111 (1.4); 7.039 (2.5); 7.026 (2.1); 6.956 (2.4); 6.942 (2.1); 4.579 (0.7); 4.569 (1.2); 4.561 (1.2); 4.551 (0.8); 3.575 (1.2); 3.565 (1.2); 3.557 (2.1); 3.548 (2.0); 3.509 (2.0); 3.502 (2.1); 3.492 (1.3); 3.485 (1.3); 3.424 (0.5); 3.419 (1.8); 3.415 (1.9); 3.408 (3.8); 3.404 (3.8); 3.397 (2.1); 3.394 (2.0); 3.389 (0.7); 3.329 (54.2); 3.015 (0.4); 2.890 (4.7); 2.761 (0.4); 2.730 (3.7); 2.616 (0.4); 2.613 (0.5); 2.610 (0.4); 2.522 (0.9); 2.519 (1.1); 2.516 (1.1); 2.507 (26.4); 2.504 (56.1); 2.501 (77.2); 2.498 (57.8); 2.496 (28.7); 2.389 (0.4); 2.386 (0.5); 2.383 (0.4); 2.084 (16.0); 1.538 (0.5); 1.526 (1.8); 1.515 (3.6); 1.503 (3.9); 1.492 (2.0); 1.480 (0.6); 1.253 (9.4); 1.243 (9.6); 1.220 (0.5); 1.209 (0.4); 1.199 (0.3); 1.183 (0.4); 1.173 (0.4); 0.871 (6.5); 0.858 (12.8); 0.846 (6.2); 0.829 (0.4); 0.000 (8.6); −0.006 (0.4)

### Example 23

¹H-NMR (601.6 MHz, d₆-DMSO): δ 10.164 (3.7); 8.824 (2.0); 8.822 (2.1); 8.816 (2.0); 8.814 (1.9); 8.209 (1.9); 8.197 (1.9); 7.953 (1.0); 7.737 (1.4); 7.729 (1.5); 7.725 (1.4); 7.717 (1.3); 7.291 (1.3); 7.201 (2.9); 7.175 (1.2); 7.161 (2.6); 7.148 (1.6); 7.111 (1.4); 7.039 (2.6); 7.026 (2.2); 6.958 (2.4); 6.945 (2.1); 4.584 (0.7); 4.574 (1.2); 4.566 (1.2); 4.556 (0.8); 3.575 (1.2); 3.566 (1.2); 3.558 (2.0); 3.548 (1.9); 3.513 (1.9); 3.506 (2.1); 3.495 (1.3); 3.489 (1.2); 3.329 (55.1); 3.246 (0.5); 3.230 (3.4); 3.226 (3.5); 3.219 (3.6); 3.215 (3.5); 3.200 (0.5); 3.015 (0.4); 2.890 (7.7); 2.761 (0.4); 2.730 (6.2); 2.616 (0.4); 2.613 (0.6); 2.610 (0.4); 2.522 (0.9); 2.519 (1.2); 2.516 (1.3); 2.504 (58.2); 2.501 (78.7); 2.499 (58.9); 2.389 (0.4); 2.386 (0.5); 2.383 (0.4); 2.128 (0.4); 2.085 (16.0); 1.934 (0.6); 1.816 (0.7); 1.805 (1.4); 1.794 (1.8); 1.783 (1.5); 1.771 (0.8); 1.257 (9.1); 1.247 (9.3); 1.229 (0.6); 1.220 (0.6); 1.209 (0.5); 1.187 (0.5); 1.176 (0.5); 0.858 (12.6); 0.853 (13.1); 0.847 (12.9); 0.842 (12.8); 0.829 (0.7); 0.824 (0.6); 0.818 (0.5); 0.813 (0.4); 0.000 (7.5); −0.006 (0.3)

### Example 24

¹H-NMR (601.6 MHz, d₆-DMSO): δ 10.164 (0.9); 8.824 (0.5); 8.822 (0.5); 8.816 (0.5); 8.814 (0.5); 8.210 (0.5); 8.197 (0.5); 7.952 (0.4); 7.738 (0.4); 7.730 (0.4); 7.725 (0.4); 7.717 (0.3); 7.202 (0.7); 7.156 (0.7); 7.142 (0.4); 7.112 (0.4); 7.027 (0.7); 7.014 (0.6); 6.939 (0.6); 6.925 (0.6); 4.450 (0.4); 4.441 (0.4); 3.527 (0.3); 3.517 (0.4); 3.511 (0.5); 3.501 (0.4); 3.409 (0.5); 3.402 (0.5); 3.393 (0.4); 3.386 (0.4); 3.329 (12.9); 2.890 (3.1); 2.730 (2.5); 2.519 (0.4); 2.516 (0.6); 2.504 (15.3); 2.501 (20.4); 2.498 (15.2); 2.085 (4.0); 1.259 (2.3); 1.248 (2.4); 1.147 (16.0); 0.000 (2.3)

### Example 25

¹H-NMR (400.0 MHz, d₆-DMSO): δ 10.171 (3.6); 8.826 (2.0); 8.817 (2.0); 8.814 (2.0); 8.217 (1.8); 8.198 (1.9); 7.952 (1.2); 7.746 (1.3); 7.734 (1.4); 7.727 (1.3); 7.715 (1.2); 7.341 (1.4); 7.206 (3.1); 7.187 (1.0); 7.167 (2.4); 7.147 (1.7); 7.091 (2.7); 7.071 (3.2); 6.971 (2.3); 6.951 (1.9); 4.287 (0.7); 4.272 (2.1); 4.257 (2.1); 4.242 (0.7); 3.338 (169.6); 3.308 (0.4); 2.890 (8.4); 2.731 (7.3); 2.671 (0.3); 2.524 (0.8); 2.510 (19.5); 2.506 (39.9); 2.502 (53.3); 2.497 (40.2); 2.329 (0.3); 2.139 (16.0); 1.387 (9.2); 1.372 (9.2); 1.106 (0.6); 1.098 (0.7); 1.087 (1.1); 1.076 (2.3); 1.063 (0.8); 1.056 (1.5); 1.050 (2.7); 1.040 (3.1); 1.032 (2.5); 1.020 (6.0); 1.002 (0.7); 0.000 (0.9)

### Example 26

¹H-NMR (400.0 MHz, d₆-DMSO): δ 10.191 (3.3); 8.824 (2.2); 8.815 (2.0); 8.812 (2.0); 8.217 (1.8); 8.198 (1.9); 7.952 (0.6); 7.743 (1.5); 7.731 (1.5); 7.724 (1.4); 7.712 (1.2); 7.338 (1.5); 7.202 (3.6); 7.177 (2.5); 7.157 (1.5); 7.067 (1.7); 7.033 (2.6); 7.014 (2.0); 6.902 (2.3); 6.882 (2.0); 4.059 (2.4); 4.043 (5.1); 4.028 (2.5); 3.373 (0.7); 3.342 (175.0); 2.890 (4.3); 2.731 (3.7); 2.671 (0.3); 2.524 (0.8); 2.511 (19.4); 2.506 (39.2); 2.502 (51.8); 2.498 (38.4); 2.493 (19.2); 2.178 (1.5); 2.101 (16.0); 1.683 (1.3); 1.667 (3.7); 1.650 (3.7); 1.634 (1.4); 1.616 (0.3); 1.222 (1.3); 1.206 (1.4); 0.908 (0.3); 0.895 (0.7); 0.889 (0.6); 0.877 (1.1); 0.865 (0.7); 0.858 (0.8); 0.846 (0.5); 0.840 (0.4); 0.477 (1.0); 0.466 (3.1); 0.462 (3.3); 0.457 (1.5); 0.452 (1.6); 0.446 (3.2); 0.442 (3.2); 0.432 (1.5); 0.415 (0.5); 0.162 (1.1); 0.149 (3.9); 0.137 (3.6); 0.126 (1.0); 0.100 (0.5); 0.088 (0.5); 0.000 (0.5)

### Example 27

¹H-NMR (400.0 MHz, d₆-DMSO): δ 10.366 (0.9); 10.170 (4.4); 9.000 (0.5); 8.996 (0.6); 8.988 (0.6); 8.984 (0.6); 8.839 (0.6); 8.827 (3.2); 8.824 (3.1); 8.816 (2.9); 8.812 (2.8); 8.687 (0.5); 8.669 (0.5); 8.265 (0.4); 8.245 (0.5); 8.211 (2.5); 8.192 (2.7); 7.952 (1.5); 7.863 (0.4); 7.851 (0.4); 7.843 (0.4); 7.831 (0.4); 7.763 (0.3); 7.744 (2.2); 7.732 (2.2); 7.725 (2.0); 7.713 (1.7); 7.626 (0.4); 7.491 (1.2); 7.471 (0.6); 7.397 (0.3); 7.376 (0.6); 7.362 (0.5); 7.356 (0.8); 7.336 (2.1); 7.285 (0.6); 7.266 (0.4); 7.227 (0.9); 7.201 (4.7); 7.187 (1.6); 7.166 (3.6); 7.146 (2.3); 7.092 (0.4); 7.065 (2.3); 7.031 (3.8); 7.011 (2.9); 6.924 (3.2); 6.903 (2.7); 4.432 (0.8); 4.416 (1.8); 4.401 (1.7); 4.381 (1.1); 3.871 (1.4); 3.851 (2.5); 3.831 (1.7); 3.812 (0.8); 3.802 (0.7); 3.791 (2.4); 3.781 (2.1); 3.771 (2.9); 3.761 (1.9); 3.750 (1.2); 3.740 (0.6); 3.695 (0.6); 3.689 (1.1); 3.677 (1.5); 3.670 (2.5); 3.656 (1.3); 3.650 (2.1); 3.638 (0.5); 3.631 (0.8); 3.575 (1.6); 3.558 (1.9); 3.554 (1.8); 3.537 (1.5); 3.510 (0.9); 3.492 (1.1); 3.471 (0.8); 3.347 (355.4); 3.291 (0.4); 2.890 (11.0); 2.731 (9.3); 2.676 (0.4); 2.672 (0.6); 2.667 (0.4); 2.542 (0.9); 2.524 (2.6); 2.520 (3.3); 2.511 (33.6); 2.507 (68.5); 2.502 (91.2); 2.498 (66.7); 2.494 (33.1); 2.466 (0.4); 2.334 (0.4); 2.329 (0.6); 2.325 (0.5); 2.137 (3.7); 2.083 (8.7); 2.068 (16.0); 2.039 (0.6); 2.027 (0.4); 2.017 (0.8); 2.005 (0.7); 1.999 (0.7); 1.986 (1.1); 1.975 (0.8); 1.965 (0.9); 1.956 (0.6); 1.946 (0.5); 1.934 (0.4); 1.891 (0.3); 1.814 (0.6); 1.795 (0.6); 1.783 (0.5); 1.764 (0.4); 1.702 (0.4); 1.682 (1.1); 1.671 (0.5); 1.663 (1.1); 1.652 (1.0); 1.643 (0.5); 1.632 (0.9); 1.613 (0.3); 1.257 (8.8); 1.242 (8.8); 1.227 (4.9); 1.212 (4.8)

### Example 28

¹H-NMR (400.0 MHz, d₆-DMSO): δ 10.301 (0.9); 10.209 (1.0); 10.193 (2.1); 8.825 (2.3); 8.816 (1.8); 8.262 (0.6); 8.243 (0.7); 8.217 (1.5); 8.197 (1.6); 7.952 (2.3); 7.757 (0.6); 7.744 (1.5); 7.732 (1.3); 7.725 (1.5); 7.713 (1.0); 7.493 (2.2); 7.477 (2.3); 7.366 (1.1); 7.345 (1.1); 7.336 (1.3); 7.246 (1.1); 7.231 (1.1); 7.226 (1.1); 7.200 (3.3); 7.178 (2.0); 7.158 (1.4); 7.088 (2.3); 7.066 (2.3); 6.969 (1.4); 6.949 (1.2); 6.883 (0.8); 6.868 (0.9); 6.717 (0.5); 6.697 (0.5); 6.577 (1.2); 6.575 (1.2); 5.394 (0.4); 5.378 (0.5); 5.362 (0.4); 5.025 (0.8); 5.009 (1.3); 4.993 (0.9); 3.878 (5.5); 3.764 (16.0); 3.348 (260.2); 3.309 (0.5); 2.890 (15.8); 2.731 (13.7); 2.671 (0.4); 2.525 (1.1); 2.511 (25.2); 2.507 (51.1); 2.503 (67.6); 2.498 (50.1); 2.466 (4.2); 2.375 (5.1); 2.329 (0.5); 2.325 (0.4); 2.120 (4.2); 2.112 (1.6); 2.097 (9.7); 1.846 (0.9); 1.471 (5.5); 1.455 (5.6); 1.446 (2.7); 1.430 (2.4); 1.299 (0.5); 1.283 (0.6); 1.278 (0.4); 1.262 (0.4); 1.236 (0.6); 1.222 (0.6); 1.173 (0.7); 1.152 (1.0); 1.136 (0.8); 1.077 (0.5); 1.064 (0.6); 1.047 (0.6); 1.031 (0.5); 0.000 (0.5)

### Example 29

¹H-NMR (400.0 MHz, d₆-DMSO): δ 10.209 (1.0); 10.192 (1.9); 8.837 (0.4); 8.826 (1.7); 8.816 (1.8); 8.216 (1.4); 8.197 (1.5); 7.952 (2.3); 7.744 (1.2); 7.732 (1.1); 7.725 (1.1); 7.713 (1.0); 7.468 (2.0); 7.452 (2.1); 7.335 (1.2); 7.238 (0.4); 7.200 (2.6); 7.193 (1.0); 7.172 (1.9); 7.152 (1.3); 7.084 (2.1); 7.064 (2.6); 6.972 (1.3); 6.952 (1.1); 6.868 (0.9); 6.853 (0.9); 6.720 (0.6); 6.700 (0.5); 6.545 (1.0); 5.398 (0.4); 5.382 (0.5); 5.366 (0.3); 5.025 (0.7); 5.008 (1.1); 4.992 (0.8); 4.160 (0.3); 4.143 (1.0); 4.126 (1.0); 4.109 (0.4); 4.051 (1.1); 4.033 (3.3); 4.015 (3.4); 3.998 (1.2); 3.350 (325.6); 2.891 (16.0); 2.731 (13.6); 2.677 (0.4); 2.672 (0.5); 2.668 (0.4); 2.525 (1.2); 2.512 (28.6); 2.507 (58.4); 2.503 (77.7); 2.498 (57.7); 2.494 (29.1); 2.334 (0.4); 2.330 (0.5); 2.325 (0.5); 2.231 (0.3); 2.157 (1.7); 2.122 (4.2); 2.110 (0.8); 2.097 (8.6); 2.074 (0.4); 1.935 (0.3); 1.920 (0.6); 1.906 (0.5); 1.474 (4.8); 1.458 (5.0); 1.452 (3.0); 1.436 (2.4); 1.257 (1.6); 1.240 (3.4); 1.222 (2.1); 1.209 (1.0); 1.189 (0.6); 1.173 (3.9); 1.156 (7.4); 1.138 (3.7); 1.117 (0.4); 1.082 (0.6); 0.000 (1.3)

### Example 30

¹H-NMR (400.0 MHz, d₆-DMSO): δ 10.167 (3.6); 8.824 (1.9); 8.821 (2.1); 8.812 (2.0); 8.809 (2.1); 8.209 (1.8); 8.189 (1.9); 7.952 (0.6); 7.738 (1.3); 7.726 (1.4); 7.719 (1.4); 7.707 (1.2); 7.335 (1.5); 7.312 (0.4); 7.306 (3.0); 7.301 (1.2); 7.294 (0.6); 7.288 (4.3); 7.284 (4.8); 7.271 (1.3); 7.266 (3.9); 7.260 (0.6); 7.199 (3.6); 7.184 (2.5); 7.164 (1.7); 7.072 (2.7); 7.064 (2.0); 7.053 (2.0); 7.017 (2.4); 6.997 (2.0); 6.977 (5.1); 6.956 (5.6); 6.938 (3.1); 6.920 (1.4); 4.817 (0.7); 4.803 (1.1); 4.790 (1.2); 4.776 (0.7); 4.156 (3.0); 4.151 (3.2); 4.141 (4.4); 4.124 (0.5); 3.329 (41.0); 2.889 (4.7); 2.730 (3.9); 2.524 (0.8); 2.510 (19.3); 2.506 (39.9); 2.501 (53.3); 2.497 (39.7); 2.492 (19.9); 2.328 (0.3); 2.075 (16.0); 2.032 (0.4); 1.383 (8.9); 1.368 (8.9); 0.000 (3.6)

### Example 31

¹H-NMR (400.0 MHz, d₆-DMSO): δ 10.166 (3.3); 8.824 (1.7); 8.821 (1.8); 8.812 (1.8); 8.809 (1.8); 8.208 (1.6); 8.189 (1.7); 7.952 (0.6); 7.739 (1.2); 7.727 (1.2); 7.719 (1.2); 7.707 (1.1); 7.334 (1.3); 7.199 (3.8); 7.179 (2.2); 7.159 (1.5); 7.088 (3.9); 7.068 (6.8); 7.050 (1.8); 7.006 (2.1); 6.985 (1.7); 6.870 (0.7); 6.863 (5.4); 6.842 (4.5); 4.792 (0.6); 4.778 (1.0); 4.766 (1.1); 4.751 (0.6); 4.136 (0.4); 4.110 (2.5); 4.103 (2.6); 4.095 (3.1); 4.093 (3.1); 4.077 (0.4); 3.334 (95.8); 2.889 (4.7); 2.730 (3.9); 2.524 (0.7); 2.510 (18.4); 2.506 (37.9); 2.501 (50.4); 2.497 (37.0); 2.493 (18.2); 2.328 (0.3); 2.224 (16.0); 2.073 (14.4); 1.369 (7.9); 1.354 (7.9); 0.000 (2.2)

### Example 32

¹H-NMR (400.0 MHz, d₆-DMSO): δ 10.160 (3.6); 8.822 (1.9); 8.819 (2.0); 8.810 (2.0); 8.807 (1.9); 8.206 (1.8); 8.187 (1.9); 7.952 (1.7); 7.735 (1.3); 7.723 (1.4); 7.716 (1.3); 7.704 (1.2); 7.332 (1.4); 7.203 (1.2); 7.196 (3.4); 7.183 (2.4); 7.162 (2.1); 7.136 (3.8); 7.117 (3.7); 7.073 (2.6); 7.061 (1.9); 7.054 (2.1); 7.039 (2.4); 7.019 (1.8); 6.964 (2.5); 6.944 (2.2); 6.853 (1.6); 6.835 (2.8); 6.817 (1.3); 4.853 (0.6); 4.841 (1.2); 4.826 (1.2); 4.814 (0.7); 4.144 (5.0); 4.132 (4.2); 3.338 (159.7); 2.890 (12.2); 2.731 (10.6); 2.671 (0.4); 2.524 (0.8); 2.511 (21.8); 2.506 (43.7); 2.502 (57.6); 2.497 (42.4); 2.493 (21.1); 2.329 (0.4); 2.136 (0.4); 2.114 (16.0); 2.085 (15.9); 1.411 (8.5); 1.396 (8.4); 0.000 (1.1)

### Example 33

¹H-NMR (400.0 MHz, d₆-DMSO): δ 10.169 (3.3); 8.824 (1.7); 8.821 (1.8); 8.812 (1.8); 8.810 (1.8); 8.209 (1.6); 8.190 (1.7); 7.952 (0.4); 7.739 (1.2); 7.727 (1.2); 7.720 (1.2); 7.707 (1.1); 7.334 (1.3); 7.199 (3.1); 7.184 (2.2); 7.172 (1.8); 7.164 (1.6); 7.152 (3.2); 7.133 (2.0); 7.071 (2.4); 7.064 (1.8); 7.052 (1.7); 7.013 (2.1); 6.992 (1.7); 6.791 (2.6); 6.764 (3.4); 6.760 (3.1); 6.744 (3.4); 4.804 (0.6); 4.790 (1.0); 4.777 (1.1); 4.763 (0.6); 4.131 (2.5); 4.125 (2.7); 4.116 (3.5); 4.099 (0.4); 3.338 (126.9); 2.889 (2.9); 2.730 (2.5); 2.524 (0.7); 2.510 (17.9); 2.506 (36.1); 2.502 (47.6); 2.497 (35.1); 2.493 (17.5); 2.265 (16.0); 2.075 (13.9); 1.376 (7.5); 1.361 (7.5); 0.000 (1.4)

### Example 34

¹H-NMR (400.0 MHz, d₆-DMSO): δ 10.167 (3.9); 8.822 (2.1); 8.812 (2.1); 8.810 (2.1); 8.209 (1.8); 8.189 (2.0); 7.953 (0.7); 7.739 (1.4); 7.727 (1.4); 7.720 (1.4); 7.708 (1.3); 7.334 (1.5); 7.322 (2.1); 7.301 (4.3); 7.281 (2.7); 7.199 (3.5); 7.185 (2.6); 7.165 (1.7); 7.083 (2.3); 7.077 (5.6); 7.073 (5.0); 7.063 (2.1); 7.055 (2.2); 7.012 (4.6); 6.992 (3.8); 6.965 (1.9); 6.959 (1.8); 6.944 (1.7); 6.938 (1.6); 4.813 (0.7); 4.800 (1.3); 4.786 (1.4); 4.772 (0.7); 4.201 (5.1); 4.189 (5.1); 3.336 (110.6); 2.890 (4.4); 2.731 (3.9); 2.671 (0.4); 2.506 (43.4); 2.502 (57.0); 2.498 (42.2); 2.329 (0.4); 2.063 (16.0); 1.371 (8.8); 1.356 (8.7); 0.000 (2.5)

### Example 35

¹H-NMR (400.0 MHz, d₆-DMSO): δ 10.159 (3.6); 8.822 (1.8); 8.819 (2.0); 8.811 (1.9); 8.808 (1.9); 8.206 (1.7); 8.187 (1.8); 7.952 (1.8); 7.736 (1.3); 7.724 (1.3); 7.717 (1.3); 7.704 (1.2); 7.432 (2.4); 7.428 (2.7); 7.412 (2.7); 7.408 (2.9); 7.331 (1.4); 7.319 (1.0); 7.315 (1.0); 7.298 (2.1); 7.294 (1.9); 7.279 (1.8); 7.275 (1.7); 7.217 (2.6); 7.214 (2.9); 7.205 (1.2); 7.196 (5.0); 7.185 (2.5); 7.165 (1.7); 7.077 (2.6); 7.058 (4.6); 7.036 (1.8); 6.982 (1.5); 6.978 (1.6); 6.962 (2.4); 6.959 (2.4); 6.944 (1.3); 6.940 (1.3); 4.849 (0.6); 4.837 (1.1); 4.823 (1.2); 4.809 (0.7); 4.252 (3.9); 4.240 (4.7); 3.345 (217.9); 3.308 (0.4); 2.890 (13.2); 2.731 (11.3); 2.672 (0.4); 2.525 (1.0); 2.511 (21.8); 2.507 (44.8); 2.503 (59.6); 2.498 (44.0); 2.494 (21.7); 2.329 (0.4); 2.073 (16.0); 1.411 (8.6); 1.395 (8.5); 0.000 (0.5)

### Example 36

¹H-NMR (400.0 MHz, d₆-DMSO): δ 10.212 (3.5); 8.828 (1.8); 8.825 (2.0); 8.817 (1.9); 8.813 (1.9); 8.220 (1.7); 8.200 (1.8); 7.952 (0.6); 7.745 (1.3); 7.732 (1.4); 7.725 (1.3); 7.713 (1.2); 7.549 (2.2); 7.535 (2.7); 7.528 (3.0); 7.514 (2.6); 7.339 (1.5); 7.270 (0.4); 7.262 (3.4); 7.257 (1.2); 7.245 (1.3); 7.240 (5.9); 7.234 (1.4); 7.223 (1.1); 7.218 (3.1); 7.210 (1.5); 7.204 (3.5); 7.190 (2.5); 7.170 (1.6); 7.072 (2.9); 7.053 (1.9); 6.991 (2.3); 6.971 (1.9); 5.133 (8.1); 3.350 (196.6); 3.304 (0.3); 2.890 (4.4); 2.731 (3.7); 2.525 (0.8); 2.520 (1.2); 2.512 (18.2); 2.507 (37.6); 2.503 (50.0); 2.498 (36.8); 2.494 (18.1); 2.132 (16.0); 1.202 (0.4); 1.187 (0.4)

### Example 37

¹H-NMR (400.0 MHz, d₆-DMSO): δ 10.218 (3.3); 8.829 (2.1); 8.826 (2.1); 8.818 (2.2); 8.814 (2.0); 8.225 (1.9); 8.205 (2.0); 7.952 (0.7); 7.746 (1.4); 7.733 (1.6); 7.726 (1.5); 7.714 (1.4); 7.653 (1.4); 7.642 (1.6); 7.636 (1.0); 7.630 (1.7); 7.548 (1.7); 7.542 (1.0); 7.536 (1.7); 7.533 (1.5); 7.525 (2.4); 7.516 (0.5); 7.511 (0.6); 7.507 (0.4); 7.494 (0.4); 7.488 (0.6); 7.435 (0.6); 7.430 (0.5); 7.422 (4.4); 7.416 (2.5); 7.410 (3.7); 7.406 (2.5); 7.399 (4.0); 7.390 (0.8); 7.343 (1.7); 7.316 (0.5); 7.303 (0.6); 7.233 (0.9); 7.213 (2.7); 7.208 (4.1); 7.193 (1.6); 7.099 (2.5); 7.080 (1.9); 7.072 (2.0); 7.016 (2.2); 6.996 (1.8); 5.199 (8.9); 5.006 (0.5); 3.329 (48.2); 2.890 (4.5); 2.730 (3.9); 2.671 (0.4); 2.524 (0.9); 2.510 (23.9); 2.506 (49.3); 2.502 (65.7); 2.497 (48.8); 2.493 (24.4); 2.328 (0.4); 2.181 (0.6); 2.167 (0.4); 2.139 (16.0); 1.232 (1.7); 1.216 (1.9); 1.147 (0.6); 1.134 (0.6); 1.123 (0.6); 0.000 (4.5)

### Example 38

¹H-NMR (400.0 MHz, d₆-DMSO): δ 10.227 (3.6); 8.828 (2.1); 8.819 (2.0); 8.816 (2.1); 8.227 (1.7); 8.208 (1.9); 7.953 (1.0); 7.845 (3.2); 7.815 (1.5); 7.796 (2.0); 7.748 (1.4); 7.736 (1.5); 7.728 (2.4); 7.716 (1.6); 7.707 (2.7); 7.684 (2.3); 7.665 (2.4); 7.646 (0.9); 7.343 (1.5); 7.225 (1.0); 7.207 (4.8); 7.185 (1.7); 7.093 (2.6); 7.073 (3.5); 7.000 (2.4); 6.980 (2.0); 5.267 (8.5); 3.331 (64.9); 2.890 (6.6); 2.731 (5.7); 2.671 (0.4); 2.524 (0.9); 2.511 (24.1); 2.506 (49.8); 2.502 (66.4); 2.497 (49.5); 2.493 (24.9); 2.333 (0.3); 2.329 (0.4); 2.324 (0.3); 2.180 (0.7); 2.161 (16.0); 2.139 (0.4); 2.122 (0.4); 0.000 (4.4)

### Example 39

¹H-NMR (400.0 MHz, d₆-DMSO): δ 10.222 (3.6); 8.826 (2.1); 8.817 (2.2); 8.226 (1.8); 8.206 (2.0); 7.953 (0.7); 7.832 (2.3); 7.821 (1.6); 7.813 (2.8); 7.803 (2.7); 7.773 (1.6); 7.755 (2.5); 7.746 (1.7); 7.734 (2.4); 7.727 (1.5); 7.714 (1.3); 7.625 (1.3); 7.606 (2.0); 7.587 (0.9); 7.343 (1.5); 7.233 (1.0); 7.213 (2.7); 7.207 (3.7); 7.193 (1.6); 7.107 (2.6); 7.087 (1.9); 7.072 (1.7); 6.976 (2.3); 6.955 (2.0); 5.273 (6.5); 3.330 (65.7); 2.890 (4.1); 2.731 (3.7); 2.671 (0.4); 2.506 (48.3); 2.502 (63.4); 2.497 (47.0); 2.328 (0.4); 2.180 (0.4); 2.122 (16.0); 1.224 (0.8); 1.208 (0.8); 1.188 (0.4); 1.127 (0.4); 0.000 (3.7)

### Example 40

¹H-NMR (400.0 MHz, d₆-DMSO): δ 10.220 (3.1); 9.006 (0.4); 9.002 (0.5); 8.994 (0.5); 8.990 (0.5); 8.866 (0.4); 8.855 (0.5); 8.827 (1.8); 8.818 (1.8); 8.816 (1.8); 8.692 (0.4); 8.674 (0.4); 8.226 (1.6); 8.207 (1.7); 7.952 (2.4); 7.747 (1.2); 7.735 (1.3); 7.728 (1.3); 7.716 (1.1); 7.495 (0.6); 7.490 (0.7); 7.469 (1.4); 7.454 (1.6); 7.450 (1.3); 7.434 (1.2); 7.416 (1.2); 7.361 (0.3); 7.336 (2.3); 7.317 (2.8); 7.290 (1.3); 7.208 (3.3); 7.190 (2.9); 7.170 (2.8); 7.148 (0.7); 7.143 (0.7); 7.081 (2.4); 7.072 (1.7); 7.062 (1.8); 6.974 (2.0); 6.953 (1.7); 5.183 (7.6); 3.333 (108.5); 2.890 (16.0); 2.730 (14.0); 2.671 (0.4); 2.667 (0.3); 2.506 (50.0); 2.502 (66.4); 2.497 (51.1); 2.329 (0.4); 2.324 (0.3); 2.159 (13.6); 2.138 (2.8); 1.202 (1.9); 1.187 (1.7); 1.110 (0.4); 1.069 (0.6); 0.000 (3.2)

### Example 41

¹H-NMR (400.0 MHz, d₆-DMSO): δ 10.209 (2.7); 9.006 (0.7); 9.003 (0.8); 8.994 (0.7); 8.991 (0.8); 8.867 (0.7); 8.855 (0.7); 8.824 (1.6); 8.815 (1.5); 8.693 (0.7); 8.673 (0.7); 8.263 (0.3); 8.220 (1.4); 8.201 (1.5); 7.953 (2.5); 7.870 (0.5); 7.858 (0.5); 7.850 (0.5); 7.839 (0.5); 7.767 (0.3); 7.743 (1.1); 7.731 (1.1); 7.723 (1.1); 7.711 (0.9); 7.630 (0.4); 7.611 (0.7); 7.592 (1.4); 7.573 (0.8); 7.495 (0.9); 7.459 (0.4); 7.455 (0.5); 7.438 (1.1); 7.417 (2.6); 7.408 (0.9); 7.361 (0.5); 7.338 (1.1); 7.294 (1.3); 7.276 (2.0); 7.270 (1.6); 7.260 (2.3); 7.247 (1.0); 7.242 (1.2); 7.229 (0.8); 7.209 (2.2); 7.203 (2.9); 7.189 (1.5); 7.091 (2.1); 7.069 (2.0); 7.049 (2.0); 7.028 (1.5); 5.187 (6.4); 4.819 (0.3); 4.803 (0.3); 4.787 (0.4); 4.772 (0.4); 3.335 (137.4); 2.890 (16.0); 2.731 (14.1); 2.671 (0.4); 2.506 (51.4); 2.502 (67.4); 2.498 (51.1); 2.329 (0.4); 2.138 (4.4); 2.106 (12.2); 1.936 (0.4); 1.202 (3.1); 1.187 (2.8); 1.074 (1.0); 0.000 (2.7)

### Example 42

¹H-NMR (400.0 MHz, d₆-DMSO): δ 10.231 (3.9); 8.829 (2.5); 8.818 (2.2); 8.229 (2.0); 8.209 (2.1); 7.953 (0.3); 7.749 (1.5); 7.737 (1.7); 7.730 (1.6); 7.718 (1.3); 7.596 (2.4); 7.591 (4.3); 7.587 (2.9); 7.563 (1.2); 7.535 (7.6); 7.532 (7.4); 7.345 (1.6); 7.312 (0.8); 7.221 (1.2); 7.210 (3.7); 7.201 (2.8); 7.181 (1.7); 7.099 (2.8); 7.075 (2.5); 6.952 (2.4); 6.932 (2.1); 5.188 (8.8); 4.902 (0.5); 3.335 (74.0); 2.890 (2.0); 2.731 (1.8); 2.507 (32.7); 2.502 (43.4); 2.498 (33.9); 2.180 (1.5); 2.162 (16.0); 1.241 (1.4); 1.225 (1.5); 1.164 (0.5); 0.000 (2.1)

### Example 43

¹H-NMR (400.0 MHz, d₆-DMSO): δ 10.353 (0.8); 10.233 (4.1); 9.004 (0.5); 8.995 (0.6); 8.974 (1.8); 8.962 (1.9); 8.828 (2.8); 8.819 (2.7); 8.620 (0.4); 8.600 (0.5); 8.493 (1.5); 8.473 (1.7); 8.256 (0.5); 8.228 (2.2); 8.208 (2.3); 7.953 (1.3); 7.848 (0.3); 7.829 (0.4); 7.817 (0.4); 7.805 (1.2); 7.793 (1.2); 7.786 (1.2); 7.773 (1.1); 7.748 (1.8); 7.736 (2.0); 7.729 (2.0); 7.717 (1.5); 7.697 (3.5); 7.692 (3.7); 7.597 (2.8); 7.575 (4.4); 7.508 (2.7); 7.502 (2.7); 7.487 (1.8); 7.481 (1.7); 7.453 (0.7); 7.439 (1.1); 7.414 (0.6); 7.393 (0.8); 7.370 (0.7); 7.348 (2.5); 7.327 (3.6); 7.304 (2.4); 7.266 (1.7); 7.260 (1.9); 7.245 (2.1); 7.238 (1.7); 7.226 (3.1); 7.207 (5.1); 7.169 (1.3); 7.140 (0.6); 7.116 (2.9); 7.096 (2.3); 7.072 (1.7); 7.054 (2.7); 7.049 (2.6); 7.020 (2.7); 7.000 (2.3); 6.829 (1.9); 6.809 (2.2); 6.610 (2.3); 6.589 (2.0); 6.547 (2.4); 5.188 (9.5); 5.130 (4.2); 4.049 (1.5); 3.831 (4.3); 3.354 (1010.6); 2.891 (7.0); 2.750 (0.5); 2.732 (6.4); 2.673 (1.4); 2.504 (210.9); 2.331 (1.4); 2.173 (0.5); 2.142 (16.0); 2.087 (2.9); 2.048 (0.4); 2.011 (0.3); 1.855 (11.5); 1.259 (0.6); 1.241 (1.1); 1.224 (0.7); 0.000 (4.2)

### Example 44

¹H-NMR (400.0 MHz, d₆-DMSO): δ 10.223 (3.9); 8.828 (2.3); 8.818 (2.3); 8.226 (1.9); 8.206 (2.1); 7.953 (0.8); 7.746 (4.8); 7.742 (4.6); 7.729 (1.8); 7.717 (1.4); 7.696 (3.6); 7.685 (0.5); 7.675 (4.4); 7.495 (2.2); 7.491 (2.2); 7.474 (1.9); 7.470 (1.9); 7.343 (1.5); 7.207 (3.4); 7.195 (2.6); 7.175 (1.7); 7.090 (2.8); 7.071 (3.4); 6.962 (2.5); 6.942 (2.1); 5.175 (9.1); 3.330 (42.7); 2.890 (4.5); 2.731 (4.1); 2.506 (39.6); 2.502 (50.3); 2.498 (39.0); 2.329 (0.3); 2.152 (16.0); 1.239 (0.5); 1.224 (0.5); 0.000 (2.8)

### Example 45

¹H-NMR (400.0 MHz, d₆-DMSO): δ 10.226 (3.6); 8.831 (1.9); 8.828 (2.0); 8.819 (2.0); 8.816 (2.0); 8.227 (1.8); 8.207 (1.9); 7.953 (0.4); 7.748 (1.3); 7.736 (1.4); 7.728 (1.3); 7.716 (1.2); 7.681 (2.0); 7.677 (2.3); 7.661 (2.4); 7.657 (2.8); 7.631 (1.8); 7.614 (2.3); 7.467 (2.7); 7.447 (4.3); 7.427 (1.9); 7.344 (1.5); 7.236 (1.0); 7.216 (2.6); 7.208 (3.7); 7.196 (1.7); 7.111 (2.6); 7.091 (1.9); 7.073 (1.7); 7.008 (2.3); 6.988 (1.9); 5.241 (8.8); 3.329 (36.8); 2.890 (2.7); 2.731 (2.3); 2.524 (0.9); 2.511 (18.6); 2.506 (37.8); 2.502 (50.1); 2.497 (37.1); 2.493 (18.3); 2.148 (16.0); 1.227 (0.5); 1.211 (0.5); 0.000 (4.0)

### Example 46

¹H-NMR (400.0 MHz, d₆-DMSO): δ 10.221 (3.7); 8.827 (2.0); 8.818 (2.0); 8.815 (2.0); 8.224 (1.8); 8.205 (1.9); 7.746 (1.3); 7.734 (1.4); 7.726 (1.4); 7.718 (4.7); 7.713 (5.4); 7.670 (2.7); 7.649 (3.6); 7.523 (2.7); 7.518 (2.6); 7.503 (2.1); 7.497 (2.0); 7.342 (1.5); 7.234 (1.0); 7.214 (2.6); 7.207 (3.6); 7.194 (1.7); 7.106 (2.7); 7.087 (2.0); 7.071 (1.7); 7.007 (2.3); 6.986 (2.0); 5.184 (8.9); 3.331 (33.7); 2.890 (1.2); 2.731 (1.0); 2.524 (0.5); 2.511 (13.1); 2.506 (26.8); 2.502 (35.7); 2.497 (26.6); 2.493 (13.4); 2.133 (16.0); 0.000 (2.7)

### Example 47

¹H-NMR (400.0 MHz, d₆-DMSO): δ 10.169 (3.7); 8.824 (2.0); 8.815 (2.0); 8.812 (2.0); 8.212 (1.8); 8.192 (1.9); 7.952 (2.3); 7.743 (1.3); 7.731 (1.4); 7.724 (1.4); 7.712 (1.2); 7.463 (2.3); 7.442 (7.8); 7.424 (9.2); 7.408 (1.2); 7.403 (2.7); 7.385 (2.3); 7.334 (1.4); 7.198 (3.1); 7.072 (1.0); 7.063 (1.7); 7.052 (2.4); 7.032 (1.8); 6.992 (2.8); 6.972 (1.6); 6.758 (2.2); 6.738 (2.0); 5.577 (0.5); 5.561 (1.8); 5.545 (1.8); 5.529 (0.6); 4.777 (0.4); 3.330 (72.5); 2.890 (15.7); 2.730 (13.7); 2.670 (0.4); 2.524 (0.9); 2.510 (21.9); 2.506 (44.7); 2.502 (59.6); 2.497 (44.7); 2.333 (0.3); 2.328 (0.4); 2.175 (16.0); 1.974 (1.4); 1.569 (7.9); 1.553 (7.9); 1.502 (0.7); 1.486 (0.7); 0.000 (3.9)

### Example 48

¹H-NMR (400.0 MHz, d₆-DMSO): δ 10.179 (3.2); 8.828 (1.6); 8.825 (1.7); 8.816 (1.7); 8.813 (1.7); 8.216 (1.5); 8.197 (1.6); 7.953 (2.2); 7.745 (1.1); 7.733 (1.2); 7.725 (1.2); 7.713 (1.1); 7.701 (3.1); 7.696 (3.2); 7.639 (3.4); 7.618 (4.4); 7.607 (0.4); 7.586 (0.4); 7.435 (1.8); 7.430 (1.8); 7.414 (1.5); 7.409 (1.5); 7.336 (1.2); 7.201 (2.8); 7.095 (0.8); 7.075 (2.1); 7.065 (1.5); 7.055 (1.6); 7.015 (2.4); 6.996 (1.4); 6.775 (1.9); 6.755 (1.7); 5.600 (0.5); 5.585 (1.5); 5.569 (1.6); 5.553 (0.5); 4.806 (0.4); 3.332 (67.5); 2.890 (16.0); 2.731 (13.6); 2.671 (0.3); 2.525 (0.9); 2.511 (19.6); 2.507 (40.3); 2.502 (53.5); 2.498 (39.3); 2.493 (19.4); 2.329 (0.4); 2.182 (13.8); 1.979 (1.4); 1.579 (6.7); 1.563 (6.7); 1.513 (0.7); 1.497 (0.7); 0.000 (3.4)

### Example 49

¹H-NMR (400.0 MHz, d₆-DMSO): δ 10.165 (3.6); 8.823 (2.1); 8.814 (2.0); 8.210 (1.8); 8.190 (2.0); 7.952 (2.0); 7.742 (1.3); 7.730 (1.4); 7.723 (1.4); 7.711 (1.2); 7.488 (2.2); 7.474 (2.8); 7.467 (3.0); 7.453 (2.6); 7.332 (1.4); 7.197 (4.3); 7.178 (5.3); 7.156 (2.5); 7.074 (1.0); 7.061 (1.8); 7.054 (2.5); 7.034 (1.8); 6.987 (2.9); 6.967 (1.7); 6.782 (2.3); 6.762 (2.0); 5.577 (0.6); 5.562 (1.8); 5.546 (1.8); 5.530 (0.6); 3.342 (145.0); 2.890 (12.8); 2.731 (11.4); 2.671 (0.3); 2.507 (39.7); 2.502 (51.1); 2.498 (37.9); 2.329 (0.4); 2.171 (16.0); 1.570 (8.0); 1.555 (8.0); 0.000 (1.0)

### Example 50

¹H-NMR (400.0 MHz, d₆-DMSO): δ 10.197 (3.0); 8.829 (1.6); 8.827 (1.8); 8.818 (1.7); 8.815 (1.7); 8.218 (1.5); 8.199 (1.6); 7.952 (2.2); 7.747 (1.1); 7.734 (1.2); 7.727 (1.2); 7.715 (1.0); 7.500 (1.7); 7.495 (1.7); 7.490 (1.5); 7.485 (1.7); 7.481 (2.5); 7.477 (2.4); 7.472 (1.8); 7.467 (1.9); 7.372 (0.6); 7.368 (0.8); 7.354 (2.0); 7.350 (1.9); 7.335 (4.1); 7.330 (2.6); 7.316 (1.7); 7.311 (1.7); 7.297 (0.7); 7.292 (0.6); 7.200 (2.7); 7.078 (0.8); 7.065 (1.6); 7.058 (2.2); 7.038 (1.6); 7.001 (2.4); 6.982 (1.3); 6.547 (0.4); 6.535 (1.9); 6.516 (1.7); 5.741 (0.5); 5.725 (1.7); 5.709 (1.7); 5.693 (0.5); 3.351 (239.6); 3.310 (0.6); 2.890 (16.0); 2.731 (13.5); 2.672 (0.4); 2.525 (0.9); 2.512 (20.4); 2.507 (41.7); 2.503 (55.1); 2.498 (40.4); 2.494 (19.8); 2.329 (0.4); 2.217 (14.0); 2.017 (0.4); 1.613 (7.3); 1.597 (7.3); 0.000 (0.6)

### Example 51

¹H-NMR (400.0 MHz, d₆-DMSO): δ 10.175 (3.7); 8.825 (2.1); 8.813 (2.1); 8.216 (1.8); 8.197 (2.0); 7.952 (2.1); 7.744 (1.3); 7.732 (1.4); 7.724 (1.4); 7.712 (1.2); 7.485 (3.3); 7.400 (4.4); 7.388 (5.9); 7.367 (0.5); 7.343 (2.1); 7.337 (3.0); 7.331 (1.9); 7.326 (1.8); 7.320 (0.9); 7.315 (0.8); 7.305 (0.3); 7.201 (3.0); 7.085 (0.9); 7.066 (3.9); 7.045 (1.8); 7.004 (2.9); 6.984 (1.7); 6.775 (2.3); 6.754 (2.1); 5.585 (0.6); 5.570 (1.9); 5.554 (1.9); 5.538 (0.6); 4.792 (0.4); 3.329 (53.0); 2.889 (13.3); 2.730 (12.1); 2.670 (0.4); 2.506 (45.0); 2.501 (59.3); 2.497 (45.5); 2.328 (0.4); 2.186 (16.0); 1.984 (1.5); 1.580 (7.9); 1.565 (7.9); 1.514 (0.7); 1.498 (0.7); 0.000 (2.9)

### Example 52

¹H-NMR (400.0 MHz, d₆-DMSO): δ 10.146 (3.4); 8.820 (1.8); 8.817 (2.0); 8.808 (2.0); 8.805 (2.0); 8.201 (1.7); 8.182 (1.8); 7.952 (1.7); 7.733 (1.3); 7.721 (1.4); 7.714 (1.3); 7.702 (1.2); 7.431 (0.4); 7.415 (1.0); 7.410 (0.9); 7.394 (1.8); 7.377 (1.0); 7.373 (1.1); 7.356 (0.5); 7.323 (1.4); 7.188 (3.1); 7.123 (1.3); 7.115 (2.9); 7.103 (2.9); 7.094 (5.1); 7.083 (2.3); 7.072 (2.4); 7.052 (1.6); 7.025 (2.7); 7.005 (1.7); 6.815 (2.2); 6.795 (1.9); 5.835 (0.5); 5.819 (1.7); 5.803 (1.7); 5.787 (0.5); 3.330 (54.5); 2.890 (12.2); 2.730 (10.5); 2.670 (0.4); 2.524 (0.9); 2.510 (20.8); 2.506 (42.2); 2.501 (55.8); 2.497 (41.1); 2.493 (20.3); 2.328 (0.3); 2.120 (16.0); 1.751 (7.6); 1.735 (7.6); 1.237 (0.4); 0.000 (3.5)

### Example 53

¹H-NMR (400.0 MHz, d₆-DMSO): δ 10.178 (3.7); 8.828 (1.8); 8.825 (2.0); 8.816 (2.0); 8.813 (2.0); 8.216 (1.7); 8.196 (1.9); 7.953 (1.7); 7.800 (3.1); 7.755 (1.5); 7.733 (2.6); 7.725 (1.6); 7.713 (1.3); 7.658 (1.0); 7.638 (2.8); 7.626 (2.5); 7.607 (2.4); 7.589 (0.8); 7.334 (1.4); 7.199 (3.2); 7.092 (0.9); 7.072 (2.5); 7.064 (1.8); 7.052 (1.8); 7.011 (2.8); 6.991 (1.6); 6.806 (2.2); 6.786 (1.9); 5.710 (0.5); 5.694 (1.8); 5.678 (1.8); 5.663 (0.6); 4.802 (0.4); 3.329 (55.0); 2.890 (12.4); 2.731 (10.6); 2.671 (0.4); 2.524 (1.0); 2.511 (23.9); 2.506 (49.1); 2.502 (65.2); 2.497 (48.0); 2.493 (23.7); 2.328 (0.4); 2.194 (16.0); 1.992 (1.7); 1.606 (7.9); 1.590 (7.9); 1.540 (0.9); 1.524 (0.8); 0.000 (4.6)

### Example 54

¹H-NMR (400.0 MHz, d₆-DMSO): δ 10.221 (3.7); 8.828 (2.1); 8.819 (2.0); 8.816 (2.0); 8.227 (1.8); 8.207 (1.9); 7.953 (0.6); 7.747 (1.4); 7.735 (1.4); 7.728 (1.4); 7.716 (1.3); 7.680 (3.5); 7.550 (1.6); 7.530 (2.1); 7.504 (1.7); 7.484 (2.4); 7.402 (2.8); 7.382 (4.0); 7.363 (1.6); 7.346 (1.5); 7.210 (4.0); 7.192 (2.5); 7.172 (1.7); 7.085 (2.7); 7.075 (1.9); 7.066 (2.0); 6.969 (2.4); 6.949 (2.0); 5.172 (8.6); 3.332 (22.9); 2.889 (4.2); 2.730 (3.7); 2.524 (0.4); 2.510 (9.5); 2.506 (19.7); 2.501 (26.3); 2.497 (19.6); 2.493 (9.9); 2.155 (16.0); 0.000 (1.9)

### Example 55

¹H-NMR (400.0 MHz, d₆-DMSO): □□ 10.191 (3.6); 8.826 (2.0); 8.817 (2.0); 8.218 (1.8); 8.198 (1.9); 7.953 (0.7); 7.775 (2.9); 7.755 (4.2); 7.734 (1.5); 7.725 (2.5); 7.715 (1.5); 7.705 (2.2); 7.686 (1.0); 7.535 (1.3); 7.516 (2.1); 7.497 (0.9); 7.334 (1.3); 7.199 (3.0); 7.064 (2.0); 7.046 (2.3); 7.026 (2.1); 7.012 (3.0); 6.993 (1.2); 6.597 (2.0); 6.577 (1.8); 5.714 (0.4); 5.699 (1.3); 5.683 (1.3); 5.669 (0.5); 3.331 (78.0); 2.890 (4.5); 2.731 (4.0); 2.671 (0.4); 2.506 (53.2); 2.502 (68.1); 2.498 (50.1); 2.329 (0.4); 2.222 (16.0); 1.639 (7.4); 1.624 (7.4); 0.000 (4.0)

### Example 56

¹H-NMR (400.0 MHz, d₆-DMSO): δ 10.208 (3.8); 8.828 (2.1); 8.819 (2.1); 8.817 (2.1); 8.228 (1.8); 8.209 (2.0); 7.953 (1.0); 7.748 (1.3); 7.736 (1.5); 7.728 (1.4); 7.716 (1.2); 7.562 (3.3); 7.540 (4.5); 7.472 (3.1); 7.465 (3.8); 7.425 (2.9); 7.419 (2.3); 7.404 (2.1); 7.397 (1.8); 7.343 (1.4); 7.208 (3.0); 7.109 (0.9); 7.089 (2.4); 7.071 (2.8); 7.044 (3.0); 7.025 (1.4); 6.556 (2.2); 6.536 (2.0); 5.708 (0.6); 5.693 (2.0); 5.677 (2.0); 5.661 (0.6); 3.335 (98.4); 2.890 (6.7); 2.731 (5.9); 2.506 (36.2); 2.502 (46.9); 2.498 (35.6); 2.221 (16.0); 1.617 (8.2); 1.602 (8.2); 0.000 (2.2)

### Example 57

¹H-NMR (400.0 MHz, d₆-DMSO): δ 10.180 (3.5); 8.827 (1.8); 8.824 (2.0); 8.815 (1.9); 8.812 (1.9); 8.213 (1.7); 8.193 (1.8); 7.952 (1.7); 7.743 (1.3); 7.731 (1.3); 7.723 (1.3); 7.711 (1.2); 7.483 (0.9); 7.464 (1.8); 7.445 (1.0); 7.368 (0.4); 7.364 (0.4); 7.354 (0.5); 7.350 (1.1); 7.333 (2.3); 7.325 (1.0); 7.316 (0.8); 7.311 (0.7); 7.242 (1.5); 7.217 (3.7); 7.198 (6.5); 7.181 (1.1); 7.093 (0.9); 7.073 (2.4); 7.062 (1.7); 7.053 (1.8); 7.008 (2.8); 6.988 (1.6); 6.739 (2.2); 6.719 (1.9); 5.741 (0.5); 5.725 (1.8); 5.709 (1.8); 5.693 (0.6); 3.390 (0.8); 3.353 (250.4); 2.890 (12.5); 2.731 (10.6); 2.672 (0.4); 2.525 (0.9); 2.512 (20.5); 2.507 (41.5); 2.503 (54.8); 2.498 (40.3); 2.494 (19.9); 2.330 (0.4); 2.182 (16.0); 1.630 (8.0); 1.614 (8.0); 0.000 (0.3)

### Example 58

¹H-NMR (400.0 MHz, d₆-DMSO): δ 10.162 (3.9); 8.819 (2.2); 8.808 (2.2); 8.203 (1.9); 8.183 (2.1); 7.952 (1.0); 7.735 (1.4); 7.723 (1.6); 7.716 (1.5); 7.704 (1.5); 7.693 (4.0); 7.673 (5.9); 7.595 (5.1); 7.575 (3.6); 7.324 (1.5); 7.188 (4.2); 7.167 (2.6); 7.147 (1.7); 7.053 (1.6); 7.034 (2.8); 7.014 (2.2); 6.914 (2.5); 6.894 (2.2); 4.264 (2.2); 4.249 (4.8); 4.233 (2.4); 3.330 (71.9); 3.190 (2.2); 3.175 (4.2); 3.160 (2.1); 2.889 (5.7); 2.730 (5.3); 2.670 (0.4); 2.505 (48.0); 2.501 (62.3); 2.498 (49.7); 2.328 (0.4); 2.002 (16.0); 0.000 (2.6)

### Example 59

¹H-NMR (400.0 MHz, d₆-DMSO): δ 10.176 (3.1); 8.822 (2.2); 8.811 (2.2); 8.207 (1.9); 8.188 (2.1); 7.953 (2.8); 7.739 (3.2); 7.721 (4.1); 7.706 (1.7); 7.679 (0.7); 7.665 (4.6); 7.650 (2.3); 7.624 (1.1); 7.489 (1.0); 7.469 (1.6); 7.455 (1.1); 7.326 (1.3); 7.243 (0.4); 7.228 (0.7); 7.190 (3.2); 7.167 (2.1); 7.147 (1.4); 7.055 (1.5); 7.038 (2.3); 7.018 (1.9); 6.918 (2.0); 6.898 (1.8); 4.265 (1.8); 4.249 (3.7); 4.233 (2.0); 4.049 (0.4); 3.329 (94.6); 3.283 (1.9); 3.267 (3.5); 3.251 (2.2); 2.890 (16.0); 2.731 (14.8); 2.671 (0.6); 2.502 (93.9); 2.366 (0.4); 2.329 (0.7); 2.059 (1.1); 2.020 (12.6); 2.003 (2.3); 1.320 (1.1); 1.305 (1.0); 1.257 (0.6); 1.232 (2.4); 1.217 (2.7); 1.180 (1.4); 1.165 (1.2); 1.061 (0.7); 0.000 (4.4)

### Example 60

¹H-NMR (400.0 MHz, d₆-DMSO): δ 10.160 (3.7); 8.819 (2.1); 8.810 (2.1); 8.807 (2.1); 8.204 (1.8); 8.185 (2.0); 7.953 (1.7); 7.737 (4.4); 7.723 (1.6); 7.716 (1.5); 7.704 (1.3); 7.680 (1.6); 7.662 (2.2); 7.606 (1.0); 7.587 (3.1); 7.575 (2.8); 7.556 (2.4); 7.537 (0.8); 7.322 (1.5); 7.186 (4.1); 7.164 (2.5); 7.144 (1.7); 7.051 (1.7); 7.034 (2.7); 7.014 (2.1); 6.911 (2.4); 6.891 (2.1); 4.249 (2.2); 4.233 (4.7); 4.218 (2.4); 3.332 (79.7); 3.198 (2.2); 3.182 (4.5); 3.167 (2.2); 2.890 (11.3); 2.731 (9.7); 2.671 (0.4); 2.524 (0.9); 2.506 (46.3); 2.502 (60.9); 2.498 (45.8); 2.329 (0.4); 2.006 (16.0); 0.000 (3.1)

### Example 61

¹H-NMR (400.0 MHz, d₆-DMSO): δ 10.169 (3.5); 8.820 (2.0); 8.811 (2.0); 8.808 (2.0); 8.208 (1.8); 8.189 (2.0); 7.952 (1.0); 7.737 (1.3); 7.725 (1.4); 7.717 (1.4); 7.705 (1.2); 7.384 (0.9); 7.369 (1.1); 7.364 (1.9); 7.349 (2.1); 7.345 (1.6); 7.328 (2.7); 7.218 (1.5); 7.214 (1.4); 7.198 (3.2); 7.193 (5.2); 7.187 (3.3); 7.168 (2.6); 7.148 (1.6); 7.077 (0.9); 7.071 (0.9); 7.057 (2.8); 7.050 (1.6); 7.034 (3.3); 7.015 (2.1); 6.913 (2.3); 6.893 (2.0); 4.233 (2.2); 4.217 (4.9); 4.201 (2.4); 3.333 (73.7); 3.111 (2.3); 3.095 (4.6); 3.079 (2.2); 2.889 (6.9); 2.730 (6.0); 2.524 (0.7); 2.510 (17.7); 2.506 (35.8); 2.502 (47.5); 2.497 (35.2); 2.493 (17.7); 2.023 (16.0); 0.000 (2.5)

### Example 62

¹H-NMR (400.0 MHz, d₆-DMSO): δ 10.163 (3.5); 8.818 (2.1); 8.806 (2.1); 8.203 (1.8); 8.183 (2.0); 7.952 (1.4); 7.735 (1.3); 7.722 (1.4); 7.716 (1.4); 7.703 (1.3); 7.458 (1.0); 7.443 (1.7); 7.439 (1.9); 7.419 (1.1); 7.324 (1.7); 7.304 (1.1); 7.289 (1.1); 7.284 (1.5); 7.270 (0.8); 7.266 (0.8); 7.200 (1.8); 7.188 (3.7); 7.182 (4.4); 7.163 (5.1); 7.143 (2.4); 7.053 (1.6); 7.029 (2.7); 7.009 (2.1); 6.906 (2.4); 6.885 (2.1); 4.222 (2.3); 4.206 (4.8); 4.190 (2.4); 3.334 (122.2); 3.129 (2.1); 3.113 (4.1); 3.097 (2.0); 2.890 (9.5); 2.730 (8.4); 2.671 (0.4); 2.666 (0.3); 2.506 (51.5); 2.502 (67.8); 2.497 (51.4); 2.328 (0.4); 2.003 (16.0); 0.000 (3.1)

### Example 63

¹H-NMR (400.0 MHz, d₆-DMSO): δ 10.170 (3.7); 8.821 (2.1); 8.809 (2.1); 8.206 (1.9); 8.187 (2.0); 7.952 (1.5); 7.738 (1.4); 7.726 (1.4); 7.718 (1.4); 7.706 (1.3); 7.400 (2.3); 7.386 (3.0); 7.379 (3.4); 7.365 (2.8); 7.328 (1.5); 7.193 (3.3); 7.183 (1.3); 7.160 (4.5); 7.143 (2.9); 7.137 (5.9); 7.115 (2.8); 7.057 (1.6); 7.028 (2.7); 7.009 (2.1); 6.903 (2.4); 6.882 (2.1); 4.201 (2.3); 4.185 (4.9); 4.169 (2.4); 3.343 (149.8); 3.078 (2.3); 3.062 (4.6); 3.046 (2.2); 2.890 (9.5); 2.731 (8.6); 2.506 (35.7); 2.502 (46.7); 2.498 (35.7); 2.023 (16.0); 0.000 (0.9)

### Example 64

¹H-NMR (400.0 MHz, d₆-DMSO): δ 10.171 (1.8); 8.854 (0.6); 8.844 (0.6); 8.822 (1.3); 8.810 (1.3); 8.205 (1.2); 8.185 (1.4); 7.952 (2.7); 7.736 (1.0); 7.723 (1.0); 7.705 (0.8); 7.505 (0.9); 7.500 (1.0); 7.487 (1.1); 7.482 (1.1); 7.468 (1.1); 7.464 (1.1); 7.449 (1.6); 7.445 (1.6); 7.436 (0.7); 7.337 (0.6); 7.323 (1.9); 7.319 (1.5); 7.303 (1.9); 7.298 (1.7); 7.284 (1.3); 7.279 (1.3); 7.265 (0.6); 7.260 (0.6); 7.218 (0.6); 7.189 (2.2); 7.167 (1.3); 7.146 (0.9); 7.053 (0.9); 7.031 (1.4); 7.012 (1.4); 6.994 (0.4); 6.923 (1.1); 6.903 (1.0); 4.822 (0.4); 4.818 (0.4); 4.805 (0.4); 4.788 (0.4); 4.775 (0.3); 4.635 (0.4); 4.246 (1.1); 4.230 (2.4); 4.214 (1.3); 3.350 (505.8); 3.231 (1.4); 3.215 (2.6); 3.199 (1.5); 2.890 (16.0); 2.731 (14.6); 2.672 (0.7); 2.507 (83.6); 2.503 (104.5); 2.499 (81.9); 2.330 (0.7); 2.058 (2.9); 2.018 (7.4); 1.984 (0.9); 1.936 (0.4); 1.904 (0.4); 1.320 (3.0); 1.305 (2.9); 1.258 (0.9); 1.241 (1.6); 1.224 (2.3); 1.211 (2.4); 1.179 (3.1); 1.164 (2.9); 1.121 (0.7); 1.085 (0.9); 1.021 (0.7); 0.956 (0.5); 0.940 (0.5); 0.000 (1.8)

### Example 65

¹H-NMR (400.0 MHz, d₆-DMSO): δ 10.169 (3.5); 8.823 (2.0); 8.820 (2.1); 8.811 (2.2); 8.808 (2.1); 8.209 (1.8); 8.189 (2.0); 7.952 (2.0); 7.737 (1.4); 7.725 (1.5); 7.717 (1.4); 7.705 (1.3); 7.449 (3.5); 7.406 (0.4); 7.369 (0.8); 7.351 (2.9); 7.332 (5.4); 7.326 (4.7); 7.312 (1.0); 7.305 (2.6); 7.300 (3.6); 7.295 (1.9); 7.287 (1.0); 7.282 (1.7); 7.277 (1.1); 7.193 (3.6); 7.167 (2.5); 7.146 (1.6); 7.057 (1.7); 7.035 (2.6); 7.015 (2.0); 6.910 (2.3); 6.890 (2.0); 4.223 (2.2); 4.207 (4.8); 4.191 (2.3); 3.332 (96.0); 3.099 (2.2); 3.084 (4.6); 3.068 (2.3); 3.051 (0.4); 2.890 (14.9); 2.730 (12.7); 2.671 (0.4); 2.524 (1.0); 2.510 (24.9); 2.506 (51.2); 2.502 (68.0); 2.497 (50.2); 2.493 (24.8); 2.328 (0.4); 2.324 (0.3); 2.027 (16.0); 1.977 (0.8); 1.232 (1.0); 1.216 (1.0); 0.000 (3.6)

### Example 67

¹H-NMR (400.0 MHz, d₆-DMSO): δ 10.144 (3.7); 8.824 (1.9); 8.821 (2.0); 8.813 (2.0); 8.809 (2.0); 8.203 (1.8); 8.184 (1.9); 7.952 (1.8); 7.739 (1.3); 7.727 (1.4); 7.720 (1.3); 7.708 (1.2); 7.361 (1.8); 7.355 (1.0); 7.339 (12.1); 7.331 (10.9); 7.315 (0.8); 7.309 (1.5); 7.292 (0.5); 7.195 (3.2); 7.165 (1.0); 7.145 (2.5); 7.125 (1.6); 7.060 (1.6); 7.012 (2.6); 6.992 (2.0); 6.901 (2.3); 6.881 (1.9); 4.706 (0.7); 4.691 (1.3); 4.676 (1.3); 4.660 (0.7); 3.335 (136.4); 3.005 (0.5); 2.988 (0.4); 2.971 (2.0); 2.954 (3.9); 2.940 (2.2); 2.920 (0.5); 2.906 (0.5); 2.890 (13.3); 2.730 (11.5); 2.671 (0.4); 2.524 (1.0); 2.510 (22.3); 2.506 (45.7); 2.502 (60.9); 2.497 (45.1); 2.493 (22.5); 2.328 (0.4); 2.029 (16.0); 1.839 (1.1); 1.251 (8.2); 1.236 (8.2); 1.183 (0.6); 1.168 (0.6); 0.000 (1.6)

### Example 68

¹H-NMR (400.0 MHz, d₆-DMSO): δ 10.148 (3.7); 8.821 (2.0); 8.812 (2.0); 8.809 (2.0); 8.205 (1.8); 8.186 (1.9); 7.952 (1.5); 7.738 (1.3); 7.726 (1.4); 7.719 (1.4); 7.707 (1.2); 7.388 (3.6); 7.344 (1.1); 7.329 (2.7); 7.322 (1.9); 7.306 (3.8); 7.291 (0.6); 7.281 (5.3); 7.277 (5.0); 7.265 (2.0); 7.259 (2.5); 7.244 (0.4); 7.241 (0.4); 7.194 (3.2); 7.169 (1.0); 7.149 (2.4); 7.128 (1.6); 7.058 (1.6); 7.015 (2.6); 6.996 (2.0); 6.915 (2.3); 6.894 (2.0); 4.751 (0.6); 4.733 (0.7); 4.718 (1.4); 4.703 (1.4); 4.688 (0.7); 3.376 (0.4); 3.340 (178.0); 3.309 (0.6); 3.021 (0.3); 3.004 (0.3); 2.986 (2.2); 2.976 (2.6); 2.970 (2.8); 2.963 (2.5); 2.942 (0.4); 2.904 (0.4); 2.890 (10.6); 2.731 (9.4); 2.671 (0.4); 2.510 (24.0); 2.506 (46.4); 2.502 (60.4); 2.498 (44.6); 2.329 (0.4); 2.042 (16.0); 1.849 (1.9); 1.251 (8.3); 1.237 (8.2); 1.188 (1.1); 1.173 (1.0); 0.000 (0.8)

### Example 69

¹H-NMR (400.0 MHz, d₆-DMSO): δ 10.141 (3.6); 8.823 (1.9); 8.819 (2.1); 8.811 (2.0); 8.808 (2.0); 8.200 (1.9); 8.181 (1.9); 7.952 (2.1); 7.736 (1.4); 7.725 (1.4); 7.717 (1.4); 7.705 (1.2); 7.460 (1.6); 7.455 (1.8); 7.443 (3.3); 7.440 (3.6); 7.425 (2.7); 7.420 (2.7); 7.339 (0.4); 7.326 (1.6); 7.308 (0.7); 7.304 (0.9); 7.289 (2.3); 7.285 (2.3); 7.272 (4.0); 7.267 (3.8); 7.254 (2.3); 7.249 (2.0); 7.235 (0.8); 7.230 (0.7); 7.203 (0.4); 7.191 (3.2); 7.158 (1.0); 7.138 (2.4); 7.118 (1.6); 7.055 (1.6); 7.010 (2.7); 6.990 (2.2); 6.887 (2.3); 6.867 (2.0); 4.765 (0.6); 4.749 (1.3); 4.732 (1.2); 4.718 (0.7); 3.391 (0.3); 3.374 (0.6); 3.344 (226.7); 3.318 (0.8); 3.204 (1.0); 3.186 (1.0); 3.170 (1.6); 3.152 (1.6); 3.066 (1.6); 3.053 (1.7); 3.032 (1.0); 3.018 (1.0); 2.890 (15.0); 2.731 (12.8); 2.672 (0.4); 2.525 (1.0); 2.511 (23.1); 2.507 (47.0); 2.502 (62.3); 2.498 (46.0); 2.493 (22.9); 2.456 (0.8); 2.329 (0.4); 2.217 (0.3); 2.208 (0.7); 2.041 (16.0); 1.848 (1.2); 1.567 (0.3); 1.551 (0.4); 1.306 (8.3); 1.291 (8.3); 1.232 (0.8); 1.217 (0.7); 0.000 (0.8)

### Example 70

¹H-NMR (400.0 MHz, d₆-DMSO): δ 10.161 (3.6); 8.826 (1.9); 8.823 (2.1); 8.814 (2.1); 8.811 (2.1); 8.207 (1.7); 8.188 (1.9); 7.952 (0.9); 7.741 (1.4); 7.729 (1.4); 7.722 (1.4); 7.710 (1.3); 7.455 (0.3); 7.387 (2.2); 7.383 (3.2); 7.363 (5.6); 7.331 (4.5); 7.326 (1.3); 7.312 (5.4); 7.292 (2.8); 7.216 (1.1); 7.213 (1.9); 7.210 (1.2); 7.197 (4.7); 7.180 (0.7); 7.177 (1.1); 7.154 (1.1); 7.134 (2.4); 7.114 (1.7); 7.062 (1.8); 7.050 (2.8); 7.031 (1.8); 6.836 (2.2); 6.816 (2.0); 4.632 (0.7); 4.618 (1.5); 4.603 (1.5); 4.588 (0.8); 3.345 (207.2); 3.308 (5.4); 3.294 (5.4); 2.890 (6.6); 2.731 (5.7); 2.671 (0.4); 2.525 (0.9); 2.511 (22.5); 2.507 (46.4); 2.502 (61.8); 2.498 (45.5); 2.494 (22.5); 2.329 (0.4); 2.129 (0.4); 2.075 (1.0); 2.040 (16.0); 1.411 (0.6); 1.394 (0.9); 1.383 (8.8); 1.368 (8.7); 1.223 (0.3); 0.000 (0.6)

### Example 71

¹H-NMR (400.0 MHz, d₆-DMSO): δ 10.202 (2.8); 8.981 (0.7); 8.977 (0.8); 8.969 (0.7); 8.965 (0.7); 8.823 (1.7); 8.814 (1.6); 8.811 (1.6); 8.626 (0.6); 8.606 (0.6); 8.217 (1.5); 8.198 (1.5); 7.952 (2.3); 7.842 (0.5); 7.829 (0.5); 7.822 (0.5); 7.810 (0.5); 7.741 (1.1); 7.729 (1.2); 7.722 (1.1); 7.710 (1.0); 7.590 (0.3); 7.565 (2.1); 7.562 (2.4); 7.552 (2.3); 7.549 (2.4); 7.455 (0.8); 7.337 (1.2); 7.320 (0.5); 7.216 (2.6); 7.201 (3.9); 7.178 (1.4); 7.082 (2.3); 7.065 (2.6); 7.059 (3.5); 7.050 (3.3); 7.046 (2.6); 7.037 (2.6); 6.500 (1.0); 6.479 (0.9); 5.334 (8.1); 4.859 (0.3); 4.843 (0.6); 4.827 (0.6); 4.812 (0.4); 3.399 (0.3); 3.346 (254.6); 3.304 (0.5); 2.890 (16.0); 2.731 (14.0); 2.672 (0.4); 2.525 (0.9); 2.511 (23.9); 2.507 (49.2); 2.502 (65.8); 2.498 (49.5); 2.329 (0.4); 2.325 (0.3); 2.125 (0.4); 2.104 (13.4); 2.085 (0.9); 1.935 (4.3); 1.224 (2.7); 1.209 (2.5); 1.127 (0.8); 0.000 (0.4)

### Example 72

¹H-NMR (400.0 MHz, d₆-DMSO): δ 10.169 (3.4); 8.824 (2.0); 8.815 (2.0); 8.813 (2.0); 8.214 (1.7); 8.195 (1.9); 7.952 (1.4); 7.743 (1.3); 7.731 (1.4); 7.724 (1.3); 7.712 (1.2); 7.348 (2.6); 7.342 (2.2); 7.337 (3.2); 7.332 (2.9); 7.203 (3.1); 7.181 (1.0); 7.160 (2.4); 7.140 (1.6); 7.068 (1.5); 7.031 (2.6); 7.012 (2.0); 6.971 (1.2); 6.963 (4.2); 6.957 (3.2); 6.952 (8.4); 6.913 (2.3); 6.892 (2.0); 4.687 (0.7); 4.672 (1.4); 4.657 (1.4); 4.643 (0.7); 3.392 (0.4); 3.349 (219.8); 3.298 (0.4); 3.194 (3.5); 3.183 (2.7); 3.177 (2.7); 2.890 (9.7); 2.731 (8.3); 2.672 (0.3); 2.525 (0.7); 2.511 (20.0); 2.507 (40.5); 2.503 (53.6); 2.498 (39.9); 2.494 (20.3); 2.329 (0.4); 2.104 (16.0); 1.284 (8.3); 1.269 (8.4); 0.000 (0.6)

### Example 73

¹H-NMR (400.0 MHz, d₆-DMSO): δ 10.230 (3.7); 8.829 (2.1); 8.820 (2.0); 8.817 (2.0); 8.229 (1.8); 8.209 (1.9); 7.953 (0.9); 7.799 (3.5); 7.779 (5.7); 7.750 (1.4); 7.737 (1.5); 7.730 (1.6); 7.718 (5.9); 7.697 (3.0); 7.344 (1.4); 7.209 (3.5); 7.194 (2.5); 7.174 (1.6); 7.088 (2.7); 7.073 (2.3); 7.070 (2.3); 6.978 (2.4); 6.958 (2.0); 5.278 (7.2); 3.341 (171.2); 2.890 (6.0); 2.731 (5.3); 2.672 (0.4); 2.525 (0.9); 2.511 (21.6); 2.507 (43.9); 2.503 (58.5); 2.498 (44.1); 2.494 (22.8); 2.334 (0.4); 2.329 (0.4); 2.175 (16.0); 0.000 (1.5)

### Example 74

¹H-NMR (300.2 MHz, CDCl₃): δ 8.743 (0.3); 7.491 (0.5); 7.477 (0.4); 7.466 (0.4); 7.268 (0.6); 7.192 (0.4); 7.153 (0.3); 7.082 (0.5); 7.009 (0.8); 6.827 (0.4); 5.288 (1.1); 4.108 (0.5); 4.084 (0.5); 2.623 (0.5); 2.607 (0.3); 2.594 (0.4); 2.579 (0.3); 2.564 (0.5); 2.195 (2.1); 2.021 (2.2); 1.437 (0.6); 1.422 (0.4); 1.407 (0.5); 1.394 (0.4); 1.378 (0.5); 1.269 (0.7); 1.245 (1.4); 1.222 (0.7); 1.190 (0.4); 1.169 (0.4); 0.984 (16.0); 0.954 (0.4); 0.871 (1.6); 0.862 (1.5); 0.077 (1.9)

### Example 75

¹H-NMR (300.2 MHz, CDCl₃): δ 8.783 (1.3); 8.770 (1.3); 8.077 (1.1); 8.052 (1.2); 7.620 (1.1); 7.548 (0.9); 7.531 (1.0); 7.523 (1.0); 7.506 (0.8); 7.442 (1.2); 7.415 (1.5); 7.264 (5.9); 7.208 (0.8); 7.194 (2.0); 7.181 (1.5); 7.154 (0.8); 7.012 (3.6); 6.849 (0.4); 6.830 (1.8); 6.782 (1.7); 6.754 (1.5); 5.296 (11.0); 4.795 (0.9); 4.783 (1.4); 4.770 (1.0); 4.755 (0.4); 4.148 (1.1); 4.125 (3.4); 4.101 (3.5); 4.077 (1.2); 2.166 (1.0); 2.161 (0.9); 2.128 (13.3); 2.037 (16.0); 1.897 (2.5); 1.889 (3.0); 1.874 (4.3); 1.863 (3.1); 1.857 (2.9); 1.839 (1.3); 1.833 (1.5); 1.828 (1.6); 1.814 (1.7); 1.797 (1.4); 1.787 (1.2); 1.772 (1.3); 1.747 (0.5); 1.739 (0.4); 1.702 (0.3); 1.689 (0.4); 1.654 (2.7); 1.642 (1.8); 1.635 (1.9); 1.627 (1.3); 1.622 (1.4); 1.613 (1.1); 1.584 (0.4); 1.278 (4.4); 1.271 (0.4); 1.255 (9.2); 1.231 (4.6); 1.210 (3.4); 1.190 (3.3); 0.085 (0.5); 0.073 (13.7); 0.060 (0.6); 0.000 (2.8)

### Example 76

¹H-NMR (300.2 MHz, CDCl₃): δ 8.772 (1.5); 8.758 (1.6); 8.067 (1.2); 8.042 (1.3); 7.705 (1.4); 7.538 (0.9); 7.521 (1.1); 7.513 (1.1); 7.493 (1.9); 7.464 (1.6); 7.265 (3.6); 7.238 (0.9); 7.211 (1.6); 7.177 (2.3); 6.994 (4.0); 6.812 (2.0); 6.753 (2.0); 6.726 (1.8); 6.201 (2.1); 6.181 (4.5); 6.161 (2.2); 5.294 (9.1); 4.692 (7.0); 4.671 (6.9); 4.142 (1.0); 4.118 (3.1); 4.094 (3.1); 4.070 (1.1); 2.154 (16.0); 2.031 (14.0); 1.674 (1.9); 1.306 (0.5); 1.275 (4.9); 1.266 (3.0); 1.251 (8.3); 1.227 (3.8); 0.903 (1.0); 0.881 (3.0); 0.858 (1.2); 0.074 (7.7); 0.062 (0.4); 0.000 (2.0)

### Example 77

¹H-NMR (300.2 MHz, CDCl₃): δ 8.740 (1.7); 8.727 (1.8); 8.021 (1.5); 7.995 (1.6); 7.783 (1.8); 7.492 (1.1); 7.475 (1.3); 7.467 (1.2); 7.450 (1.1); 7.352 (1.6); 7.325 (2.1); 7.263 (2.7); 7.181 (3.4); 7.153 (1.9); 7.126 (1.0); 6.998 (4.9); 6.816 (2.5); 6.746 (2.2); 6.718 (2.0); 5.286 (1.9); 4.131 (0.6); 4.107 (1.8); 4.084 (1.9); 4.060 (0.7); 3.768 (5.1); 3.748 (5.6); 2.206 (0.5); 2.141 (16.0); 2.021 (8.6); 1.895 (2.0); 1.857 (3.0); 1.833 (1.5); 1.821 (1.6); 1.796 (2.4); 1.785 (2.2); 1.772 (1.8); 1.763 (2.5); 1.753 (2.8); 1.697 (1.1); 1.385 (0.3); 1.375 (0.5); 1.343 (1.1); 1.334 (1.6); 1.302 (1.8); 1.293 (2.6); 1.268 (4.7); 1.259 (3.7); 1.254 (3.7); 1.245 (5.8); 1.237 (1.1); 1.232 (1.1); 1.221 (3.5); 1.215 (1.1); 1.192 (0.6); 1.183 (0.9); 1.172 (0.6); 1.152 (1.0); 1.142 (1.1); 1.104 (2.0); 1.070 (1.5); 1.064 (1.6); 1.031 (0.5); 1.026 (0.5); 0.903 (0.6); 0.881 (1.9); 0.858 (0.7); 0.090 (0.4); 0.078 (12.4); 0.066 (0.6); 0.000 (1.3)

### Example 78

¹H-NMR (300.2 MHz, CDCl₃): δ 7.261 (1.2); 7.190 (0.4); 7.007 (0.7); 6.825 (0.4); 6.788 (0.4); 4.058 (0.5); 4.035 (1.0); 4.012 (0.5); 2.156 (2.8); 1.782 (0.7); 1.759 (1.4); 1.735 (0.7); 1.617 (0.4); 1.004 (16.0); 0.072 (2.8); 0.000 (0.7)

### Example 79

¹H-NMR (300.2 MHz, CDCl₃): δ 8.796 (0.8); 8.783 (0.9); 8.095 (0.7); 8.070 (0.8); 7.566 (1.1); 7.549 (0.8); 7.523 (0.6); 7.492 (0.8); 7.465 (0.9); 7.263 (6.8); 7.220 (0.5); 7.196 (1.8); 7.166 (0.5); 7.014 (2.2); 6.832 (1.1); 6.787 (1.0); 6.760 (0.9); 5.299 (4.9); 4.415 (0.4); 4.395 (0.7); 4.375 (0.8); 4.355 (0.4); 4.153 (1.1); 4.129 (3.4); 4.105 (3.5); 4.081 (1.2); 2.167 (5.4); 2.159 (8.8); 2.041 (16.0); 1.772 (0.4); 1.765 (0.3); 1.754 (0.4); 1.750 (0.4); 1.743 (0.4); 1.738 (0.5); 1.733 (0.4); 1.722 (0.4); 1.716 (0.4); 1.613 (0.6); 1.603 (2.5); 1.595 (0.6); 1.582 (0.8); 1.564 (0.6); 1.558 (0.4); 1.541 (0.6); 1.530 (0.4); 1.522 (0.5); 1.505 (0.6); 1.487 (0.6); 1.480 (0.6); 1.475 (0.5); 1.462 (0.6); 1.456 (0.6); 1.450 (0.7); 1.439 (0.5); 1.427 (0.6); 1.418 (0.5); 1.405 (0.4); 1.395 (0.4); 1.307 (7.5); 1.287 (8.0); 1.281 (6.3); 1.266 (4.3); 1.263 (4.3); 1.257 (11.6); 1.233 (4.6); 1.004 (0.6); 0.969 (3.6); 0.959 (0.6); 0.945 (7.0); 0.921 (2.5); 0.904 (1.5); 0.882 (4.7); 0.859 (1.7); 0.071 (0.7); 0.000 (3.8)

### Example 80

¹H-NMR (300.2 MHz, CDCl₃): δ 7.551 (0.3); 7.392 (0.3); 7.276 (0.9); 7.026 (0.6); 4.148 (1.2); 4.124 (3.7); 4.100 (3.7); 4.077 (1.4); 2.221 (1.9); 2.036 (16.0); 1.641 (0.4); 1.278 (4.9); 1.263 (1.8); 1.254 (9.1); 1.247 (0.5); 1.245 (0.5); 1.243 (0.5); 1.230 (4.3); 0.904 (0.4); 0.882 (1.2); 0.859 (0.4); 0.000 (0.5)

### Example 81

¹H-NMR (300.2 MHz, CDCl₃): δ 8.746 (2.7); 8.739 (2.7); 8.730 (2.8); 8.038 (1.5); 8.017 (2.4); 7.996 (1.7); 7.762 (1.5); 7.681 (1.4); 7.500 (2.5); 7.493 (2.7); 7.479 (5.3); 7.453 (3.6); 7.260 (5.2); 7.254 (0.5); 7.251 (0.5); 7.224 (2.3); 7.215 (2.7); 7.204 (4.8); 7.195 (3.2); 7.184 (2.6); 7.012 (4.9); 7.001 (4.5); 6.988 (0.5); 6.874 (2.1); 6.848 (2.0); 6.830 (2.5); 6.819 (2.3); 6.764 (0.3); 6.651 (0.4); 6.626 (0.5); 6.575 (0.3); 6.521 (0.6); 6.495 (0.4); 3.450 (0.6); 3.436 (1.0); 3.420 (0.8); 3.398 (1.0); 3.382 (0.7); 3.147 (1.3); 3.135 (2.4); 3.112 (2.4); 3.100 (1.4); 3.065 (0.3); 3.052 (0.5); 3.003 (0.4); 2.367 (1.9); 2.353 (1.2); 2.316 (2.1); 2.288 (2.4); 2.264 (2.2); 2.228 (15.2); 2.202 (0.7); 2.187 (0.6); 2.178 (0.6); 2.168 (0.5); 2.099 (16.0); 2.070 (0.5); 1.978 (3.3); 1.963 (0.5); 1.951 (0.5); 1.938 (0.5); 1.920 (0.7); 1.910 (1.0); 1.882 (0.6); 1.870 (0.7); 1.856 (0.6); 1.837 (0.3); 1.811 (0.3); 1.797 (0.3); 1.653 (1.3); 1.618 (3.7); 1.594 (3.0); 1.576 (1.7); 1.564 (1.2); 1.557 (1.1); 1.548 (1.2); 1.522 (1.3); 1.502 (1.9); 1.496 (2.2); 1.489 (2.3); 1.464 (2.3); 1.430 (0.6); 1.421 (0.4); 1.402 (0.5); 1.378 (0.9); 1.359 (0.9); 1.342 (0.8); 1.336 (0.8); 1.317 (0.9); 1.298 (0.9); 1.272 (2.0); 1.256 (2.9); 1.225 (1.9); 1.205 (2.7); 1.198 (2.6); 1.181 (2.4); 1.174 (2.3); 1.164 (1.1); 1.157 (1.0); 1.133 (0.6); 0.995 (1.0); 0.961 (1.8); 0.899 (1.7); 0.880 (0.6); 0.866 (0.9); 0.088 (1.0); 0.076 (29.6); 0.063 (1.4); 0.050 (0.4); 0.000 (2.6)

### Example 82

¹H-NMR (300.2 MHz, CDCl₃): δ 8.793 (1.8); 8.779 (1.9); 8.096 (1.6); 8.071 (1.8); 7.657 (1.7); 7.614 (1.9); 7.588 (2.1); 7.562 (1.4); 7.546 (1.5); 7.537 (1.4); 7.520 (1.2); 7.422 (1.8); 7.396 (2.4); 7.295 (1.5); 7.263 (13.6); 7.242 (1.1); 7.200 (2.3); 7.018 (4.6); 6.836 (2.4); 6.739 (0.4); 5.116 (1.3); 5.093 (2.7); 5.069 (1.4); 4.176 (0.9); 4.154 (2.0); 4.149 (1.5); 4.132 (1.4); 4.127 (2.7); 4.105 (1.4); 3.965 (1.4); 3.941 (3.0); 3.917 (1.9); 3.914 (2.5); 3.890 (1.2); 3.152 (0.4); 3.128 (0.4); 2.426 (0.5); 2.403 (1.4); 2.380 (1.7); 2.363 (1.6); 2.358 (1.0); 2.340 (1.6); 2.317 (0.8); 2.276 (0.4); 2.207 (16.0); 2.051 (1.2); 2.041 (1.0); 2.027 (3.3); 2.003 (4.9); 1.980 (4.0); 1.957 (1.4); 1.764 (0.5); 1.740 (1.0); 1.714 (1.0); 1.699 (1.1); 1.674 (1.0); 1.649 (0.5); 1.622 (4.1); 1.277 (0.7); 1.253 (1.7); 1.233 (0.5); 1.229 (0.7); 1.089 (0.5); 1.065 (1.0); 1.041 (0.5); 0.071 (0.4); 0.000 (6.1)

### Example 83

¹H-NMR (300.2 MHz, CDCl₃): δ 8.762 (1.7); 8.749 (1.7); 8.050 (1.4); 8.024 (1.6); 7.693 (1.6); 7.520 (1.0); 7.504 (1.2); 7.496 (1.2); 7.479 (1.0); 7.408 (1.5); 7.381 (1.9); 7.263 (2.1); 7.190 (3.0); 7.163 (1.8); 7.136 (1.0); 7.007 (4.2); 6.825 (2.1); 6.762 (2.0); 6.734 (1.8); 4.236 (1.2); 4.217 (1.7); 4.198 (1.2); 4.179 (0.4); 4.114 (0.6); 4.090 (0.6); 2.148 (16.0); 2.028 (2.8); 1.734 (1.0); 1.710 (3.0); 1.690 (3.4); 1.685 (4.0); 1.665 (3.6); 1.652 (1.8); 1.640 (1.8); 1.626 (1.2); 1.617 (0.8); 1.607 (1.5); 1.588 (1.1); 1.580 (0.5); 1.561 (0.6); 1.544 (0.5); 1.523 (0.3); 1.504 (0.5); 1.480 (0.9); 1.456 (1.1); 1.437 (1.3); 1.431 (1.0); 1.423 (0.9); 1.414 (1.1); 1.405 (1.1); 1.395 (0.9); 1.381 (0.9); 1.371 (0.6); 1.360 (0.7); 1.338 (0.5); 1.315 (0.4); 1.308 (0.3); 1.272 (1.8); 1.267 (1.7); 1.249 (1.8); 1.225 (0.8); 0.976 (5.4); 0.952 (12.2); 0.932 (13.5); 0.908 (5.3); 0.881 (1.9); 0.858 (0.8); 0.000 (1.4)

### Example 84

¹H-NMR (300.2 MHz, CDCl₃): δ 8.731 (0.4); 8.718 (0.4); 8.012 (0.3); 7.986 (0.4); 7.791 (0.4); 7.354 (0.4); 7.327 (0.5); 7.265 (0.4); 7.180 (0.7); 7.157 (0.4); 6.997 (1.2); 6.815 (0.6); 6.793 (0.5); 6.766 (0.5); 2.114 (4.2); 2.013 (0.7); 1.870 (0.4); 1.842 (0.4); 1.821 (0.6); 1.794 (0.5); 1.469 (0.5); 1.460 (0.5); 1.420 (0.4); 1.411 (0.4); 1.268 (2.9); 1.248 (2.8); 1.239 (0.7); 0.953 (16.0); 0.889 (0.6)

### Example 85

¹H-NMR (300.2 MHz, CDCl₃): δ 8.735 (1.7); 8.721 (1.8); 8.018 (1.4); 7.992 (1.6); 7.812 (1.8); 7.484 (1.1); 7.468 (1.3); 7.460 (1.3); 7.443 (1.1); 7.341 (1.5); 7.315 (2.0); 7.265 (1.3); 7.185 (2.1); 7.170 (1.1); 7.142 (1.8); 7.115 (1.0); 7.003 (4.0); 6.821 (2.0); 6.772 (2.2); 6.745 (1.9); 4.260 (0.4); 4.240 (1.2); 4.219 (1.8); 4.198 (1.3); 4.177 (0.4); 2.204 (0.7); 2.180 (2.0); 2.152 (1.7); 2.126 (16.0); 2.018 (0.4); 1.968 (1.4); 1.910 (0.4); 1.890 (0.7); 1.866 (0.9); 1.850 (1.1); 1.829 (1.1); 1.810 (0.6); 1.778 (0.8); 1.772 (0.7); 1.764 (0.8); 1.745 (1.0); 1.739 (1.0); 1.727 (0.8); 1.713 (0.7); 1.699 (0.8); 1.677 (0.9); 1.661 (1.2); 1.651 (1.7); 1.638 (2.2); 1.623 (2.4); 1.611 (3.0); 1.594 (2.5); 1.587 (2.8); 1.578 (2.4); 1.567 (1.9); 1.558 (1.7); 1.538 (1.0); 1.525 (0.8); 1.512 (0.6); 1.491 (0.6); 1.468 (0.8); 1.441 (0.9); 1.428 (0.8); 1.402 (0.7); 1.376 (0.6); 1.351 (0.8); 1.330 (1.1); 1.321 (1.1); 1.312 (1.3); 1.283 (10.8); 1.263 (10.5); 1.218 (0.8); 1.198 (0.6); 0.000 (0.6)

### Example 86

¹H-NMR (300.2 MHz, CDCl₃): δ 8.732 (1.7); 8.719 (1.8); 8.011 (1.4); 7.985 (1.6); 7.795 (1.7); 7.483 (1.0); 7.466 (1.2); 7.458 (1.2); 7.441 (1.0); 7.354 (1.5); 7.328 (1.9); 7.263 (1.4); 7.178 (2.7); 7.145 (1.9); 7.118 (1.0); 6.996 (4.7); 6.814 (2.4); 6.770 (2.1); 6.742 (1.9); 4.388 (0.8); 4.368 (1.6); 4.347 (1.7); 4.327 (0.9); 2.125 (16.0); 1.803 (1.2); 1.789 (0.5); 1.782 (0.5); 1.777 (0.6); 1.770 (0.7); 1.768 (0.7); 1.756 (0.8); 1.750 (0.7); 1.744 (0.8); 1.739 (0.7); 1.726 (0.7); 1.706 (0.5); 1.633 (0.7); 1.621 (0.6); 1.614 (0.8); 1.602 (0.9); 1.588 (0.7); 1.582 (0.7); 1.557 (0.7); 1.538 (0.5); 1.488 (0.4); 1.473 (0.5); 1.463 (0.4); 1.457 (0.5); 1.445 (1.0); 1.428 (0.9); 1.413 (1.3); 1.406 (1.2); 1.386 (2.4); 1.373 (2.5); 1.363 (3.2); 1.354 (3.0); 1.342 (2.6); 1.331 (2.2); 1.302 (12.1); 1.282 (11.9); 0.936 (4.3); 0.913 (9.0); 0.902 (2.1); 0.889 (3.4); 0.000 (1.0)

### Example 87

¹H-NMR (300.2 MHz, CDCl₃): δ 8.751 (1.5); 8.737 (1.6); 8.035 (1.3); 8.010 (1.4); 7.729 (1.7); 7.507 (0.9); 7.490 (1.1); 7.482 (1.1); 7.465 (0.9); 7.395 (1.4); 7.368 (1.8); 7.263 (1.5); 7.186 (2.6); 7.160 (1.6); 7.133 (0.9); 7.004 (3.7); 6.840 (0.3); 6.822 (1.9); 6.769 (1.9); 6.742 (1.7); 4.364 (0.7); 4.344 (1.4); 4.324 (1.4); 4.304 (0.8); 2.190 (0.8); 2.137 (13.5); 1.798 (0.4); 1.780 (0.6); 1.774 (0.6); 1.757 (0.9); 1.743 (0.7); 1.736 (1.1); 1.716 (0.8); 1.698 (0.6); 1.653 (0.5); 1.634 (0.9); 1.616 (1.1); 1.603 (0.9); 1.598 (1.3); 1.589 (0.9); 1.581 (1.7); 1.571 (0.9); 1.560 (1.7); 1.554 (1.2); 1.537 (1.6); 1.516 (0.8); 1.493 (0.3); 1.412 (0.4); 1.395 (0.4); 1.391 (0.4); 1.369 (0.8); 1.352 (0.9); 1.348 (0.9); 1.332 (1.2); 1.304 (9.9); 1.284 (10.1); 1.272 (1.6); 1.268 (1.6); 1.254 (1.4); 1.247 (1.5); 1.227 (1.1); 1.206 (0.4); 0.910 (16.0); 0.888 (15.1); 0.000 (0.9)

### Example 88

¹H-NMR (300.2 MHz, CDCl₃): δ 8.754 (1.6); 8.740 (1.7); 8.039 (1.3); 8.013 (1.5); 7.678 (1.6); 7.506 (1.0); 7.489 (1.2); 7.482 (1.1); 7.464 (1.0); 7.424 (1.5); 7.397 (1.8); 7.315 (1.2); 7.291 (3.4); 7.287 (2.9); 7.277 (1.5); 7.268 (5.7); 7.254 (6.1); 7.248 (7.0); 7.237 (3.1); 7.226 (3.2); 7.210 (2.4); 7.198 (0.8); 7.184 (3.2); 7.158 (1.8); 7.131 (1.0); 7.001 (3.9); 6.819 (2.0); 6.770 (2.0); 6.742 (1.8); 4.619 (0.8); 4.598 (1.6); 4.578 (1.7); 4.558 (0.9); 4.138 (0.5); 4.115 (1.6); 4.091 (1.6); 4.067 (0.6); 3.130 (1.0); 3.109 (1.1); 3.084 (1.6); 3.064 (1.5); 2.903 (2.0); 2.882 (2.0); 2.858 (1.4); 2.837 (1.3); 2.168 (0.4); 2.122 (16.0); 2.027 (7.1); 1.703 (2.4); 1.319 (10.6); 1.299 (10.7); 1.271 (3.3); 1.247 (4.2); 1.224 (2.0); 0.903 (0.6); 0.881 (1.6); 0.858 (0.7); 0.000 (1.4)

### Example 89

¹H-NMR (300.2 MHz, CDCl₃): δ 8.723 (1.2); 8.710 (1.4); 8.002 (1.0); 7.977 (1.1); 7.812 (0.9); 7.460 (0.8); 7.444 (1.0); 7.435 (1.0); 7.419 (0.8); 7.370 (1.1); 7.350 (7.6); 7.328 (4.8); 7.304 (2.1); 7.278 (2.0); 7.270 (1.6); 7.259 (1.2); 7.254 (2.1); 7.248 (1.4); 7.235 (0.7); 7.228 (0.7); 7.220 (0.7); 7.209 (0.5); 7.178 (1.6); 7.030 (0.9); 6.996 (3.6); 6.976 (0.7); 6.814 (1.5); 6.623 (1.5); 6.595 (1.4); 5.338 (0.5); 5.316 (1.5); 5.295 (1.5); 5.274 (0.5); 4.128 (1.2); 4.105 (3.6); 4.081 (3.6); 4.057 (1.3); 2.230 (11.4); 2.093 (1.2); 2.086 (0.9); 2.016 (16.0); 1.795 (0.6); 1.676 (0.4); 1.651 (7.7); 1.630 (7.6); 1.609 (0.6); 1.587 (0.8); 1.563 (0.4); 1.264 (5.6); 1.241 (8.1); 1.217 (4.0); 0.902 (0.5); 0.881 (1.5); 0.857 (0.6); 0.000 (0.7)

### Example 90

¹H-NMR (400.1 MHz, d₆-DMSO): δ 10.191 (2.1); 8.831 (1.2); 8.827 (1.3); 8.819 (1.3); 8.816 (1.3); 8.235 (1.1); 8.215 (1.2); 7.749 (0.9); 7.737 (0.9); 7.729 (0.9); 7.717 (0.8); 7.520 (0.5); 7.361 (1.1); 7.350 (1.3); 7.345 (1.4); 7.267 (0.8); 7.251 (1.7); 7.224 (1.5); 7.215 (2.4); 7.205 (1.7); 7.186 (0.6); 7.079 (1.1); 4.480 (6.6); 3.706 (0.4); 3.691 (1.0); 3.675 (1.4); 3.660 (1.1); 3.645 (0.4); 3.320 (5.2); 2.509 (5.0); 2.504 (10.0); 2.500 (13.3); 2.495 (9.4); 2.491 (4.4); 2.201 (10.6); 1.171 (16.0); 1.156 (15.8); 0.000 (3.2)

### Example 91

¹H-NMR (400.1 MHz, CDCl₃): δ 8.817 (6.2); 8.813 (6.6); 8.805 (6.5); 8.801 (6.3); 8.042 (3.3); 8.011 (4.0); 7.991 (4.0); 7.690 (2.0); 7.675 (2.2); 7.558 (3.9); 7.546 (4.2); 7.538 (4.0); 7.526 (3.5); 7.518 (1.2); 7.505 (3.5); 7.484 (6.2); 7.464 (3.0); 7.309 (0.4); 7.259 (98.1); 7.210 (0.4); 7.170 (7.7); 7.033 (16.0); 6.995 (0.6); 6.906 (7.5); 6.897 (8.7); 6.885 (6.9); 5.297 (0.8); 4.891 (1.3); 4.880 (3.4); 4.873 (5.5); 4.864 (3.8); 4.853 (1.5); 1.917 (6.7); 1.905 (13.9); 1.898 (10.5); 1.891 (14.2); 1.879 (5.2); 1.861 (2.1); 1.856 (2.3); 1.849 (2.5); 1.839 (3.7); 1.830 (3.9); 1.819 (5.7); 1.807 (4.6); 1.802 (3.7); 1.790 (4.1); 1.770 (1.1); 1.762 (1.2); 1.742 (0.3); 1.684 (1.1); 1.674 (1.7); 1.671 (1.6); 1.659 (4.6); 1.649 (4.7); 1.642 (6.3); 1.633 (5.3); 1.624 (3.2); 1.618 (2.2); 1.604 (1.1); 1.598 (0.8); 1.592 (0.6); 1.536 (42.4); 1.256 (0.5); 0.070 (0.7); 0.008 (2.6); 0.000 (74.1); −0.009 (2.5); −0.049 (0.3)

### Example 92

¹H-NMR (300.2 MHz, CDCl₃): δ 8.826 (1.2); 8.821 (1.2); 8.810 (1.2); 8.805 (1.2); 8.065 (0.7); 8.023 (0.8); 7.997 (0.9); 7.681 (0.4); 7.657 (0.5); 7.574 (0.8); 7.557 (0.8); 7.548 (0.8); 7.531 (0.7); 7.511 (0.7); 7.484 (1.2); 7.456 (0.6); 7.262 (16.4); 7.225 (1.4); 7.043 (2.9); 6.884 (1.3); 6.861 (1.9); 6.856 (1.4); 5.302 (0.4); 4.294 (0.8); 4.275 (1.2); 4.256 (0.8); 1.768 (0.9); 1.743 (3.3); 1.724 (3.7); 1.718 (4.1); 1.699 (3.9); 1.694 (1.9); 1.674 (1.4); 1.560 (8.6); 0.982 (7.7); 0.958 (16.0); 0.933 (6.9); 0.011 (0.4); 0.000 (12.1); −0.011 (0.5)

### Example 93

¹H-NMR (300.2 MHz, CDCl₃): δ 8.825 (1.7); 8.820 (1.7); 8.809 (1.8); 8.804 (1.7); 8.067 (1.0); 8.022 (1.2); 7.996 (1.2); 7.678 (0.6); 7.653 (0.8); 7.573 (1.1); 7.557 (1.2); 7.547 (1.1); 7.531 (1.0); 7.511 (1.0); 7.483 (1.7); 7.455 (0.8); 7.262 (17.8); 7.225 (2.0); 7.043 (4.0); 6.886 (1.9); 6.860 (3.3); 4.359 (1.0); 4.339 (1.6); 4.320 (1.1); 4.301 (0.3); 1.762 (0.7); 1.737 (2.6); 1.718 (3.0); 1.712 (3.0); 1.704 (1.4); 1.693 (3.1); 1.670 (2.1); 1.655 (1.2); 1.650 (1.1); 1.644 (1.0); 1.636 (0.8); 1.625 (1.3); 1.606 (1.0); 1.598 (0.5); 1.578 (0.8); 1.567 (13.8); 1.494 (0.4); 1.489 (0.3); 1.470 (0.8); 1.451 (0.7); 1.445 (0.9); 1.438 (0.8); 1.432 (0.8); 1.426 (0.8); 1.421 (0.8); 1.413 (1.2); 1.408 (0.9); 1.398 (0.8); 1.389 (1.0); 1.377 (0.7); 1.364 (0.5); 1.354 (0.6); 1.331 (0.4); 0.979 (5.3); 0.955 (16.0); 0.931 (15.7); 0.907 (4.7); 0.011 (0.4); 0.000 (12.6); −0.011 (0.6)

### Example 94

¹H-NMR (300.2 MHz, CDCl₃): δ 8.823 (2.1); 8.818 (2.2); 8.807 (2.2); 8.802 (2.2); 8.062 (1.2); 8.019 (1.5); 7.994 (1.6); 7.694 (0.8); 7.669 (0.9); 7.570 (1.4); 7.554 (1.5); 7.544 (1.5); 7.527 (1.4); 7.518 (1.5); 7.490 (2.3); 7.462 (1.1); 7.262 (22.5); 7.222 (2.6); 7.040 (5.3); 6.910 (2.5); 6.881 (2.2); 6.858 (2.7); 4.471 (0.9); 4.451 (1.8); 4.431 (1.8); 4.411 (1.0); 1.828 (0.5); 1.807 (0.9); 1.782 (1.8); 1.761 (1.6); 1.757 (1.7); 1.736 (1.6); 1.728 (1.4); 1.709 (1.6); 1.703 (1.5); 1.684 (1.6); 1.661 (0.8); 1.638 (0.6); 1.561 (17.4); 1.325 (16.0); 1.305 (16.0); 1.283 (0.4); 1.259 (0.4); 1.015 (7.4); 0.990 (15.6); 0.965 (6.6); 0.011 (0.5); 0.000 (14.4); −0.011 (0.6)

### Example 95

¹H-NMR (300.2 MHz, CDCl₃): δ 8.617 (3.1); 8.612 (3.2); 8.047 (1.2); 7.788 (2.8); 7.684 (1.0); 7.658 (1.2); 7.512 (1.5); 7.485 (2.6); 7.457 (1.3); 7.262 (21.3); 7.168 (2.5); 6.986 (5.2); 6.904 (2.4); 6.875 (2.2); 6.803 (2.6); 4.469 (0.9); 4.449 (1.7); 4.429 (1.8); 4.409 (1.0); 2.472 (12.5); 1.828 (0.5); 1.807 (0.9); 1.804 (0.7); 1.782 (1.8); 1.761 (1.5); 1.757 (1.7); 1.736 (1.6); 1.727 (1.4); 1.708 (1.6); 1.702 (1.5); 1.684 (1.6); 1.660 (0.8); 1.656 (0.7); 1.637 (0.5); 1.564 (17.7); 1.325 (16.0); 1.305 (16.0); 1.015 (7.4); 0.990 (15.6); 0.965 (6.5); 0.011 (0.4); 0.000 (12.4); −0.011 (0.5)

### Example 96

¹H-NMR (300.2 MHz, CDCl₃): δ 8.035 (1.5); 7.984 (1.8); 7.956 (1.9); 7.623 (1.1); 7.580 (2.9); 7.553 (2.5); 7.513 (1.6); 7.486 (2.6); 7.458 (1.3); 7.261 (17.1); 7.121 (2.6); 6.940 (5.3); 6.916 (2.9); 6.887 (2.6); 6.759 (2.6); 4.469 (1.0); 4.449 (2.0); 4.429 (2.1); 4.409 (1.1); 1.826 (0.5); 1.805 (1.0); 1.780 (1.9); 1.759 (1.8); 1.755 (1.9); 1.734 (2.0); 1.727 (1.7); 1.708 (1.9); 1.702 (1.7); 1.683 (1.8); 1.659 (1.0); 1.637 (0.6); 1.547 (12.2); 1.324 (16.0); 1.303 (16.0); 1.012 (7.7); 0.988 (15.7); 0.963 (6.9); 0.011 (0.7); 0.000 (16.4); −0.011 (1.0)

### Example 97

¹H-NMR (300.2 MHz, CDCl₃): δ 8.032 (0.6); 7.982 (0.7); 7.956 (0.7); 7.664 (0.3); 7.643 (0.4); 7.580 (1.2); 7.553 (1.0); 7.519 (0.6); 7.491 (1.1); 7.463 (0.5); 7.261 (8.6); 7.121 (1.2); 6.940 (3.2); 6.908 (1.1); 6.759 (1.2); 4.671 (0.7); 4.651 (0.9); 4.631 (0.7); 1.546 (5.5); 1.378 (16.0); 1.358 (15.9); 0.011 (0.3); 0.000 (9.0); −0.011 (0.3)

### Example 98

¹H-NMR (300.2 MHz, CDCl₃): δ 8.618 (1.6); 8.613 (1.6); 8.042 (0.6); 7.787 (1.4); 7.703 (0.5); 7.676 (0.6); 7.517 (0.7); 7.490 (1.3); 7.462 (0.6); 7.261 (7.1); 7.168 (1.2); 6.985 (2.4); 6.924 (1.2); 6.895 (1.1); 6.803 (1.2); 5.300 (0.5); 4.671 (0.7); 4.651 (1.0); 4.631 (0.7); 2.472 (6.3); 1.556 (3.7); 1.379 (16.0); 1.358 (15.9); 1.335 (0.4); 0.000 (7.7); −0.011 (0.3)

### Example 99

¹H-NMR (300.2 MHz, CDCl₃): δ 8.824 (1.0); 8.819 (1.1); 8.808 (1.1); 8.803 (1.0); 8.055 (0.6); 8.019 (0.8); 7.994 (0.8); 7.713 (0.4); 7.688 (0.4); 7.569 (0.7); 7.553 (0.7); 7.544 (0.7); 7.525 (1.0); 7.495 (1.1); 7.467 (0.6); 7.261 (7.8); 7.221 (1.2); 7.039 (2.5); 6.930 (1.2); 6.901 (1.1); 6.857 (1.3); 4.673 (0.7); 4.653 (0.9); 4.632 (0.7); 1.553 (4.1); 1.379 (16.0); 1.359 (15.9); 0.000 (8.5); −0.011 (0.3)

### Example 100

¹H-NMR (300.2 MHz, CDCl₃): δ 8.824 (2.7); 8.812 (2.7); 8.808 (2.7); 8.087 (1.6); 8.030 (1.9); 8.005 (2.1); 7.779 (1.0); 7.751 (1.3); 7.577 (1.8); 7.561 (1.9); 7.551 (1.9); 7.538 (2.9); 7.511 (3.1); 7.483 (1.6); 7.448 (2.3); 7.422 (10.0); 7.397 (7.4); 7.371 (3.1); 7.361 (2.0); 7.355 (2.7); 7.347 (1.4); 7.332 (2.4); 7.320 (0.6); 7.316 (0.6); 7.310 (0.7); 7.260 (24.8); 7.216 (2.9); 7.034 (5.7); 6.978 (3.2); 6.950 (2.9); 6.852 (2.9); 5.299 (2.1); 5.193 (16.0); 1.548 (18.9); 1.253 (0.7); 0.011 (1.0); 0.000 (25.6); −0.011 (1.2)

### Example 101

¹H-NMR (300.2 MHz, CDCl₃): δ 8.823 (2.4); 8.819 (2.5); 8.808 (2.6); 8.803 (2.6); 8.066 (1.5); 8.020 (1.8); 7.995 (1.9); 7.661 (1.0); 7.634 (1.2); 7.606 (0.4); 7.571 (1.7); 7.555 (1.8); 7.546 (1.7); 7.529 (1.5); 7.369 (15.2); 7.354 (15.7); 7.342 (4.3); 7.328 (1.4); 7.323 (1.7); 7.309 (2.5); 7.295 (2.4); 7.288 (1.2); 7.280 (2.4); 7.270 (1.1); 7.260 (24.3); 7.207 (3.0); 7.025 (6.1); 6.843 (3.0); 6.743 (3.0); 6.715 (2.7); 5.424 (0.9); 5.403 (2.9); 5.382 (3.0); 5.360 (1.0); 1.672 (16.0); 1.651 (16.0); 1.549 (15.8); 0.011 (0.8); 0.000 (24.3); −0.011 (1.1)

### Example 102

¹H-NMR (300.2 MHz, CDCl₃): δ 8.615 (3.9); 8.051 (1.6); 7.790 (3.4); 7.652 (1.3); 7.624 (1.5); 7.368 (15.3); 7.354 (15.4); 7.338 (4.3); 7.329 (1.6); 7.322 (1.7); 7.309 (3.5); 7.294 (2.5); 7.288 (1.4); 7.280 (2.5); 7.260 (29.8); 7.156 (2.8); 6.974 (5.8); 6.791 (3.0); 6.736 (3.0); 6.708 (2.8); 5.423 (0.9); 5.402 (2.9); 5.381 (3.0); 5.360 (1.0); 5.299 (0.7); 2.475 (15.0); 2.382 (0.3); 1.672 (15.8); 1.651 (16.0); 1.629 (0.6); 1.551 (21.0); 0.011 (1.0); 0.000 (30.7); −0.011 (1.4)

### Example 103

¹H-NMR (300.2 MHz, CDCl₃): δ 8.039 (1.3); 7.984 (1.4); 7.956 (1.5); 7.606 (0.9); 7.581 (3.0); 7.554 (2.1); 7.366 (12.7); 7.351 (16.0); 7.338 (3.3); 7.326 (1.2); 7.321 (1.7); 7.308 (2.8); 7.295 (2.4); 7.280 (2.1); 7.274 (1.0); 7.260 (18.7); 7.108 (2.3); 6.927 (4.8); 6.746 (4.2); 6.721 (2.3); 5.422 (0.7); 5.400 (2.4); 5.379 (2.4); 5.358 (0.8); 5.299 (2.6); 1.671 (12.7); 1.650 (12.8); 1.544 (10.9); 0.011 (0.6); 0.000 (19.0); −0.011 (0.9)

### Example 104

¹H-NMR (300.2 MHz, d₆-DMSO): δ 10.186 (3.2); 8.831 (1.9); 8.826 (2.1); 8.815 (2.1); 8.810 (2.1); 8.226 (1.7); 8.200 (1.9); 7.749 (1.4); 7.733 (1.4); 7.723 (1.4); 7.707 (1.3); 7.398 (1.6); 7.217 (3.6); 7.138 (1.0); 7.112 (2.6); 7.085 (1.9); 7.037 (1.8); 6.994 (2.7); 6.969 (1.8); 6.880 (2.3); 6.856 (1.9); 3.337 (29.5); 3.096 (2.5); 3.076 (7.1); 3.056 (3.3); 2.513 (1.6); 2.507 (3.6); 2.501 (5.2); 2.495 (3.9); 2.489 (2.0); 2.149 (16.0); 1.902 (2.6); 1.881 (7.5); 1.871 (4.2); 1.861 (3.1); 1.355 (1.5); 1.234 (0.4); 0.000 (4.1)

### Example 105

¹H-NMR (300.2 MHz, d₆-DMSO): δ 10.431 (9.2); 8.852 (9.4); 8.838 (9.6); 8.287 (8.0); 8.261 (9.0); 7.876 (9.6); 7.849 (11.6); 7.830 (9.0); 7.803 (10.8); 7.782 (6.6); 7.766 (6.8); 7.758 (6.5); 7.741 (5.6); 7.558 (6.8); 7.531 (11.2); 7.505 (5.0); 7.413 (7.0); 7.233 (14.9); 7.053 (7.4); 4.064 (0.4); 4.040 (1.2); 4.017 (1.2); 3.992 (0.4); 3.922 (1.3); 3.896 (4.1); 3.873 (6.6); 3.845 (4.3); 3.823 (1.8); 3.333 (38.5); 2.797 (0.5); 2.584 (72.6); 2.502 (32.9); 2.368 (0.5); 1.990 (5.1); 1.901 (2.4); 1.879 (5.5); 1.856 (11.8); 1.835 (16.0); 1.814 (12.8); 1.788 (7.3); 1.747 (3.7); 1.717 (5.9); 1.691 (8.5); 1.669 (8.4); 1.637 (7.1); 1.609 (9.8); 1.587 (8.8); 1.561 (4.8); 1.198 (1.2); 1.174 (2.6); 1.150 (1.4); 0.000 (23.2)

### Example 106

¹H-NMR (300.2 MHz, d₆-DMSO): δ 10.123 (3.1); 8.845 (1.8); 8.840 (1.9); 8.829 (2.0); 8.824 (1.8); 8.232 (1.6); 8.206 (1.8); 7.764 (1.3); 7.748 (1.4); 7.738 (1.3); 7.722 (1.2); 7.406 (1.4); 7.226 (3.2); 7.045 (2.5); 7.020 (2.3); 6.994 (1.3); 6.612 (2.3); 6.587 (2.0); 6.518 (2.0); 6.491 (1.8); 4.357 (1.6); 4.328 (1.7); 3.354 (21.1); 3.300 (0.5); 3.280 (0.9); 3.253 (0.8); 3.233 (0.5); 2.534 (2.1); 2.528 (4.4); 2.522 (6.1); 2.516 (4.5); 2.510 (2.2); 2.215 (0.4); 2.017 (13.8); 1.607 (0.9); 1.582 (3.4); 1.558 (4.7); 1.536 (3.7); 1.512 (1.3); 0.941 (7.5); 0.916 (16.0); 0.891 (6.6)

### Example 107

¹H-NMR (300.2 MHz, d₆-DMSO): δ 10.133 (3.3); 8.843 (1.8); 8.838 (2.0); 8.827 (2.0); 8.822 (1.9); 8.231 (1.6); 8.206 (1.8); 7.761 (1.3); 7.745 (1.4); 7.735 (1.3); 7.719 (1.2); 7.402 (1.5); 7.221 (3.3); 7.075 (1.1); 7.048 (2.6); 7.041 (2.1); 7.022 (1.4); 6.662 (2.3); 6.636 (2.1); 6.560 (2.1); 6.533 (2.0); 4.605 (1.8); 4.584 (1.9); 4.060 (0.3); 4.037 (0.3); 3.814 (0.5); 3.794 (0.9); 3.773 (0.9); 3.754 (0.5); 3.353 (17.6); 2.533 (2.9); 2.528 (6.1); 2.522 (8.2); 2.515 (6.0); 2.510 (2.9); 2.002 (16.0); 1.948 (0.8); 1.722 (1.8); 1.705 (1.3); 1.675 (0.6); 1.636 (0.6); 1.603 (1.3); 1.563 (3.2); 1.552 (2.5); 1.534 (1.5); 1.218 (0.4); 1.194 (0.9); 1.171 (0.4)

### Example 108

¹H-NMR (300.2 MHz, d₆-DMSO): δ 10.180 (1.2); 8.853 (0.7); 8.849 (0.8); 8.838 (0.8); 8.833 (0.8); 8.243 (0.7); 8.217 (0.8); 7.773 (0.5); 7.757 (0.6); 7.747 (0.5); 7.731 (0.5); 7.416 (0.6); 7.236 (1.4); 7.225 (0.4); 7.198 (1.0); 7.172 (0.9); 7.136 (1.1); 7.113 (0.6); 7.055 (0.7); 7.017 (0.9); 7.013 (0.9); 6.991 (0.7); 3.355 (5.1); 2.656 (16.0); 2.534 (0.6); 2.528 (1.3); 2.522 (1.8); 2.516 (1.4); 2.510 (0.7); 2.221 (6.7)

### Example 109

¹H-NMR (300.2 MHz, d₆-DMSO): δ 10.132 (2.9); 8.851 (1.5); 8.847 (1.6); 8.835 (1.7); 8.831 (1.6); 8.236 (1.4); 8.210 (1.6); 7.771 (1.1); 7.755 (1.2); 7.745 (1.1); 7.729 (1.0); 7.416 (1.3); 7.235 (2.9); 7.204 (1.6); 7.189 (2.3); 7.181 (5.1); 7.164 (0.9); 7.141 (0.3); 7.122 (1.7); 7.112 (1.4); 7.100 (1.0); 7.091 (0.9); 7.055 (1.4); 5.779 (4.4); 3.484 (0.7); 3.462 (1.1); 3.439 (0.7); 3.344 (25.6); 2.540 (15.4); 2.528 (7.1); 2.522 (9.0); 2.516 (6.6); 2.510 (3.3); 2.230 (16.0); 2.010 (0.4); 1.738 (1.2); 1.718 (1.2); 1.705 (1.2); 1.689 (0.9); 1.680 (1.0); 1.665 (1.0); 1.639 (1.5); 1.619 (1.3); 1.580 (1.0); 1.550 (1.1); 1.540 (1.1); 1.528 (1.3); 1.502 (1.7); 1.489 (1.5); 1.469 (1.3); 1.446 (1.1); 1.423 (0.7)

### Example 110

¹H-NMR (300.2 MHz, d₆-DMSO): δ 10.354 (2.6); 9.026 (3.2); 9.022 (3.2); 8.890 (3.3); 8.884 (3.4); 8.846 (1.9); 8.842 (2.1); 8.831 (2.1); 8.826 (2.1); 8.255 (1.7); 8.223 (3.0); 8.216 (3.6); 7.771 (1.3); 7.755 (1.4); 7.745 (1.4); 7.729 (1.2); 7.606 (1.7); 7.581 (2.3); 7.426 (1.3); 7.411 (2.2); 7.400 (2.7); 7.374 (1.5); 7.288 (2.5); 7.266 (1.8); 7.231 (4.3); 7.050 (2.0); 5.760 (1.3); 3.328 (19.7); 2.514 (3.0); 2.508 (6.4); 2.502 (8.8); 2.496 (6.5); 2.490 (3.2); 2.163 (16.0); 0.000 (5.8)

### Example 111

¹H-NMR (300.2 MHz, d₆-DMSO): δ 10.312 (3.1); 8.844 (1.7); 8.839 (1.8); 8.828 (1.9); 8.823 (1.8); 8.254 (1.5); 8.229 (1.7); 7.767 (1.2); 7.751 (1.3); 7.741 (1.2); 7.725 (1.1); 7.469 (1.1); 7.458 (1.4); 7.448 (1.3); 7.438 (1.7); 7.424 (0.4); 7.411 (1.6); 7.325 (0.5); 7.299 (2.7); 7.289 (3.2); 7.278 (5.9); 7.264 (0.8); 7.230 (3.6); 7.197 (2.8); 7.194 (3.9); 7.190 (2.8); 7.050 (1.8); 6.982 (4.2); 6.978 (4.1); 3.326 (11.4); 2.513 (2.2); 2.507 (4.7); 2.501 (6.6); 2.495 (4.8); 2.489 (2.4); 2.288 (16.0); 2.267 (14.6); 2.264 (14.3); 0.000 (9.1); −0.011 (0.4)

### Example 112

¹H-NMR (300.2 MHz, d₆-DMSO): δ 10.300 (2.0); 8.842 (1.2); 8.837 (1.2); 8.826 (1.3); 8.822 (1.2); 8.254 (1.1); 8.228 (1.2); 7.762 (0.8); 7.746 (0.9); 7.736 (0.8); 7.721 (0.7); 7.541 (1.1); 7.517 (1.3); 7.411 (1.0); 7.346 (0.8); 7.321 (1.6); 7.295 (0.9); 7.231 (2.2); 7.206 (1.5); 7.184 (1.1); 7.051 (1.1); 3.331 (69.0); 2.645 (13.1); 2.514 (3.1); 2.508 (6.7); 2.502 (9.3); 2.496 (6.9); 2.490 (3.4); 2.108 (16.0); 1.989 (0.5); 0.000 (5.5)

### Example 113

¹H-NMR (300.2 MHz, CDCl₃): δ 8.813 (1.7); 8.800 (1.8); 8.138 (1.5); 8.113 (1.6); 8.040 (1.1); 7.773 (1.5); 7.749 (1.6); 7.615 (1.7); 7.607 (1.7); 7.585 (7.3); 7.583 (7.5); 7.551 (1.2); 7.490 (7.1); 7.315 (0.8); 7.290 (2.1); 7.278 (0.8); 7.275 (0.9); 7.274 (1.0); 7.272 (1.2); 7.262 (58.0); 7.254 (3.3); 7.252 (3.6); 7.251 (3.8); 7.248 (3.4); 7.246 (3.5); 7.224 (1.4); 7.201 (1.9); 7.019 (3.7); 6.837 (1.8); 5.301 (2.6); 4.600 (0.6); 4.577 (1.5); 4.555 (2.1); 4.533 (1.7); 4.510 (0.7); 4.133 (0.3); 4.109 (0.3); 2.330 (16.0); 2.102 (0.3); 2.046 (1.6); 1.642 (0.4); 1.580 (38.0); 1.558 (37.7); 1.515 (0.6); 1.497 (0.4); 1.283 (0.7); 1.259 (1.7); 1.254 (1.7); 1.236 (0.6); 0.011 (1.3); 0.000 (42.5); −0.008 (1.5); −0.009 (1.3); −0.011 (1.8); −0.021 (0.4)

### Example 114

¹H-NMR (300.2 MHz, d₆-DMSO): δ 10.300 (1.8); 8.845 (1.0); 8.840 (1.0); 8.829 (1.1); 8.824 (1.0); 8.262 (0.9); 8.236 (1.0); 7.768 (0.7); 7.752 (0.7); 7.742 (0.7); 7.726 (0.7); 7.614 (5.8); 7.468 (0.9); 7.447 (1.5); 7.442 (1.5); 7.427 (1.5); 7.417 (1.1); 7.331 (1.0); 7.306 (1.3); 7.280 (0.5); 7.236 (2.0); 7.056 (1.0); 3.387 (0.4); 3.364 (1.0); 3.342 (1.6); 3.327 (22.0); 3.319 (2.0); 3.304 (0.3); 3.296 (0.5); 2.513 (2.1); 2.507 (4.6); 2.501 (6.4); 2.495 (4.6); 2.489 (2.2); 2.291 (7.6); 1.989 (0.6); 1.391 (16.0); 1.368 (15.7); 1.174 (0.4); 0.000 (5.4)

### Example 115

¹H-NMR (300.2 MHz, CDCl₃): δ 8.787 (1.4); 8.774 (1.5); 8.088 (1.2); 8.063 (1.3); 7.610 (1.4); 7.557 (0.9); 7.541 (1.1); 7.533 (1.0); 7.516 (0.8); 7.425 (1.3); 7.398 (6.0); 7.391 (5.7); 7.382 (2.9); 7.364 (0.6); 7.354 (3.5); 7.260 (6.2); 7.222 (2.6); 7.215 (2.5); 7.194 (2.0); 7.187 (2.0); 7.181 (2.4); 7.085 (0.8); 7.058 (1.5); 7.031 (0.8); 6.999 (4.5); 6.817 (2.2); 6.460 (2.0); 6.433 (1.9); 5.671 (0.6); 5.650 (2.0); 5.629 (2.0); 5.608 (0.7); 2.270 (16.0); 2.249 (0.7); 2.142 (0.8); 1.997 (4.5); 1.635 (11.5); 1.614 (11.6); 1.598 (1.1); 1.576 (0.8); 0.000 (3.9)

### Example 116

¹H-NMR (300.2 MHz, CDCl₃): δ 8.808 (1.7); 8.795 (1.8); 8.114 (1.5); 8.090 (1.7); 7.619 (1.7); 7.606 (0.8); 7.594 (2.0); 7.578 (1.4); 7.562 (1.4); 7.552 (1.5); 7.536 (1.6); 7.517 (1.5); 7.261 (48.0); 7.223 (2.2); 7.213 (2.8); 7.197 (1.4); 7.137 (2.4); 7.112 (1.5); 7.030 (4.7); 6.848 (2.3); 2.888 (1.1); 2.878 (1.2); 2.847 (1.3); 2.837 (1.2); 2.297 (16.0); 2.045 (0.4); 1.918 (0.7); 1.905 (1.0); 1.871 (2.1); 1.832 (1.5); 1.614 (1.2); 1.590 (2.0); 1.581 (2.4); 1.570 (2.4); 1.550 (34.0); 1.528 (2.6); 1.523 (2.6); 1.441 (1.1); 1.426 (0.9); 1.412 (1.1); 1.398 (1.2); 1.383 (1.5); 1.369 (1.3); 1.341 (1.0); 1.326 (0.7); 1.314 (0.6); 1.283 (0.6); 1.266 (1.4); 1.259 (1.4); 0.897 (13.3); 0.882 (2.8); 0.858 (0.9); 0.737 (12.1); 0.707 (0.7); 0.011 (1.1); 0.000 (37.7); −0.011 (1.8)

### Example 117

¹H-NMR (300.2 MHz, CDCl₃): δ 8.800 (0.6); 8.787 (0.6); 8.101 (0.5); 8.075 (0.6); 7.632 (0.6); 7.605 (0.7); 7.572 (0.5); 7.546 (0.8); 7.533 (0.7); 7.262 (6.2); 7.195 (0.9); 7.182 (0.4); 7.154 (0.7); 7.127 (0.4); 7.012 (1.7); 6.952 (0.9); 6.925 (0.7); 6.830 (0.8); 2.190 (6.2); 2.133 (0.6); 2.110 (0.8); 2.088 (0.6); 1.586 (3.9); 1.258 (16.0); 1.059 (11.6); 1.036 (11.2); 0.000 (3.6)

### Example 118

¹H-NMR (300.2 MHz, CDCl₃): δ 8.805 (1.5); 8.792 (1.6); 8.112 (1.3); 8.087 (1.4); 7.607 (0.3); 7.578 (1.2); 7.552 (2.0); 7.537 (2.1); 7.526 (2.2); 7.498 (1.9); 7.262 (30.6); 7.243 (1.1); 7.214 (1.8); 7.196 (2.1); 7.014 (3.5); 6.832 (1.8); 6.783 (1.9); 6.756 (1.8); 4.089 (1.5); 4.066 (4.6); 4.043 (4.8); 4.020 (1.7); 2.219 (0.3); 2.182 (16.0); 2.008 (3.2); 1.570 (11.3); 1.460 (7.6); 1.437 (15.6); 1.413 (7.5); 0.011 (0.7); 0.000 (17.8); −0.011 (0.7)

### Example 119

¹H-NMR (300.2 MHz, CDCl₃): δ 8.804 (1.5); 8.790 (1.6); 8.114 (1.1); 8.089 (1.3); 7.672 (1.3); 7.645 (1.5); 7.608 (1.3); 7.586 (1.3); 7.568 (1.2); 7.544 (0.9); 7.281 (0.8); 7.262 (13.9); 7.254 (1.8); 7.227 (0.9); 7.169 (1.8); 6.987 (3.6); 6.805 (1.8); 6.759 (2.1); 6.732 (1.9); 4.417 (1.8); 4.390 (5.6); 4.363 (5.7); 4.336 (2.0); 2.215 (16.0); 2.006 (1.9); 1.581 (9.1); 0.000 (7.4); −0.011 (0.3)

### Example 120

¹H-NMR (300.2 MHz, CDCl₃): δ 8.805 (0.4); 8.792 (0.4); 8.115 (0.3); 8.090 (0.4); 7.583 (0.9); 7.558 (0.8); 7.263 (8.0); 7.240 (0.5); 7.183 (0.5); 7.001 (1.0); 6.819 (0.5); 6.772 (0.6); 6.744 (0.5); 4.247 (0.9); 4.226 (1.9); 4.205 (1.0); 4.156 (1.1); 4.132 (3.4); 4.108 (3.5); 4.085 (1.2); 2.699 (0.5); 2.678 (0.9); 2.664 (0.5); 2.657 (0.5); 2.643 (1.0); 2.622 (0.5); 2.608 (0.3); 2.172 (4.4); 2.045 (16.0); 2.034 (0.3); 1.574 (2.6); 1.307 (0.7); 1.283 (5.5); 1.259 (12.1); 1.236 (4.6); 0.904 (1.7); 0.882 (5.7); 0.859 (2.0); 0.000 (5.6)

### Example 121

¹H-NMR (400.1 MHz, d₆-DMSO): δ 10.163 (3.0); 8.828 (1.8); 8.824 (1.9); 8.816 (1.9); 8.813 (1.8); 8.225 (1.7); 8.205 (1.8); 7.745 (1.3); 7.733 (1.3); 7.726 (1.3); 7.714 (1.1); 7.349 (1.3); 7.234 (0.8); 7.225 (1.1); 7.213 (4.4); 7.196 (7.4); 7.189 (2.2); 7.180 (1.5); 7.078 (1.4); 3.310 (2.0); 3.250 (0.7); 3.247 (0.8); 3.228 (1.3); 3.208 (0.8); 3.205 (0.8); 2.503 (10.2); 2.498 (13.4); 2.494 (9.7); 2.227 (16.0); 2.017 (1.1); 2.008 (1.3); 2.005 (1.3); 1.991 (1.4); 1.964 (0.7); 1.808 (0.9); 1.798 (1.4); 1.788 (1.8); 1.778 (1.3); 1.771 (1.2); 1.756 (0.7); 1.746 (0.5); 1.718 (0.5); 1.712 (0.5); 1.698 (0.8); 1.689 (1.2); 1.679 (1.6); 1.672 (1.7); 1.660 (1.7); 1.653 (1.0); 1.575 (0.6); 1.554 (1.1); 1.547 (1.1); 1.540 (1.1); 1.533 (1.3); 1.525 (1.2); 1.512 (0.9); 1.504 (0.9); 1.484 (0.4)

### Example 122

¹H-NMR (300.2 MHz, d₆-DMSO): δ 10.195 (1.0); 8.835 (0.6); 8.830 (0.7); 8.819 (0.7); 8.814 (0.7); 8.237 (0.6); 8.211 (0.6); 7.757 (0.5); 7.741 (0.5); 7.731 (0.5); 7.715 (0.4); 7.397 (0.6); 7.255 (0.6); 7.231 (0.9); 7.217 (1.4); 7.162 (0.6); 7.156 (0.4); 7.137 (0.9); 7.111 (0.4); 7.036 (0.6); 6.951 (0.7); 6.926 (0.6); 3.922 (0.9); 3.328 (16.0); 2.513 (0.6); 2.507 (1.3); 2.500 (1.8); 2.494 (1.3); 2.488 (0.6); 2.319 (5.6); 2.256 (1.0); 1.951 (0.4); 0.967 (0.4); 0.953 (1.0); 0.946 (1.0); 0.939 (0.5); 0.933 (0.6); 0.925 (1.0); 0.918 (1.0); 0.905 (0.4); 0.632 (0.4); 0.619 (1.0); 0.613 (1.2); 0.601 (1.1); 0.595 (1.0); 0.581 (0.3); 0.000 (1.0)

### Example 123

¹H-NMR (300.2 MHz, d₆-DMSO): δ 10.234 (2.2); 8.983 (1.9); 8.966 (2.0); 7.928 (1.5); 7.911 (1.5); 7.392 (1.1); 7.298 (1.3); 7.212 (2.3); 7.199 (0.7); 7.172 (1.8); 7.145 (1.6); 7.119 (3.4); 7.111 (2.2); 7.088 (1.0); 7.032 (1.1); 6.941 (1.5); 6.882 (1.5); 6.858 (1.3); 4.864 (0.5); 4.846 (1.0); 4.827 (0.6); 3.322 (16.0); 2.513 (3.3); 2.507 (7.2); 2.501 (9.9); 2.495 (7.2); 2.489 (3.4); 2.053 (11.9); 1.989 (1.0); 1.932 (0.4); 1.910 (0.9); 1.894 (1.0); 1.888 (0.9); 1.858 (0.8); 1.839 (0.4); 1.762 (1.2); 1.749 (1.6); 1.737 (2.1); 1.721 (2.0); 1.701 (1.3); 1.674 (0.6); 1.656 (0.5); 1.633 (0.8); 1.622 (0.7); 1.602 (1.2); 1.582 (0.6); 1.174 (0.5); 0.011 (0.4); 0.000 (13.5); −0.011 (0.5)

### Example: In Vivo Preventive Test on Puccinia recondita (Brown Rust on Wheat)

Solvent: 5% by volume of Dimethyl sulfoxide
10% by volume of Acetone

Emulsifier: 1 μl of Tween® 80 per mg of active ingredient

The active ingredients are made soluble and homogenized in a mixture of Dimethyl sulfoxide/Acetone/Tween® 80 and then diluted in water to the desired concentration.

The young plants of wheat are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Puccinia recondita spores. The contaminated wheat plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test, the following compounds according to the invention showed efficacy of at least 70% at a concentration of 100 ppm of active ingredient: 2; 3; 5; 6; 7; 9; 10; 11; 12; 13; 14; 15; 16; 17; 19; 20; 21; 22; 23; 24; 25; 26; 27; 28; 29; 30; 31; 32; 33; 34; 35; 36; 37; 38; 39; 40; 41; 42; 44; 45; 46; 47; 48; 49; 50; 51; 52; 53; 54; 55; 56; 57; 58; 60; 61; 62; 63; 65; 67; 68; 69; 70; 72; 73; 74; 75; 76; 77; 78; 79; 80; 81; 82; 83; 84; 85; 86; 87; 88; 89; 91; 92; 93; 94; 95; 96; 98; 99; 100; 101; 110; 111; 112; 113; 114; 115; 116; 117; 118; 119; 120; 121; 122

### Example: In Vivo Preventive Test on Uromyces appendiculatus (Bean Rust)

Solvent: 5% by volume of Dimethyl sulfoxide
10% by volume of Acetone

Emulsifier: 1 μl of Tween® 80 per mg of active ingredient

The active ingredients are made soluble and homogenized in a mixture of Dimethyl sulfoxide/Acetone/Tween® 80 and then diluted in water to the desired concentration.

The young plants of bean are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Uromyces appendiculatus spores. The contaminated bean plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test, the following compounds according to the invention showed efficacy of at least 70% at a concentration of 100 ppm of active ingredient: 2; 3; 4; 5; 6; 8; 9; 10; 11; 12; 13; 14; 15; 16; 17; 20; 21; 22; 23; 24; 25; 26; 27; 28; 29; 30; 31; 32; 33; 34; 35; 38; 39; 44; 46; 47; 48; 49; 50; 51; 52; 53; 56; 57; 58; 60; 61; 62; 63; 65; 67; 68; 69; 70; 72; 73; 74; 75; 76; 78; 79; 80; 81; 83; 84; 85; 86; 87; 88; 89; 91; 92; 93; 94; 99; 106; 109; 110; 111; 114; 115; 116; 117; 118; 119; 120; 121; 122

### Example: In Vivo Preventive Test on Phakopsora Test (Soybeans)

Solvent: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous spore suspension of the causal agent of soybean rust (Phakopsora pachyrhizi) and stay for 24 h without light in an incubation cabinet at approximately 24° C. and a relative atmospheric humidity of 95%.

The plants remain in the incubation cabinet at approximately 24° C. and a relative atmospheric humidity of approximately 80% and a day/night interval of 12 h.

The test is evaluated 7 days after the inoculation. 0% means an efficacy which corresponds to that of the untreated control, while an efficacy of 100% means that no disease is observed.

In this test, the following compounds according to the invention showed efficacy of at least 70% at a concentration of 10 ppm of active ingredient: 12; 14; 15; 25; 30; 47; 49; 51; 52; 60; 63; 67; 68; 69; 72; 79; 83; 84; 85; 86; 87; 88; 89; 92; 94; 116; 121

### Experimental Examples

Process (a)

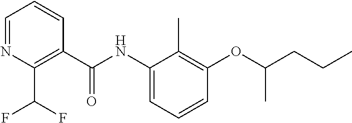 [SMILES: CCCC(C)Oc1cccc(NC(=O)c2cccnc2C(F)F)c1C]

2-(difluoromethyl)-N-[2-methyl-3-(pentan-2-yloxy)phenyl]nicotinamide (ex 79)

In a microwave sealable tube, propanephosphonic anhydride (50% in AcOEt, 1.83 ml, 3.09 mmol, 3 eq) is added to a solution of 2-(difluoromethyl)-5-methylnicotinic acid (214 mg, 1.24 mmol, 1.2 eq) and 2-methyl-3-(pentan-2-yloxy)aniline (200 mg, 1.035 mmol, 1 eq.) in 10 ml of THF. The tube is sealed and the reaction is microwaved 10 min at 150° C. The resulting solution is quenched with aq. sat. K₂CO₃, extracted with AcOEt, washed with aq. Sat. NH₄Cl and filtered through alumina. The solvent is evaporated to give pure material (75%)

Process (c)

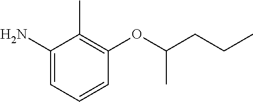

2-methyl-3-[(4-methylpentan-2-yl)oxy]aniline

To a solution of 3-amino-o-cresol (2 g, 16.24 mmol, 1 eq.) in THF (50 ml) were added sequentially triphenylphosphine (4.26 g, 16.24 mmol, 1 eq.), triethylamine (1.654 g, 2.26 ml, 16.24 mmol, 1 eq.) and 4-methylpentan-2-ol (1.99 g, 19.48 mmol, 1.2 eq.). Then DIAD (3.28 g, 16.24 mmol, 1 eq.) was added dropwise and upon complete addition the mixture was stirred further 16 h. The reaction was quenched with 1N HCl and extracted with AcOEt. The organic layers were dried over MgSO₄ and evaporated under reduced pressure. The crude was purified by chromatography on silica gel to give pure material (54%).

Process (d)

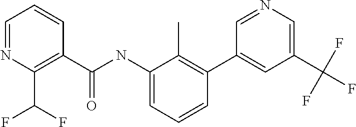 [SMILES: Cc1c(NC(=O)c2cccnc2C(F)F)cccc1-c1cncc(C(F)(F)F)c1]

2-(difluoromethyl)-N-{2-methyl-3-[5-(trifluoromethyl)pyridin-3-yl]phenyl}nicotinamide (ex 110)

In a microwave sealable tube are introduced N-(3-bromo-2-methylphenyl)-2-(difluoromethyl)nicotinamide (90 mg, 0.264 mmol, 1 eq.), [1,1′-bis(diphenylphosphino) ferrocene]dichloropalladium(II) (19 mg, 0.026 mmol, 0.1 eq.) and [5-(trifluoromethyl)pyridin-3-yl]boronic acid (75 mg, 0.39 mmol, 1.5 eq). The tube is purged with argon then dioxane (1.5 ml) and a 2M aqueous solution of sodium carbonate (0.8 ml) are added. After sealing, the reaction is microwaved at 100° C. for 10 min. The resulting solution is quenched with water, extracted with AcOEt and dried avec MgSO₄ before evaporation to dryness. The crude is purified by preparative HPLC to give pure material (44%).

1. 2-Difluoromethyl-nicotin(thio)carboxanilides derivative of formula (I) 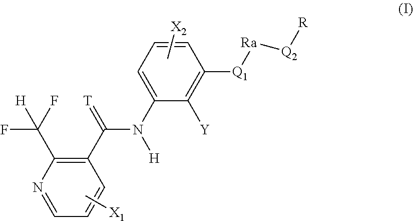
wherein
X₁ represents hydrogen, halogen, CN, NO₂, C₁-C₃-alkyl, C₁-C₃-haloalkyl, C₁-C₃-alkyloxy, C₁-C₃-haloalkyloxy, tri(C₁-C₃)alkylsilyl, C₁-C₃-alkylsulfanyl, C₁-C₃-haloalkylsulfanyl, C₁-C₃-alkylsulfonyl; C₁-C₃-haloalkylsulfonyl, C₁-C₃-alkylamino; di-C₁-C₃-alkylamino;
X₂ represents H, halogen, CN, NO₂, C₁-C₆-alkyl, C₁-C₆-haloalkyl, C₃-C₈-cycloalkyl, C₃-C₈-halocycloalkyl, C₁-C₆-alkyloxy, C₁-C₆-haloalkyloxy, tri(C₁-C₈)alkylsilyl, C₁-C₆-alkylsulfanyl, C₁-C₆-haloalkyl sulfanyl, C₁-C₆-alkylsulfonyl; C₁-C₆-haloalkylsulfonyl, C₁-C₈-alkylamino; di-C₁-C₈-alkylamino;
Y represents hydrogen, halogen, C₁-C₃-alkyl, C₁-C₃-haloalkyl,
Q₁ represents O, S(O)ₘ, NR¹ or a bond;
m represents 0, 1 or 2;
R¹ represents hydrogen, C₁-C₆-alkyl, C₃-C₈-cycloalkyl, C₁-C₈-halogenoalkyl;
Ra represents a group; 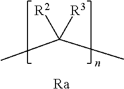
R² and R³ which can be the same or different independently of one another, represent hydrogen, halogen, CN, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy, C₁-C₈-alkylsulfanyl, C₃-C₈-cycloalkylsulfanyl, C₁-C₈-halogenoalkylsulfanyl, C₃-C₈-halogenocycloalkylsulfanyl;
R² and R³ also together with the carbon atom to which they are linked can form a C₃-C₇-cycloalkyl or a carbonyl;
n represents 0, 1, 2, 3, 4 or 5;
Q₂ represents O, S(O)ₘ, NR¹ or a bond
R represents C₁-C₈-alkyl, C₃-C₈-cycloalkyl, C₁-C₈-halogenoalkyl, C₃-C₈-halogenocycloalkyl, C₂-C₈-alkenyl, C₃-C₈-cycloalkenyl, C₂-C₈-halogenoalkenyl, C₃-C₈-halogenocycloalkenyl, C₂-C₈-alkynyl, C₃-C₈-cycloalkynyl, C₂-C₈-halogenoalkynyl, C₃-C₈-halogenocycloalkynyl, a phenyl which can be substituted by up to 5 groups X₃, a saturated or unsaturated 5, 6 or 7 membered heterocycle which can be substituted by up to 5 groups X₃, a group ═N—O—R⁴, C₇-C₁₂-bicycloalkyl
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl or tri(C₁-C₈)alkylsilyl-C₁-C₈-alkyl, or two vicinal substituents X₃ may be —OCH₂O—, —OCF₂O—, —O(CH₂)₂O—, —O(CF₂)₂O— or —N═CH—S—,
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃, C₁-C₈-alkyl phenyl which can be substituted by up to 5 groups X₃
T is O, S;
And/or a salt, solvate, N-oxide, solvate of a salt and/or N-oxide thereof.

2. 2-Difluoromethyl-nicotin(thio) carboxanilide derivative of formula (I) and/or a salt, solvate, N-oxide, solvate of a salt and/or N-oxide thereof according to claim 1, wherein
X₁ represents hydrogen, halogen, CN, NO₂, C₁-C₃-alkyl, C₁-C₃-haloalkyl
X₂ represents H, halogen, CN, NO₂, C₁-C₆-alkyl, C₁-C₆-haloalkyl, C₁-C₆-alkyloxy, C₁-C₆-haloalkyloxy,
Y represents hydrogen, halogen, methyl, ethyl, propyl, difluoromethyl, trifluoromethyl,
Q₁ represents O, S, NH or a bond,
Ra represents a group 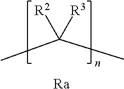
R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl, C₁-C₈-alkoxy, C₃-C₈-cycloalkoxy, C₁-C₈-halogenoalkyloxy, C₃-C₈-halogenocycloalkyloxy;
n represents 0, 1, 2, 3, 4 or 5;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃, a group ═N—O—R⁴;
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl;
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃, C₁-C₈-alkyl phenyl which can be substituted by up to 5 groups X₃;
T represents O;
Q₂ represents O, S or a bond.

3. 2-Difluoromethyl-nicotin(thio) carboxanilide derivative of formula (I) and/or a salt, solvate, N-oxide, solvate of a salt and/or N-oxide thereof according to claim 1, wherein
X₁ represents hydrogen, Chlorine, fluorine, bromine, methyl, trifluoromethyl;
X₂ represents hydrogen, Chlorine, fluorine, bromine, methyl, trifluoromethyl;
Y represents chlorine, methyl, trifluoromethyl,
Q₁ represents O, S, NH or a bond,
Ra represents a group 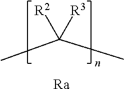
R² and R³ which can be the same or different independently of one another, represent hydrogen, halogen, C₁-C₈-alkyl, C₃-C₈-cycloalkyl, C₁-C₈-halogenoalkyl, C₃-C₈-halogenocycloalkyl;
n represents 0, 1, 2 or 3;
R represents C₁-C₈-alkyl, C₃-C₈-cycloalkyl, C₁-C₈-halogenoalkyl, C₃-C₈-halogenocycloalkyl, tri(C₁-C₈)alkylsilyl, C₂-C₈-alkenyl, C₂-C₈-halogenoalkenyl, C₂-C₈-alkynyl, C₂-C₈-halogenoalkynyl, a phenyl which can be substituted by up to 5 groups X₃, a saturated or unsaturated 5, 6 or 7 membered heterocycle which can be substituted by up to 5 groups X₃, a group ═N—O—R⁴,
X₃ which can be the same or different, independently represents halogen, CN, NO₂, C₁-C₈-alkyl, C₁-C₈-alkoxy, C₁-C₈-alkylsulfanyl, C₁-C₈-halogenoalkyl, C₁-C₈-halogenoalkoxy, tri(C₁-C₈)alkylsilyl,
R⁴ represents hydrogen, C₁-C₈-alkyl, C₃-C₈-cycloalkyl, C₁-C₈-halogenoalkyl, C₃-C₈-halogenocycloalkyl, a phenyl which can be substituted by up to 5 groups X₃, C₁-C₈-alkyl phenyl which can be substituted by up to 5 groups X₃,
T represents O,
Q₂ represents O, S or a bond.

4. Composition for controlling phytopathogenic harmful fungi, comprising a content of at least one compound of the formula (I) and/or a salt, solvate, N-oxide, solvate of a salt and/or N-oxide thereof according to claim 1, in addition to one or more extenders and/or surfactants.

5. Method for controlling phytopathogenic harmful fungi, comprising applying one or more compounds of the formula (I) and/or a salt, solvate, N-oxide, solvate of a salt and/or N-oxide thereof according to claim 1 to the phytopathogenic harmful fungi and/or a habitat thereof.

6. A product comprising one or more compounds of formula (I) and/or one or more salts, solvates, N-oxides, solvates of salts and/or N-oxides thereof according to claim 1 for control of phytopathogenic harmful fungi.

7. Process for producing a composition for controlling phytopathogenic harmful fungi, comprising mixing one or more compounds of the formula (I) and/or one or more salts, solvates, N-oxides, solvates of salts and/or N-oxides thereof according to claim 1 with one or more extenders and/or surfactants.

8. A product comprising one or more compounds of the formula (I) and/or one or more salts, solvates, N-oxides, solvates of salts and/or N-oxides thereof according to claim 1 for treatment of transgenic plants, seed and/or of seed of transgenic plants.
